(19) United States
(12) Patent Application Publication
(10) Pub. No.: US 2026/0002109 A1
(43) Pub. Date: Jan. 1, 2026

(54) SYSTEMS AND PROCESSES FOR SKIN TISSUE GROWTH AND ANALYSIS

(71) Applicant: Outer Biosciences, Inc., Malden, MA (US)

(72) Inventors: Chris HINOJOSA, Malden, MA (US); Kyung-Jin Jang, Andover, MA (US); Stanley King, Boston, MA (US)

(21) Appl. No.: 19/320,607

(22) Filed: Sep. 5, 2025

Related U.S. Application Data
(63) Continuation of application No. PCT/US24/18684, filed on Mar. 6, 2024.
(60) Provisional application No. 63/488,673, filed on Mar. 6, 2023.

(51) Int. Cl. C12M 3/00; C12M 1/00; C12M 1/12; C12N 5/071

(52) U.S. Cl. CPC C12M 23/42; C12M 25/14; C12M 29/04; C12M 37/04; C12N 5/0698

(57) ABSTRACT

This disclosure relates to skin tissue systems, devices, and methods. Systems described include ones comprising: a tissue sample; an insert; and a porous layer configured to promote mass transport into the tissue sample; wherein the skin tissue system is capable of supporting tissue viability for at least one week. Methods described include ones for skin tissue analysis comprising: contacting a skin tissue array with at least one test agent; measuring a plurality of datasets following the contacting; mapping the plurality of datasets based on a scoring panel result; and generating at least one threshold criteria for selecting the therapeutic agent from the at least one test agent based on the mapped datasets. Such innovations described herein provide for an ex vivo model tissue system and use of the model tissue system for analytical activities such as screening of nutrients, pharmaceutical activity, and toxicity in tissue samples.

### CROSS-REFERENCE

This application is a continuation of International Application No. PCT/US24/18684, filed Mar. 6, 2024 which claims priority to U.S. Provisional Application Ser. No. 63/488,673 filed on Mar. 6, 2023, which are incorporated herein by reference in their entirety for all purposes.

### BACKGROUND

Animal and human trials are expensive, timely, and come with an inherent degree of experimental risk to living organisms. In the area of compound or biologic testing, there is currently no effective testing system for skin research. Commercially available methods for skin tissue analysis are lacking data consistency, as the skin models used to attain such data are lacking in longevity that hampers their ability to provide clinically relevant endpoints. Thus, there is a need for a reliable analytical workflow using model systems to reduce the costs associated with live organism trials, while also affording meaningful tissue survival longevity to provide clinically relevant endpoints.

### BRIEF SUMMARY

This section includes a summary of the claims in the commonly accepted definition of a comprehensive and usually brief recapitulation of the claims. It should be appreciated that these embodiments are merely illustrative, that embodiments are not limited to operating in accordance with the specific examples shown in the figures and discussed below, and that other embodiments are possible. The following embodiments are put forth so as to provide those of ordinary skill in the art with a complete disclosure and description of how to make and use the ex vivo human skin models, compositions, and methods of use and manufacture as described in some aspects and embodiments herein, and are not intended to limit the scope of the various aspects and embodiments herein.

In some aspects, the techniques described herein relate to an ex vivo skin tissue system, including: an insert including at least one opening and a wall: a skin tissue sample sealed inside the insert: and a porous layer positioned inside the insert, wherein the porous layer has a first side facing the at least one opening and a second side facing opposite the at least one opening of the insert, wherein at least one pore of the porous layer includes a gyroid, and wherein the porous layer includes an arrangement relative to the skin tissue sample so as to promote mass transport of reagents and/or fluid into the skin tissue sample for supporting viability of the skin tissue sample for at least one week. In some aspects, the techniques described herein relate to an ex vivo skin tissue system, further including an adipose layer positioned inside the insert, wherein the porous layer is in contact with at least one of the adipose layer and the skin tissue sample. In some aspects, the techniques described herein relate to an ex vivo skin tissue system, wherein the adipose layer includes human tissue from a same source as the skin tissue sample. In some aspects, the techniques described herein relate to an ex vivo skin tissue system, wherein the adipose layer includes human adipose-derived stem cells, adipocytes, or pre-adipocytes. In some aspects, the techniques described herein relate to an ex vivo skin tissue system, wherein the gyroid includes a gyroid infill. In some aspects, the techniques described herein relate to an ex vivo skin tissue system, further including at least one of an inlet channel and outlet channel positioned lateral to the porous layer. In some aspects, the techniques described herein relate to an ex vivo skin tissue system, further including a gel layer in contact with the second side, wherein the gel layer is configured to receive one or more of adipose-derived cells, dermal cells, and endothelial cells. In some aspects, the techniques described herein relate to an ex vivo skin tissue system, further including a cell composition seeded within the porous layer between the first side and the second side. In some aspects, the techniques described herein relate to an ex vivo skin tissue system, wherein the seeded cell composition includes one or more of endothelial cells, primary cells, iPSCs, keratinocytes, fibroblasts, melanocytes, resident immune cells, circulating immune cells, stem cells, adipocytes, microbes, or a combination thereof. In some aspects, the techniques described herein relate to an ex vivo skin tissue system, wherein the skin tissue sample includes at least one of an epidermis layer, a dermis layer, and a hypodermis layer. In some aspects, the techniques described herein relate to an ex vivo skin tissue system, wherein the porous layer includes a porosity gradient of at least one gyroid, and wherein the porosity gradient includes a lower density of pores in a center of the porous layer relative to an outer boundary of the porous layer. In some aspects, the techniques described herein relate to an ex vivo skin tissue system, wherein an interior surface of the wall perpendicular to the first side of the porous layer includes a plurality of pores. In some aspects, the techniques described herein relate to an ex vivo skin tissue system, wherein the plurality of pores penetrate a desired thickness of the wall. In some aspects, the techniques described herein relate to an ex vivo skin tissue system, wherein the skin tissue sample is cultured, thereby forming an ex vivo skin model.

In some aspects, the techniques described herein relate to an ex vivo skin tissue system, including: an insert including at least one opening and a wall: a skin tissue sample and an adipose layer positioned inside the insert: an adhesive film configured to at least contact or seal the adipose layer within the insert: and a porous layer positioned inside the insert, wherein the porous layer has a first side facing the at least one opening and a second side facing opposite the at least one opening of the insert, and wherein the porous layer includes a structure and arrangement relative to the skin tissue sample so as to promote mass transport of reagents and/or fluid into the skin tissue sample for supporting viability of the skin tissue sample for at least one week. In some aspects, the techniques described herein relate to an ex vivo skin tissue system, wherein the adipose layer includes human tissue from a same source as the skin tissue sample. In some aspects, the techniques described herein relate to an ex vivo skin tissue system, wherein the adipose layer includes one or more of human adipose-derived stem cells. adipocytes, or pre-adipocytes. In some aspects, the techniques described herein relate to an ex vivo skin tissue system, wherein the porous layer includes a plurality of pores. In some aspects, the techniques described herein relate to an ex vivo skin tissue system, further including at least one of an inlet channel and outlet channel positioned lateral to the porous layer. In some aspects, the techniques described herein relate to an ex vivo skin tissue system, further including a gel layer in contact with the second side, wherein the gel layer is configured to receive one or more of adipose-derived cells, dermal cells, and endothelial cells. In some aspects, the techniques described herein relate to an ex vivo skin tissue system, further including a cell composition seeded within the porous layer between the first side and the second side. In some aspects, the techniques described herein relate to an ex vivo skin tissue system, wherein the seeded cell composition includes one or more of endothelial cells, primary cells, iPSCs, keratinocytes, fibroblasts, melanocytes, resident immune cells, circulating immune cells, stem cells, adipocytes, microbes, or a combination thereof. In some aspects, the techniques described herein relate to an ex vivo skin tissue system, wherein the skin tissue sample includes at least one of an epidermis layer, a dermis layer, and a hypodermis layer. In some aspects, the techniques described herein relate to an ex vivo skin tissue system, wherein the porous layer includes a porosity gradient, and wherein the porosity gradient includes a lower density of pores in a center of the porous layer relative to an outer boundary of the porous layer. In some aspects, the techniques described herein relate to an ex vivo skin tissue system, wherein an interior surface of the wall perpendicular to the first side of the porous layer includes a plurality of pores. In some aspects, the techniques described herein relate to an ex vivo skin tissue system, wherein the plurality of pores penetrate a desired thickness of the wall. In some aspects, the techniques described herein relate to an ex vivo skin tissue system, wherein the skin tissue sample is cultured, thereby forming an ex vivo skin model.

In some aspects, provided herein is an ex vivo skin tissue system, comprising: an insert including at least one opening and a wall; a skin tissue sample sealed inside the insert; and a porous layer positioned inside the insert, wherein the porous layer has a first side facing the at least one opening and a second side facing opposite the at least one opening of the insert, and wherein the porous layer includes a structure and arrangement relative to the skin tissue sample so as to promote mass transport of reagents and/or fluid into the skin tissue sample for supporting viability of the skin tissue sample for at least one week. In some aspects, the ex vivo skin tissue system further comprises an adipose layer positioned inside the insert. In some aspects, the porous layer is in contact with at least one of the adipose layer and the tissue sample. In some aspects, the ex vivo skin tissue system further comprises at least one of an inlet channel and outlet channel positioned lateral to the porous layer. In some aspects, the ex vivo skin tissue system further comprises a gel layer in contact with the second side, wherein the gel layer is configured to receive one or more of adipose-derived cells, dermal cells, and endothelial cells. In some aspects, the gel layer includes a gel material. In some aspects, the gel layer includes extracellular matrix. In some aspects, the ex vivo skin tissue system further comprises an adhesive film configured for at least one of contacting and sealing the adipose layer. In some aspects, the ex vivo skin tissue system further comprises a cell composition seeded within the porous layer between the first side and the second side. In some aspects, the ex vivo skin tissue system further comprises a gel layer in contact with the second side. In some aspects, the seeded cells include one or more of endothelial cells, primary cells, iPSCs, keratinocytes, fibroblasts, melanocytes, resident immune cells, circulating immune cells, stem cells, adipocytes, and microbes. In some aspects, the insert includes a first threading along an interior surface of the wall perpendicular to the first side of the porous layer. In some aspects, the tissue sample is sealed using an adhesive, wherein the adhesive is selected from a medical grade super glue and a UV curable glue. In some aspects, the tissue sample includes at least one of an epidermis layer, a dermis layer, and a hypodermis layer. In some aspects, the porous layer includes a porosity gradient. In some aspects, the porous layer includes a plurality of pores, wherein each of the plurality of pores is a gyroid. In some aspects, the ex vivo skin tissue system further comprises at least one standoff along the second side of the porous layer. In some aspects, an interior surface of the wall perpendicular to the first side of the porous layer includes a plurality of pores. In some aspects, each of the plurality of pores is a gyroid. In some aspects, the plurality of pores penetrate a desired thickness of the wall. In some aspects, the ex vivo skin tissue system further comprises growth media in contact with the tissue sample. In some aspects, the growth media is maintained at between 32-37 degrees Celsius. In some aspects, the growth media includes at least one test agent. In some aspects, the ex vivo skin tissue system further comprises at least one test agent contacted to the tissue sample, wherein the contacting is one or more of topical application, subcutaneous application, systemic application, intradermal administration, infusion, perfusion, and injection.

In some aspects, provided herein is an ex vivo skin tissue system, comprising: an insert including at least one opening(s) and a wall; a sealing unit along an inner wall of the insert; a tissue sample positioned inside the insert via the sealing unit; and a porous layer positioned inside the insert, wherein the porous layer is configured to promote mass transport of reagents and/or fluid into the tissue sample, and wherein the porous layer has a first side facing the at least one opening(s) and a second side facing opposite the at least one opening(s) of the insert; and wherein the ex vivo skin tissue system is capable of supporting tissue viability for longer than a week. In some aspects, the ex vivo skin tissue system further comprises an adipose layer positioned inside the insert. In some aspects, the ex vivo skin tissue system further comprises a cell composition seeded within the porous layer between the first side and the second side. In some aspects, the ex vivo skin tissue system further comprises a gel layer in contact with the second side. In some aspects, the seeded cells include one or more of endothelial cells, primary cells, iPSCs, keratinocytes, fibroblasts, melanocytes, resident immune cells, circulating immune cells, stem cells, adipocytes, and microbes. In some aspects, the insert includes a first threading along an interior surface of the wall perpendicular to the first side of the porous layer. In some aspects, the sealing unit includes a compression unit, the compression unit including a second threading along an outer perimeter configured for mating the first threading. In some aspects, the compression unit provides either (1) complete sealing or (2) partial sealing of the tissue sample. In some aspects, the compression unit includes an injection port in fluid communication with a microchannel. In some aspects, the sealing unit is an adhesive, wherein the adhesive is selected from a medical grade super glue and a UV curable glue. In some aspects, the porous layer is in contact with at least one of the adipose layer and the tissue sample. In some aspects, the ex vivo skin tissue system further comprises at least one of an inlet channel and outlet channel positioned lateral to the porous layer. In some aspects, the tissue sample includes at least one of an epidermis layer, a dermis layer, and a hypodermis layer. In some aspects, the porous layer includes a porosity gradient. In some aspects, the porous layer includes a plurality of pores, wherein each of the plurality of pores is a gyroid. In some aspects, the ex vivo skin tissue system further comprises at least one standoff along the second side of the porous layer. In some aspects, an interior surface of the wall perpendicular to the first side of the porous layer includes a plurality of pores. In some aspects, each of the plurality of pores is a gyroid. In some aspects, the plurality of pores penetrate a desired thickness of the wall. In some aspects, the ex vivo skin tissue system further comprises a gel layer in contact with the second side, wherein the gel layer is configured to receive one or more of adipose-derived cells, dermal cells, and endothelial cells. In some aspects, the gel layer includes a gel material. In some aspects, the gel layer includes extracellular matrix. In some aspects, the ex vivo skin tissue system further comprises an adhesive film configured for at least one of contacting and sealing the adipose layer. In some aspects, the ex vivo skin tissue system further comprises growth media in contact with the tissue sample. In some aspects, the growth media is maintained at between 32-37 degrees Celsius. In some aspects, the growth media includes at least one test agent. In some aspects, the ex vivo skin tissue system further comprises at least one test agent contacted to the tissue sample, wherein the contacting is one or more of topical application, subcutaneous application, systemic application, intradermal administration, infusion, perfusion, and injection.

In some aspects, provided herein is a method comprising: providing an insert including a porous layer, wherein the porous layer includes a structure for promoting mass transport of a fluid; arranging and sealing a skin tissue sample along an inner wall of the insert and relative to a first side of the porous layer so as to promote mass transport into the skin tissue sample; culturing the skin tissue sample, thereby making an ex vivo skin model; and perfusing reagents through the ex vivo skin model so as to support viability of the skin tissue sample for a time period of at least one week. In some aspects, the method further comprises depositing an adipose layer onto one or more of the first side, an internal region, and a second side of the porous layer. In some aspects, the porous layer is configured to promote vascularization of the tissue sample. In some aspects, the method further comprises seeding a cell composition within the porous layer between the first side and the second side. In some aspects, the seeding is performed using one or more of bioprinting, manual or automated liquid handling, and perfusion. In some aspects, the cell composition includes at least one of: endothelial cells, primary cells, iPSCs, keratinocytes, fibroblasts, melanocytes, resident immune cells, circulating immune cells, stem cells, pre-adipocytes and adipocytes. In some aspects, the resident immune cells include at least one of Langerhans cells, dendritic cells, macrophages, mast cells, monocytes, and lymphocytes. In some aspects, the circulating immune cells include at least one of neutrophils. T lymphocytes. B lymphocytes. natural killer cells, monocytes, and dendritic cells. In some aspects, the reagents include at least one of: media, non-essential amino acid solution, insulin, transferrin, selenous acid, bovine serum albumin, linoleic acid, hydrocortisone, Vitamin C, serum, blood substitute, penicillin and streptomycin. In some aspects, the method further comprises contacting the second side with a gel layer, wherein the gel layer is configured to receive at least one of adipose-derived cells, dermal cells, and endothelial cells. In some aspects, the gel layer includes a gel material. In some aspects, the gel layer includes extracellular matrix. In some aspects, the gel layer is bioprinted. In some aspects, the porous layer is in contact with at least one of the adipose layer and the tissue sample. In some aspects, the method further comprises at least one of an inlet channel and outlet channel positioned lateral the porous layer. In some aspects, the tissue sample includes at least one of an epidermis layer, a dermis layer, and a hypodermis layer. In some aspects, the porous layer includes a porosity gradient. In some aspects, the porous layer includes a plurality of pores, wherein each of the plurality of pores is in a shape of one or more of a gyroid, a Schwarz, an x-cell, and a cross.

In some aspects, the method further comprises at least one standoff along the second side of the porous layer. In some aspects, an interior surface of a wall perpendicular to the first side of the porous layer includes a plurality of pores. In some aspects, each of the plurality of pores is a gyroid. In some aspects, the plurality of pores penetrate a desired thickness of the inner wall. In some aspects, the method further comprises a gel layer in contact with the second side, wherein the gel layer is configured to receive at least one of the adipose layer and the tissue sample. In some aspects, the gel layer includes a gel material. In some aspects, the gel layer includes extracellular matrix. In some aspects, the sealing includes a compression unit, wherein the compression unit includes a microchannel. In some aspects, the compression unit includes an injection port in fluid communication with the microchannel. In some aspects, the method further comprises sealing the adipose layer with a gas-permeable film. In some aspects, an oscillating pressure is applied to the fluid to promote filling and draining of the fluid. In some aspects, the method further comprises: perfusing a composition through the insert; culturing the tissue sample; one or more of applying and monitoring environmental conditions; providing at least one sensor to obtain at least one functional readout; monitoring at least one of cell characteristics and tissue characteristics using the at least one sensor; and scoring the at least one of cell characteristics and tissue characteristics. In some aspects, one or more of the culturing the tissue sample and the monitoring of the at least one cell characteristics and tissue using the at least one sensor is performed for at least two weeks. In some aspects, the composition is adapted for maintaining and/or promoting a disease state. In some aspects, the composition is adapted for promoting cell differentiation. In some aspects, the composition is adapted for one or more of: promoting tissue complexity, maintaining multiple cell types, and developing cell-cell and/or cell-tissue interaction. In some aspects, the environmental conditions include at least one of: temperature, humidity, gas composition, pollution, and light exposure. In some aspects, the monitoring at least one of cell characteristics and tissue characteristics includes monitoring at least one of: irritation, corrosion, and phototoxicity. In some aspects, the monitoring at least one of cell characteristics and tissue characteristics includes monitoring at least one of: anti-aging, skin hydration, brightening, discoloration, pigmentation. UV protection, cleansing, permeability, inflammation, anti-inflammation, antimicrobial, wound healing, skin barrier, epidermal thickness, dermal matrix, cellular stress, cellular senescence, sagging, and wrinkling. In some aspects, each of the at least one sensor includes at least one clinical sensor. In some aspects, each of the at least one sensor includes: a viscoelasticity sensor, a hydration sensor, pH sensor, an oil content sensor, a barrier function sensor, a pigmentation sensor, and a skin surface sensor. In some aspects, the scoring includes calculating a percent change in the respective characteristic from a normal or vehicle condition. In some aspects, the scoring includes characterizing a method used to obtain the characteristic. In some aspects, an oscillating pressure is applied to the perfused composition to promote filling and draining of the perfused composition.

In some aspects, the techniques described herein relate to a method for skin tissue analysis, the method including: receiving a plurality of ex vivo skin tissue systems as described herein; mapping the plurality of datasets based on a scoring panel result, wherein the scoring panel result is calculated according to an intensity and effectiveness of each measurement of the mapped plurality of datasets; and generating at least one threshold criteria for identifying an agent from the at least one test agent based on the mapped plurality of datasets, wherein each of the at least one threshold criteria corresponds to at least one biological pathway. In some aspects, the skin tissue array includes one or more of an epidermal tissue layer and a dermal tissue layer. In some aspects, the skin tissue array includes one or more tissue layers from a biopsy, donor, or graft. In some aspects, the skin tissue array includes an adipose layer, wherein the adipose layer is beneath the porous layer. In some aspects, the skin tissue array includes an adipose layer, wherein the adipose layer is within the porous layer. In some aspects, the plurality of datasets include measurements from one or more of: skin surface, histology staining, immunostaining, biochemistry assay, clinical sensors, gene expression, and protein expression. In some aspects, measurements generated from the skin surface include measurements from one or more of: dermoscopy, mexameter, cutometer, and photography. In some aspects, measurements generated from the histology staining include measurements from one or more of: hematoxylin & eosin (H&E). Masson's trichrome, and Movat pentachrome. In some aspects, measurements generated from one or more of the gene expression and the protein expression include one or more of: dermal differentiation, dermal-epidermal junction, dermal marker, matrix metalloproteinases (MMPS). MMP inhibitors, regeneration, wrinkling. sagging, inflammation, intrinsic apoptosis, extrinsic apoptosis, anti-apoptosis, immune cell markers, skin elasticity, skin rejuvenation, and antioxidant defense. In some aspects, measurements generated from the clinical sensors include measurements from one or more of: moisture, barrier function, sebum, pH, dermoscopy, photography, melanin content, and ultrasound. In some aspects, the at least one threshold criteria are either positively correlated or negatively correlated with a condition. In some aspects, the condition includes one or more of: anti-aging, skin hydration, toxicity, brightening, discoloration, pigmentation, UV protection, cleansing, permeability, inflammation, anti-inflammation, antimicrobial, wound healing, skin barrier, epidermal thickness, dermal matrix, cellular stress, cellular senescence, sagging, and wrinkling. In some aspects, the skin tissue array is viable for longer than a week, In some aspects, the at least one biological pathway includes one or more of: skin surface, barrier function, epidermis thickness, matrix density, epidermis differentiation, dermal-epidermis junction, dermal papillary thickness and ridges, stem cell and regenerative activity, adipogenesis, melanogenesis, sagging pathways, wrinkling pathways, cellular senescence, cellular stress, inflammation, and apoptosis. In some aspects, the at least one threshold criteria are generated by applying a machine learning algorithm to the mapped plurality of datasets. In some aspects, the machine learning algorithm is further configured to perform one or more of: substantiating and generating product claims for skin concerns, performing broad efficacy and/or toxicity screening of chemical libraries for quantitative comparison to product benchmarks, and running virtual screens of the at least one test agent. In some aspects, the machine learning algorithm is either an unsupervised algorithm or a supervised algorithm. In some aspects, the porous layer is configured to promote mass transport into each tissue sample in the skin tissue array. In some aspects, the porous layer includes a porosity gradient. In some aspects, the porous layer includes a plurality of pores, wherein each of the plurality of pores is in a shape of one or more of a gyroid, a Schwarz, an x-cell, and a cross. In some aspects, the method further comprises contacting the skin tissue array with the at least one test agent, wherein the contacting is one or more of topical application, subcutaneous application, systemic application, intradermal administration, infusion, perfusion, and injection.

In some aspects, the techniques described herein relate to a method comprising: providing any of the ex vivo skin tissue systems as described herein; perfusing a composition through the ex vivo skin tissue system; culturing the ex vivo skin tissue system; one or more of applying and monitoring environmental conditions; providing at least one sensor; monitoring at least one of cell characteristics and tissue characteristics using the at least one sensor; and scoring the at least one of cell characteristics and tissue characteristics. In some aspects, one or more of the culturing and the monitoring is performed for at least two weeks. In some aspects, the composition is adapted for maintaining and/or promoting a disease state. In some aspects, the composition is adapted for promoting cell differentiation. In some aspects, the composition is adapted for one or more of: promoting tissue complexity, maintaining multiple cell types, and developing cell-cell and/or cell-tissue interaction. In some aspects, the environmental conditions include at least one of: temperature, humidity, gas composition, pollution, and light exposure. In some aspects, the monitoring at least one of cell characteristics and tissue characteristics includes monitoring at least one of: irritation, corrosion. phototoxicity, viability and metabolic activity. In some aspects, the monitoring at least one of cell characteristics and tissue characteristics includes monitoring at least one of: anti-aging, skin hydration, brightening, discoloration, pigmentation. UV protection, cleansing, permeability, inflammation, anti-inflammation, antimicrobial, wound healing, skin barrier, epidermal thickness, dermal matrix, cellular stress, cellular senescence, sagging, and wrinkling. In some aspects, each of the at least one sensor includes: a viscoelasticity sensor, a hydration sensor, pH sensor, an oil content sensor, a barrier function sensor, a pigmentation sensor, and a skin surface sensor. In some aspects, the scoring includes calculating a percent change in the respective characteristic from a normal or vehicle condition. In some aspects, the scoring includes characterizing a method used to obtain the characteristic. In some aspects, an oscillating pressure is applied to the perfused composition to promote filling and draining of the perfused composition.

In some aspects, the techniques described herein relate to an ex vivo skin tissue system including one or more inserts, wherein each of the one or more inserts includes: a drop reservoir at a proximal end; a basal reservoir at a distal end; a via sandwiched between the drop reservoir and the basal reservoir; and a tissue sample positioned inside at least one of the drop reservoir and the basal reservoir; wherein the via is configured to promote one or more of mass transport and cell growth from seeded cells into the tissue sample, wherein the via has a first side facing an opening of the drop reservoir and a second side facing an opening of the basal reservoir, and wherein the ex vivo skin tissue system is capable of supporting tissue viability for longer than a week. In some aspects, the ex vivo skin tissue system further comprises an adipose layer positioned inside at least one of the drop reservoir and the basal reservoir. In some aspects, the via is in contact with at least one of the adipose layer and the tissue sample. In some aspects, the tissue sample includes at least one of an epidermis layer, a dermis layer, and a hypodermis layer. In some aspects, the via includes a porosity gradient. In some aspects, the via includes porous layer, wherein the porous layer includes a plurality of pores. In some aspects, each of the plurality of pores is in a shape of one or more of a gyroid, a Schwarz, an x-cell, and a cross. In some aspects, the ex vivo skin tissue system further comprises a gel layer in contact with the second side, wherein the gel layer is configured to receive at least one of adipose-derived cells, dermal cells, and endothelial cells. In some aspects, the gel layer includes a synthetic gel material. In some aspects, the gel layer includes extracellular matrix. In some aspects, the seeded cells include one or more of primary cells, iPSCs, keratinocytes, fibroblasts, melanocytes, endothelial cells, resident immune cells, circulating immune cells, stem cells, adipocytes, and microbes. In some aspects, the drop reservoir, basal reservoir, and via are dimensioned such that a Bond number <1. In some aspects, the via includes an axially straight inner wall. In some aspects, the ex vivo skin tissue system further comprises growth media in contact with the tissue sample. In some aspects, the growth media is maintained at between 32-37 degrees Celsius. In some aspects, the growth media includes at least one test agent. In some aspects, the ex vivo skin tissue system further comprises at least one test agent in contact with the drop reservoir.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-1G are cross-sectional views of the ex vivo skin tissue system upon assembly of the tissue sample and adipose layer contacting a porous layer in the insert. FIG. 1A provides a cross-sectional view of an ex vivo skin tissue system that can include one or more of a compression unit, a tissue sample, a porous layer, an adipose layer, and an adhesive film. FIG. 1B provides a cross-sectional view of an ex vivo skin tissue system that can include one or more of an insert, a seeded adipose layer, a 3D printed porous structure, a gas permeable film, a tissue biopsy, a humidity control element, a nutrition delivery element, and optional endothelial cell seeding. FIGS. 1C-1G provide representations and photographs of a perfusion apparatus according to one or more aspects of the present disclosure. FIG. 1C provides a representation of a side view of a perfusion apparatus. FIG. 1D provides a representation of a perfusion apparatus that includes a reservoir container according to one or more aspects of the present disclosure. FIG. 1E provides a photograph showing a top-down view of a perfusion apparatus according to one or more aspects of the present disclosure. FIGURE IF provides a photograph showing a side view of a perfusion apparatus according to one or more aspects of the present disclosure. FIG. 1G provides a photograph showing a perfusion apparatus and perfusion controller according to one or more aspects of the present disclosure.

FIGS. 2A-2C are cross-sectional views of the insert. FIGS. 2A-2B illustrate embodiments wherein the pores are limited to the porous layer or extend along an inner surface of the insert. FIG. 2A illustrates an embodiment of an ex vivo skin tissue system that can include one or more of a porous structure, a gas permeable film, and a porous structure along insert walls. FIG. 2B illustrates an embodiment of an ex vivo skin tissue system that can include one or more of a lip, a porous structure, threading for a compression nut, and one or more standoffs. FIG. 2C illustrates an embodiment wherein the insert has a smaller outer diameter.

FIGS. 3A-3B are workflows of preparing and analyzing an ex vivo skin tissue system. FIG. 3A provides a flow chart illustrating a process for collecting data, including the controlling and/or monitoring of relevant biological and environmental conditions. FIG. 3B provides a flow chart illustrating a potential system for experimentation.

FIGS. 4A-4J are exemplary embodiments of the porous layer, according to aspects of this disclosure. FIGS. 4A-4D illustrate exemplary embodiments wherein the porous layer comprises a gyroid structure. FIG. 4A illustrates an embodiment of an ex vivo skin tissue system. FIG. 4B provides a photograph of a skin tissue model insert. FIG. 4C provides a photograph depicting an alternate view of a skin tissue model insert. FIG. 4D provides a photograph depicting an alternate view of a skin tissue model insert. FIGS. 4E-4J illustrate additional exemplary embodiments wherein the porous layer comprises microneedles or mesh. FIG. 4E provides a photograph of a custom skin tissue model insert with microneedles. FIG. 4F provides a photograph of an alternate view of a custom skin tissue model insert with hollow microneedles in fluid communication with a perfusable microchannel. FIG. 4G provides a photograph of an alternate view of a custom skin tissue model insert with microneedles. FIG. 4H provides a photograph of an alternate view of a custom skin tissue model insert with microneedles. FIG. 4I provides a photograph of a mesh structure for a skin tissue culture insert. FIG. 4J provides a photograph of an alternate view of a mesh structure for a tissue culture insert.

FIGS. 5A-5B are cross-sectional and perspective views, respectively, of an exemplary embodiment of the compression nut. FIG. 5A provides a cross-sectional view of a model of a compression nut for holding a tissue sample, including a microchannel. FIG. 5B provides a perspective view of a model of a compression nut for holding a tissue sample, including an injection port.

FIGS. 6A-6D are various views of a recirculation system according to several embodiments of this disclosure. FIG. 6A provides a view of a tissue culture modelling system that can include one or more of an insert for holding a tissue sample, an insert cap, a recirculation system, a reservoir cap, a fluid capacitor, and a media reservoir. FIG. 6B provides a view of a tissue culture modelling system that can include integrated check valves. FIG. 6C provides a view of a tissue culture modelling system that can include one or more of a fluid inlet, a fluid outlet, and a membrane. FIG. 6D provides a view of a model of an ex vivo skin tissue system and media reservoir with recirculating pump. FIG. 6E provides a view of a model of an ex vivo skin tissue system with multiple culture inserts, with an inset diagram of the structure of human skin.

FIGS. 7A-7G are a perspective and cross-sectional view, respectively, of a high-throughput cartridge (HTC) system. FIG. 7A provides a perspective view of a skin tissue modelling system compatible with a SBS microtiter plate. FIG. 7B provides a cross-sectional view of a skin tissue modelling system that can include one or more of an insert, a media reservoir, and a lid. FIGS. 7C-7F illustrate additional embodiments of the HTC system, according to some aspects of this disclosure. FIG. 7C provides a view of a skin tissue modelling system that can include one or more of a drop reservoir, a via, and a basal reservoir. FIG. 7D provides a view of a model of a skin tissue modelling system. FIG. 7E provides a view of a model of a skin tissue culture modelling system. FIG. 7F provides a view of a model of a skin tissue culture modelling system. FIG. 7G provides a close-up view of a single insert in the HTC system, according to some aspects of this disclosure.

FIGS. 8A-8C are diagrams illustrating a data extraction workflow and a diagram describing a workflow for data extraction, data mapping, and claim quantification, respectively, according to one or more aspects of the present disclosure.

FIG. 9 is a block diagram of a system with which some embodiments may operate.

FIG. 10 is a block diagram of a computing device with which some embodiments may operate.

FIGS. 11A-11F are a series of micrographs for tissue samples subjected to test conditions with different stains applied. FIG. 11A-11B provides micrographs with hematoxylin-eosin stain. FIG. 11C-11D provides micrographs with Masson's Trichrome stain. FIG. 11E-11F provides micrographs with Movat Pentachrome stain.

FIGS. 12A-12F are gene expression maps for several different study conditions according to one or more aspects of the present disclosure. FIG. 12A provides gene expression maps for epidermal differentiation and dermal-epidermal junction. FIG. 12B provides gene expression maps for dermal markers and MMPs. FIG. 12C provides gene expression maps for MMP inhibitors and regeneration. FIG. 12D provides gene expression maps for wrinkle-related genes and sagging-related genes. FIG. 12E provides gene expression maps for and inflammation and apoptosis (intrinsic). FIG. 12F provides gene expression maps for apoptosis (extrinsic) and anti-apoptotic genes.

FIGS. 13A-13B are micrographs for stained tissue samples according to one or more aspects of the present disclosure. FIG. 13A provides a histology stained skin tissue sample at day zero of culture. FIG. 13B provides a histology stained skin tissue sample after three weeks of culture.

FIG. 14 is a gene expression map showing expression data for genes involved in skin development and function across several samples according to one or more aspects of the present disclosure.

FIGS. 15A-15G are micrographs for histology stained skin tissue samples according to one or more aspects of the present disclosure. FIG. 15A provides a micrograph showing a histology stained skin tissue culture with Media Mix 1 of Table 6. FIG. 15B provides a micrograph showing a histology stained skin tissue culture with Media Mix 3 of Table 6. FIG. 15C provides a micrograph showing a histology stained skin tissue culture with Media Mix 6 of Table 6. FIG. 15D provides a micrograph showing a histology stained skin tissue culture with Media Mix 8 of Table 6. FIG. 15E provides a micrograph showing a histology stained skin tissue culture with Media Mix 9 of Table 6. FIG. 15F provides a micrograph showing a histology stained skin tissue culture with Media Mix 11 of Table 6. FIG. 15G provides a micrograph showing a histology stained skin tissue culture with Media Mix 12 of Table 6.

FIGS. 16A-16B are micrographs showing histology stained skin tissue samples according to one or more aspects of the present disclosure. FIG. 13A provides a micrograph showing histology stained skin tissue culture after day zero of culture with Media Mix 12 of Table 6. FIG. 16B provides a micrograph showing histology stained skin tissue culture after four weeks of tissue culture with Media Mix 12 of Table 6.

FIGS. 17A-17C are photographs of skin surface of skin tissue culture after six weeks of culture according to one or more aspects of the present disclosure. FIG. 17A provides a photograph of skin tissue surface after six weeks of culture in Media Mix 12 of Table 6. FIG. 17B provides a photograph of skin tissue culture surface after six weeks of culture in Media Mix 12 of Table 6 with 5% human platelet lysate. FIG. 17C provides a photograph of human skin tissue culture surface after six weeks of culture in Media Mix 12 of Table 6 with 5% human platelet lysate and EGF at 10 nanograms per milliliter and bFGF at 10 nanograms per milliliter.

FIGS. 18A-18B are a photograph and a micrograph of skin biopsies sealed in tissue culture inserts according to one or more aspects of the present disclosure. FIG. 18A provides a photograph of three 20 mm sealed biopsies. FIG. 18B provides a micrograph of an H&E-stained cultured skin tissue biopsy adhered to a skin tissue culture insert.

FIGS. 19A-19B are graphs of TEWL measurements for both sealed and unsealed biopsies in tissue inserts according to one or more aspects of the present disclosure. FIG. 19A provides a graph of TEWL time course measurements in skin biopsies contained in tissue culture inserts without sealing. FIG. 19B provides a graph of TEWL time course measurements in skin biopsies sealed to tissue culture inserts.

FIG. 20 provides a graph of TEWL measurements over a six-day time course in skin tissue biopsy in sealed tissue culture inserts according to one or more aspects of the present disclosure.

FIG. 21A provides a representation of the incorporation of raw multi-modal data into a data map according to one or more aspects of the present disclosure. FIGS. 21B-21C provide representative illustrations of data maps corresponding to compounds of interest according to one or more aspects of the present disclosure.

FIGS. 22A-22B provide a photograph and a micrograph of a 3D printed gyroid support structure according to one or more aspects of the present disclosure. FIG. 22A provides a photograph of a 3D-printed gyroid support structure with endothelial cell network stained with a CD31 endothelial cell marker. FIG. 22B provides a micrograph showing a detailed view of a 3D-printed gyroid support structure with endothelial cell network stained with a CD31 endothelial cell marker.

FIGS. 23A-23H provide micrographs of cell networks after 14 days of tissue culture in CELLnTEC-Promocell Media Mix and CD31 staining according to one or more aspects of the present disclosure. FIG. 23A provides a micrograph showing cells stained with ObaGel™ ECM. FIG. 23B provides a micrograph showing cells stained with ObaGel™ Original. FIG. 23C provides a micrograph showing cells cultured with Fibrinogen (2 mg/mL), Aprotinin (1 ug/mL), Geltrex™ (40 ug/ml) and Thrombin* (0.5 U/mL). FIG. 23D provides a micrograph showing cells cultured with Fibrinogen (2 mg/mL), Aprotinin (5 ug/mL), Geltrex™ (40 ug/ml), and Thrombin* (0.5 U/mL). FIG. 23E provides a micrograph showing cells cultured with a 2D coating of: Collagen IV (400ug/mL), FN (100 ug/mL), and Geltrex (50 ug/mL). FIG. 23F provides a micrograph showing cells cultured with a 2D coating of: Collagen IV (200 ug/mL), FN (50 ug/mL), and Geltrex™ (50) ug/mL). FIG. 23G provides a micrograph of cells cultured with a 2D coating of: Collagen I (100 ug/mL), Collagen IV (200 ug/mL,) FN (50 ug/mL), Geltrex™ (50 ) ug/mL). FIG. 23H provides a micrograph showing the cell-only control group.

FIGS. 24A-24H provide micrographs of cells after endothelial growth media pre-treatment (8 days) followed by CELLnTEC-Promocell Media Mix (6 days) and CD31 staining according to one or more aspects of the present disclosure. FIG. 24A provides a micrograph showing cells stained with ObaGel™ ECM. FIG. 24B provides a micrograph showing cells stained with ObaGel™ Original. FIG. 24C provides a micrograph showing cells cultured with Fibrinogen (2 mg/mL), Aprotinin (1 ug/mL), Geltrex™ (40 ug/ml) and Thrombin* (0.5 U/mL). FIG. 24D provides a micrograph showing cells cultured with Fibrinogen (2 mg/mL), Aprotinin (5 ug/mL), Geltrex™ (40 ug/ml), and Thrombin* (0.5 U/mL). FIG. 24E provides a micrograph showing cells cultured with a 2D coating of: Collagen IV (400 ug/mL), FN (100 ug/mL), and Geltrex (50 ug/mL). FIG. 24F provides a micrograph showing cells cultured with a 2D coating of: Collagen IV (200 ug/mL), FN (50) ug/mL), and Geltrex™ (50) ug/mL). FIG. 24G provides a micrograph of cells cultured with a 2D coating of: Collagen I (100 ug/mL), Collagen IV (200 ug/mL.) FN (50) ug/mL), Geltrex™ (50 ug/mL). FIG. 24H provides a micrograph showing the cell-only control group.

FIG. 25 provides a micrograph showing adipose-derived stem cells with Media Mix 12 of Table 6 and stained with F-actin according to one or more aspects of the present disclosure.

FIGS. 26A-26C are representations showing skin tissue subjected to clinically relevant changes according to one or more aspects of the present disclosure. FIG. 26A provides a representation showing topical chemical treatment of skin tissue. FIG. 26B provides a representation showing skin tissue subjected to contact with a heated metal rod. FIG. 26C provides a representation showing skin tissue subjected to UV treatment.

FIG. 27 is a representation of a treatment schedule for replicating dynamic changes in skin across various types of clinically relevant stimuli according to one or more aspects of the present disclosure.

FIGS. 28A-28D are micrographs showing histology stained skin tissue samples according to one or more aspects of the present disclosure. FIG. 28A provides a micrograph showing untreated skin tissue. FIG. 28B provides a micrograph showing SDS-irritated skin tissue. FIG. 28C provides a micrograph showing burned skin tissue. FIG. 28D provides a micrograph showing skin tissue subjected to UV irradiation.

FIGS. 29A-29C are graphs showing histopathological scoring in different groups of cells subjected to clinically relevant stimuli according to one or more aspects of the present disclosure. FIG. 29A provides a graph showing epidermal damage in skin tissue culture samples subjected to UV radiation. SDS, heated metal rod, and samples without treatment. FIG. 29B provides a graph showing eczematous epidermis in skin tissue culture samples subjected to UV radiation. SDS, heated metal rod, and samples without treatment. FIG. 29C provides a graph showing thickness of epidermis in skin tissue culture samples subjected to UV radiation. SDS, heated metal rod, and samples without treatment.

FIGS. 30A-30D are micrographs of histology stained skin tissue samples according to one or more aspects of the present disclosure. FIG. 30A provides a micrograph showing untreated skin tissue cultures. FIG. 30B provides a micrograph showing skin tissue cultures subjected to SDS irritation. FIG. 30C provides a micrograph showing skin tissue cultures subjected to burn wounds. FIG. 30D provides a micrograph showing skin tissue cultures subjected to UV radiation.

FIGS. 31A-31B are graphs showing histopathological scoring of collagen deposition and lactate dehydrogenase (LDH) release, respectively, in skin tissue culture samples subjected to UV radiation. SDS, heated metal rod, and samples without treatment according to one or more aspects of the present disclosure.

FIGS. 32A-32B are expression heatmaps showing the immune response of skin tissue cultures in response to clinically relevant stimuli according to one or more aspects of the present disclosure.

FIGS. 33A-33C are graphs showing the inflammatory response of skin in response to clinically relevant stimuli according to one or more aspects of the present disclosure. FIG. 33A provides a graph showing IL-1β concentration (pg/mL) in baseline (untreated), SDS-treated, burned, and UV-treated skin tissue culture samples. FIG. 33B provides a graph showing IL-6 concentration (pg/mL) in baseline (untreated). SDS-treated, burned, and UV-treated skin tissue culture samples. FIG. 33C provides a graph showing IL-8 concentration (pg/mL) in baseline (untreated), SDS-treated, burned, and UV-treated skin tissue culture samples.

FIGS. 34A-34C are a representation, a micrograph, and a graph of intradermal injection in skin tissue culture according to one or more aspects of the present disclosure. FIG. 34A provides a representation of a skin tissue culture sample subjected to intradermal injection. FIG. 34B provides a micrograph of stained, intradermally injected skin tissue culture samples. FIG. 34C provides a graph of IL-1β concentration (pg/mL) in samples treated with saline. LPS 1 μg/mL. LPS 10 μg/mL, and LPS 10 μg/mL with TNF-α.

FIGS. 35A-35B are expression heatmaps of immune cell profiling of two different donors in connection with relevant compounds according to one or more aspects of the present disclosure.

FIGS. 36A-36B are an expression heatmap and a graph of Superoxide dismutase (SOD activity showing antioxidant defense response according to one or more aspects of the present disclosure. FIG. 36A provides an expression heatmap showing antioxidant-related gene expression associated with treatment with several skincare ingredients. FIG. 36B provides a graph showing SOD activity from several UV-treated samples.

FIG. 37 is an exemplary embodiment of a diagram of a configuration of a perfusion controller relative to an ex vivo skin tissue system according to one or more aspects of the present disclosure.

### DETAILED DESCRIPTION

Described herein are systems and techniques for compound or biologic screening with increased tissue complexity and longevity. This disclosure relates generally to ex vivo skin tissue models, also referred herein as ex vivo skin tissue models, and support systems therefor. such that the ex vivo skin tissue models may be allowed to promote and maintain advanced features such as enhanced longevity, skin-like tissue structure, functionality, and immunocompetency for extensive analytical outcomes, for instance, efficacy and/or toxicity testing. In some aspects, the methods described herein include analyzing skin tissue during one or more of tissue growth and development or responses to compounds or biologics of interest. In some aspects, the methods of testing compositions comprises selecting therapeutic agents. In some aspects the methods of testing compositions use big data and diverse datasets generated, for example, from the ex vivo tissue support systems, and processed using machine learning algorithms as described herein to substantiate and generate evidence-based skincare product claims for skin concerns, perform broad efficacy and/or toxicity screening of chemical libraries for potential quantitative comparison to product benchmarks, and train artificial intelligence (AI) models to run virtual screens. In some aspects, this disclosure provides ex vivo tissue skin model systems comprising a sealing unit; a skin tissue; a porous structure configured to promote mass transport into one or more of a skin tissue biopsy, an endothelial layer, and an adipose layer, and interaction therebetween.

Provided herein are (1) insert and cartridge devices; (2) workflows and systems for engineered ex vivo skin tissue analysis; (3) porous layers for enhanced perfusion; (4) samples, cells and tissues for incorporation in such innovations; (5) high-content model systems; (6) high-throughput model systems; (7) methods of intelligent data analysis; (8) methods of data monitoring and scoring; and (9) systems and methods of computer implementation.

In some embodiments described here, ex vivo tissue support systems are curated in specific ways and estimates of efficacy of the system, and example active ingredients upon testing with the system are produced based on analyzing the estimates with a variety of standardized techniques. While lab-based experiments have been continuously used to evaluate the efficacy of active ingredients from developers on matrices such as skin tissue, a lack of scalable, physiologically relevant skin models in the market has led developers to utilize testing platforms that are limited in their ability to screen libraries and assess mechanistic insight.

### I. Insert and Cartridge Devices for Engineered Ex Vivo Skin Tissue Models

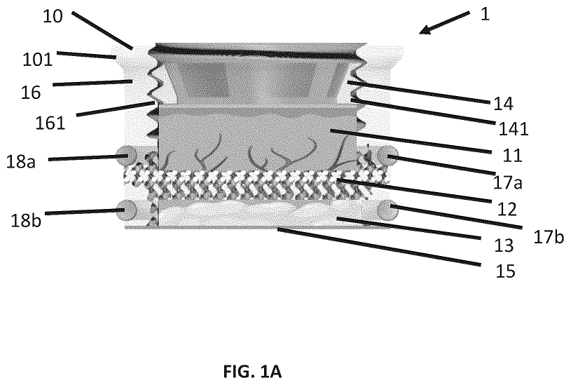

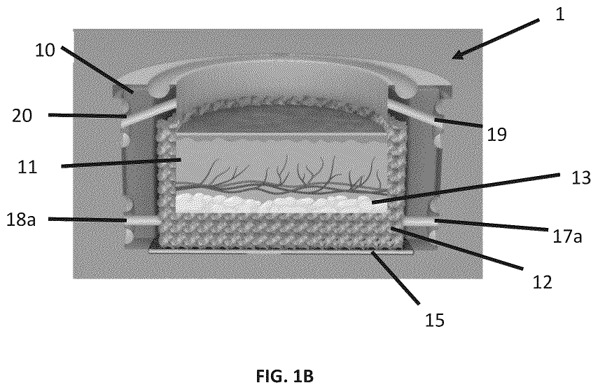

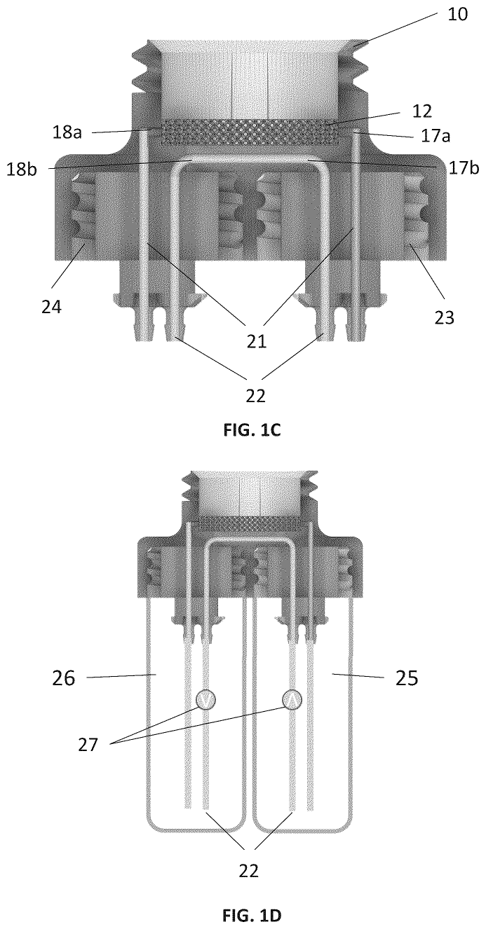

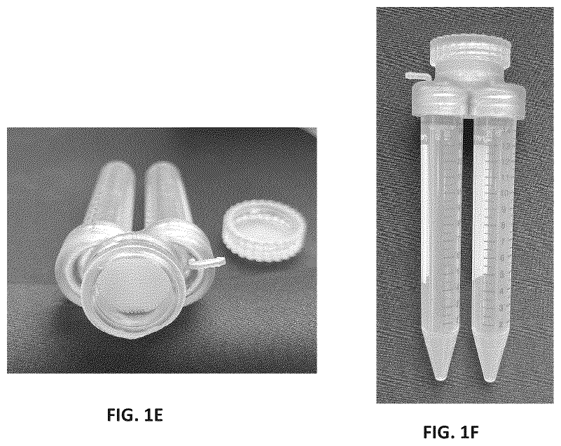

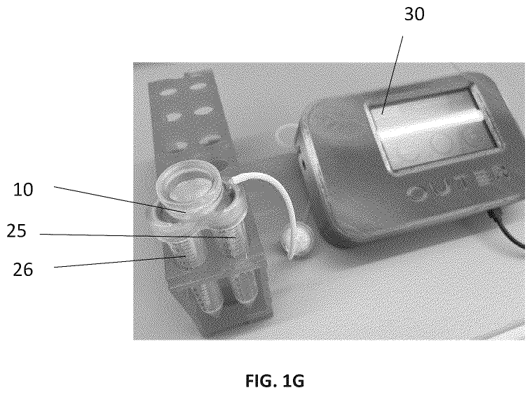

In various aspects, this disclosure provides ex vivo skin tissue systems that may be used as platforms for maintaining complex tissue models, which may be monitored in response to exposure to various test agents over extended time frames compared to contemporary test platforms. FIGS. 1A-1B illustrate the ex vivo skin tissue system according to exemplary embodiments of this disclosure. FIG. 1A illustrates an exemplary embodiment wherein an adipose layer is deposited on the second side of the porous layer, whereas FIG. 1B illustrates an exemplary embodiment wherein an adipose layer is deposited on the first side of the porous layer.

In some aspects, the ex vivo skin tissue system I comprises an insert 10, the insert 10 comprising compartments for skin tissue growth comprising at least one skin tissue sample 11, a porous layer 12, and, optionally, an adipose tissue layer 13, a compression unit 14, and a film 15. In certain embodiments, the insert 10 may be referred to as a chamber or enclosure. In some aspects, the at least one skin tissue sample 11 comprises at least one skin tissue layer. In some aspects, the at least one skin tissue layer comprises at least one of an epidermal tissue layer, a dermal tissue layer, and a hypodermal tissue layer. In some aspects, the at least one skin tissue system 11 comprises one or more of: a stratum corneum, a granular cell layer, a spinous cell layer, a basal cell layer, a sebaceous gland, an erector pili muscle, a sweat gland, nerves, a hair follicle, collagen and elastin fibers or connective tissue, blood vessels (e.g., an artery and/or a vein), and fat (e.g., adipose) tissue. In some aspects, the porous layer 12 comprises a structure with openings sufficient for vascularization from the adipose tissue layer into the epidermal and/or dermal tissue layer. In some embodiments, the skin tissue system 11 is viable for longer than a week. Viability, as used herein, may be considered when a percentage of each of tissues and/or cultured cells is evaluated to be at least 80% alive. Viability may also be considered when there is less than a 20% change in functions such as, for example, cell proliferation, differentiation, migration, and cell-cell, cell-matrix, and immune cell interactions, as compared to a day 0 control skin tissue sample. In some embodiments, the skin tissue system 11 is viable a time period of at least two weeks. In some aspects, the ex vivo skin tissue system 1 comprises a seeded cell composition within the porous layer 12 between the first side and the second side. In some embodiments, the seeded cell composition comprises at least one of: primary cells. iPSCs, endothelial cells (e.g., microvascular dermal endothelial cells—HDMECs), dermal cells (e.g., fibroblasts), epidermal cells (e.g., keratinocytes and melanocytes), resident immune cells, circulating immune cells, stem cells, pre-adipocytes and adipocytes. In some embodiments, the resident immune cells comprise at least one of Langerhans cells. Merkel cells, dendritic cells, macrophages, mast cells, monocytes, and lymphocytes. In some embodiments, the circulating immune cells comprise at least one of neutrophils, T lymphocytes, B lymphocytes, natural killer cells, monocytes, and dendritic cells.

The porous layer 12 may provide a primary benefit of increasing mass transport into the tissue biopsy through a plurality of pores. In some embodiments, the insert 10 comprises a first threading 161 along an interior surface of a wall 16 perpendicular to the first side of the porous layer 12. In some embodiments, the first threading 161 has a pitch ranging from 1 mm to 0.5 mm. In some embodiments, the compression unit 14 comprises a second threading 141 along an outer perimeter configured for mating the first threading 161. The compression unit 14 is dimensioned to fixate the skin tissue sample 11 within the insert 10 at a desired applied pressure, based on the height at which the compression unit 14 is tightened within the insert 10. Inadequate pressure, e.g., the compression unit 14 being positioned further from the porous layer 12, causes the tissue to loosen from the insert 10, whereas excess pressure, e.g., the compression unit 14 being positioned closer to the porous layer 12, induces torque on the tissue during rotation, causing the tissue to balloon in the middle and reduce tightness.

In some embodiments, the insert 10 comprises at least one of an inlet channel 17a,b and outlet channel 18a,b positioned lateral the porous layer 12, such that perfusion of fluid through each of the inlet channel 17a,b and outlet channel 18a,b cause fluid to flow through the porous layer 12. In some embodiments, the fluid is applied via a pressure pump and/or a fluid capacitor. In some embodiments, an oscillating pressure is applied to the fluid. This promotes filling and draining of fluid in the ex vivo skin tissue system 1, such as, for example, filling and draining of vasculature in a tissue sample. In some embodiments, this is accomplished via an air over liquid pressure pumping system. In some embodiments, this is accomplished by changing a hydrostatic pressure in the ex vivo skin tissue system 1 by dynamic positioning of one or more of the inlet channel 17a,b and outlet channel 18a,b and/or the reservoirs connected thereto.

In some embodiments, the insert 10 comprises an upper inlet 19 and an upper outlet 20 for humidity control. In some embodiments, the insert 10 comprises a lip 101 along an outer edge of an opening(s) side of the insert 10. In some embodiments, an outer surface of the insert 10 comprises indents for fitting one or more O-ring, wherein the one or more O-ring is configured to seal the insert 10 within a cartridge perfusion system.

FIGS. 1C-1D illustrate exemplary embodiments of an ex vivo skin tissue system 1 adapted for establishing fluid communication with solutions for perfusion. FIG. 1C illustrates ex vivo skin tissue system I comprising a compartment for a biopsy of the skin tissue sample 11, a porous layer 12, a flow path 21 comprising an inlet 17a and outlet 18a in the porous layer 12, a recirculation path 22 comprising an inlet 17b and outlet 18b below the porous layer 12, and fittings 23,24 for at least a first reservoir 25 and a second reservoir 26. FIG. 1D illustrates the ex vivo skin tissue system 1 including extensions of the flow and recirculation paths 21,22 into reservoir containers, such as, for example, a conical tube. In some embodiments, the flow and recirculation paths 21,22 further include one or more check valve 27 for controlling flow. FIGS. 1E-1G are photographic images of the embodiment in FIGS. 1C-1D, specifically a top view; a side view; and a perspective view in connection with a perfusion controller 30, respectively. In some embodiments, the perfusion controller 30 comprises a user interface (UI) configured to transmit pressure setpoints and time durations to an air pump engaged with at least one of the first reservoir 25 and the second reservoir 26. The air pump may be configured to supply vacuum or positive pressure to either of the first or second reservoirs 25,26, thereby forcing fluid to flow through the ex vivo skin tissue system 1. In some embodiments, the perfusion controller 30 is configured to control one or more of a fluid perfusion rate, a fluid perfusion time period, a recirculation rate, and a recirculation frequency. In some embodiments, the perfusion controller 30 is configured to control an air perfusion rate and time period, such as on an epidermal side of the tissue sample 11, and/or a positive pressure to stretch or balloon the tissue sample 11.

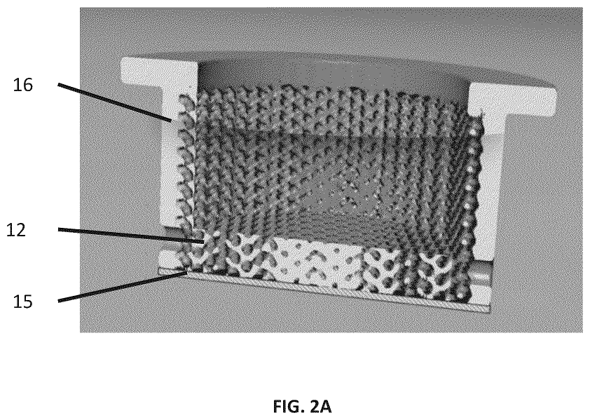

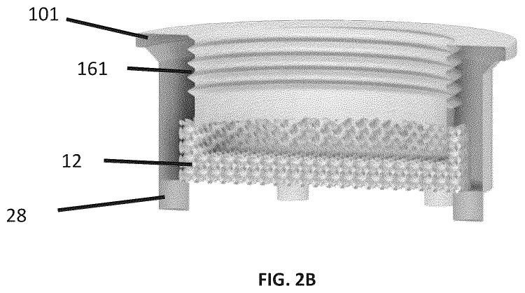

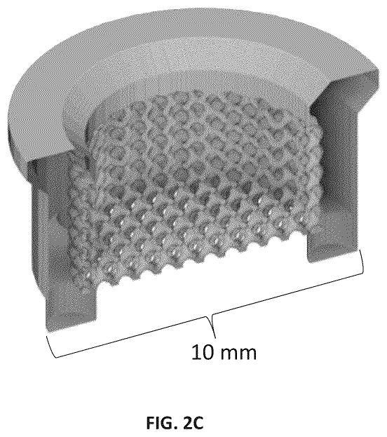

FIGS. 2A-2C illustrate cross-sectional view of the insert 10 according to exemplary embodiments of this disclosure. FIG. 2A illustrates an embodiment wherein a plurality of pores extend along an entirety of the inner wall 16 of the insert 10, whereas FIG. 2B illustrates an embodiment wherein a plurality of pores partially extend along the inner wall 16 of the insert 10. The plurality of pores along the inner wall 16 provide additional control over humidity within the insert 10. In some embodiments, the insert 10 is in a format that can be placed in an SBS microtiter plate similar to a TranswellR; insert. In some embodiments, the insert 10 is configured to plug into a cartridge, wherein the cartridge can fit a plurality of inserts.

In some embodiments, the insert 10 comprises at least one standoff 28 along the second side of the porous layer 12. The standoff 28 allows for media to enter the insert 10 from below the porous layer 12, which may remove the need for inlet channels and outlet channels for perfusion. A standoff 28 may comprise one or more cylindrical stand below the insert 10, as depicted in FIG. 2B. FIG. 2C illustrates an embodiment wherein an outer diameter of the insert 10 is 10 mm. In some embodiments, the insert 10 has an outer diameter ranging from 4 mm to 30 mm. In some embodiments, each of the inserts comprises a lip 101 circumferentially along a top surface.

### II. Workflows and Systems for Engineered Ex Vivo Skin Tissue Analysis

In various aspects, this disclosure provides methods of preparing and analyzing an ex vivo skin tissue system 1 with enhanced longevity. In vivo testing platforms, while critical to understanding complex biological processes, are not easily replicated and may be costly when pursuing big data sets. Alternatively, in vitro testing platforms can be done at significantly lower costs when seeking similar data quantities and in much more controlled environments, though are often less reflective of personalized outcomes. While both types of platforms are useful, ex vivo testing is arguably a better proxy for in vivo human skin by often allowing normal skin barrier function and support for layers critical for a variety of desirable skin tests such as the epidermis (e.g., stratum corneum, stratum lucidum, granular layer, spinous layer, basal layer, Langerhans cells, etc.), the dermis (papillary and reticular), the hypodermis (fat/adipose cells, blood vessels, bursa, etc.), and various skin appendages (e.g., sweat glands and hair follicles). However, tissue and cell viability diminish rapidly after excision, and most contemporary ex vivo platforms are lacking in support capabilities to ensure functionalities such as mechanical stability, immunocompetency, metabolic activity, and vascularization.

In contrast, ex vivo tissue support systems described herein are designed to address such unmet needs. The ex vivo tissue support systems are shaped to support tissue samples such as those from a biopsy punch, and are structured to facilitate perfusion through the biopsy that better mimics tissue support in live humans. Further, the ex vivo skin tissue system 1 described herein may be used as platforms for collecting data in contribution to a DAW described herein by providing potential molecular, genomics, and/or proteomics profiles of skin tissue samples resulting from test agents, for example, those in skincare products. The following process workflows may allow for consistency and reliability in preparing the ex vivo skin tissue system 1.

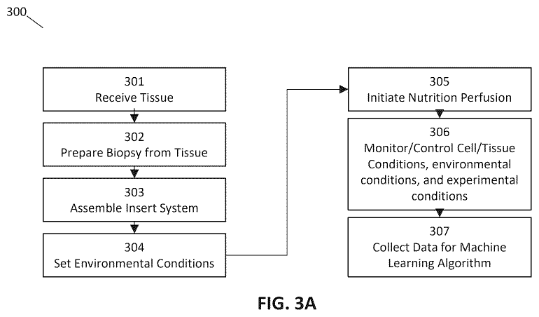

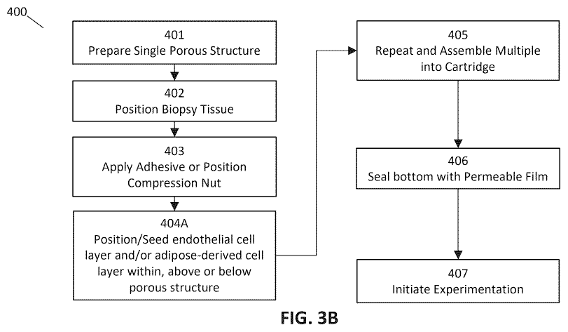

Referring to FIG. 3A an exemplary process workflow 300 is provided for preparing a model system consistent with this disclosure. In some embodiments, the methods described herein comprise preparing a plurality of model systems. In some embodiments, the methods described herein are as follows. Step 301 comprises receiving a tissue sample; step 302 comprises preparing a biopsy from the tissue sample; step 303 comprises assembling at least one of tissue and cells within an insert 10; step 304 comprises setting/controlling environmental conditions; step 305 comprises initiating and maintaining perfusion through the insert 10; step 306 comprises culturing the at least one of tissue and cells and/or monitoring the at least one of tissue and cell conditions and environmental conditions; step 307 comprises and collecting data for downstream analysis. In some embodiments, the biopsy preparation is performed manually or with a hydraulic/pneumatic press. In some embodiments, the method comprises tolerance checking of the ex vivo skin tissue system 1, cell and tissue conditions for monitoring. and environmental conditions for monitoring include humidity, dryness, temperature, gas control, pollution, perfusion rate. UV, etc. In some embodiments, the cell and tissue clinical measurements comprise those for one or more of viability, vascularization, aging. viscoelasticity, differentiation, maturation, and overall complexity. In some embodiments, the environmental conditions comprise one or more of humidity/dryness, temperature, gas (e.g., CO₂, O₂, etc.) exposure and control, pollution, and light (e.g., ultraviolet (UV)) exposure. The environmental conditions are discussed further below in reference to TABLE 1.

FIG. 3B provides an overview of methods for assembling the ex vivo skin tissue system 1. In some embodiments, methods described herein comprise process workflow 400, which comprises the following steps. Step 401 comprises preparing an insert 10 with a porous layer 12. In some embodiments, the porous layer 12 has a first side and second side, wherein the first side faces one opening(s) of the insert 10 and the second side faces an opposite direction of the opening(s) of the insert 10. Step 402 comprises positioning an epidermal and/or dermal biopsy within the insert 10. Step 403 comprises optionally applying a sealing unit within an inner wall 16 of the insert 10, wherein the inner wall 16 is adjacent and perpendicular to a first side of the porous layer 12. The sealing unit allows for sealing of the tissue sample within the insert 10. In some embodiments, the sealing unit comprises a compression unit 14 with threads for mating with the inner wall 16 of the insert 10. In some embodiments, the sealing unit is an adhesive. In some embodiments, the adhesive is selected from a medical grade super glue and a UV curable glue. Step 404 comprises optionally positioning or seeding an endothelial cell layer within the insert 10, such as within the porous structure of the insert 10. Step 404 may further comprise optionally positioning an adipose-derived cell layer within or below the porous structure of the insert 10. Step 405 comprises optionally repeating the steps 401-404 so as to assemble multiple inserts into a cartridge. Step 406 comprises optionally sealing a bottom of each insert 10 with a permeable film. Step 406 comprises initiating an experiment. embodiments of which are described further in detail herein.

In some embodiments, the methods described herein further comprise coating the insert 10 and porous layer 12 with a gel layer. In some embodiments, methods described herein further comprise contacting the second side with a gel layer, wherein the gel layer is configured to receive a seeded adipose layer. In some embodiments, the gel layer comprises a synthetic gel material. In some embodiments, the gel layer comprises extracellular matrix (ECM).

In some embodiments, the monitoring the at least one of tissue and cell conditions and environmental conditions comprises the use of at least one sensor. In some embodiments, each of the at least one sensor comprises: a viscoelasticity sensor, a hydration sensor, pH sensor, an oil content sensor, a barrier function sensor, a pigmentation sensor, and other skin surface sensor. In some embodiments, at least one sensor comprises a corneometer, a transepidermal water loss (TEWL) sensor, a melanin sensor, and an ultrasound sensor. In some embodiments, the sensors usable to detect such signals can include, for example, optical sensors (e.g., imaging devices for detecting fluorescence or optical signals with various wavelengths and frequencies), electric potential sensors, surface plasmon resonance (SPR) sensors, interferometric sensors, or any other type of sensor suitable for detecting calorimetric signals, potentiometric signals, amperometric signals, optical signals, or piezo-electric signals.

In some embodiments, the media comprises a composition adapted for maintaining and/or promoting a disease state. In some embodiments, a disease state may be maintained when a tissue sample comprises diseased tissue, such that its phenotype is maintained for a full culture as a control. In some embodiments, the disease state comprises at least one of inflammation, acne, dermatitis, psoriasis, rosacea, and eczema, hyperpigmentation. discoloration, and sun damage. In some embodiments, the disease state comprises at least one of fungal or bacterial infections, such as tinea pedis (athlete's foot), tinea curis (jock itch), tinea corporis (ringworm), cellulitis, erysipelas, and erythrasma. In some embodiments, the media comprises a composition adapted for promoting cell differentiation. In some embodiments, the media comprises a composition adapted for promoting tissue complexity. In some embodiments, the media comprises components and supplements comprising at least one of growth factors, vitamins, hormones, trace elements, and other micronutrients to support the cell type for an extended period of time. In some embodiments, the media comprises a composition for promoting microbiota growth.

Once the skin tissue has been developed, maintained and appropriately validated using measurements of its progress within the system, data of the conditions and effects of various test agents may be collected and aggregated for contribution within the DAW.

### III. Porous Layers for Enhanced Perfusion

In various aspects, this disclosure provides a porous layer 12 that may improve development of skin tissue sample complexity and longevity during the course of experimentation in collecting data for the DAW. In particular, a porous layer 12 as described herein may facilitate multiple advantages, such as, for example, increasing adhesion between the cells and/or tissue with an insert 10, maintain barrier function of a tissue sample, increasing mass transport of one or more of perfused fluid, nutrients, waste, and/or oxygen to or from a tissue sample, and promoting vascularization of the tissue sample within the ex vivo tissue support system. The porous layer 12 may further provide a foundation for direct seeding of cells during perfusion through the ex vivo tissue support system.

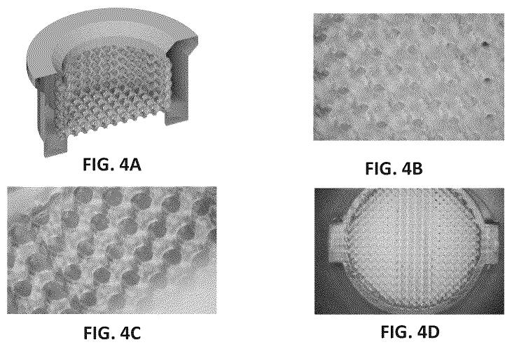

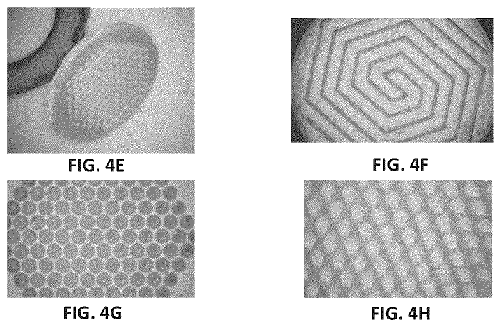

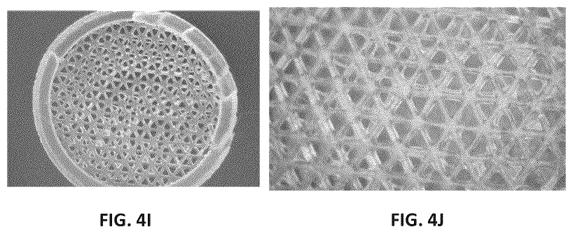

FIGS. 4A-4D illustrate exemplary embodiments of the porous layer 12, according to aspects of this disclosure. FIG. 4A illustrates a cross-sectional view of a 3D-rendering of the insert 10 with a porous layer 12. In some embodiments, an interior surface of a wall 16 perpendicular to the first side of the porous layer 12 comprises a plurality of pores. In some embodiments, each of the plurality of pores is a gyroid. In some embodiments, the plurality of pores penetrates a desired thickness of the wall 16. In some embodiments, each of the plurality of pores is a microchannel. In some embodiments, the gyroid comprises a 1 mm unit cell. Gyroids are commonly known in the art as structures that lack straight lines and comprise a triply periodic minimal surface. Gyroids are further known to be characterized by a zero mean curvature with local area minimizing. In some embodiments, the gyroids are gyroid infills. Gyroid infills occur when a gyroid pattern is used as an internal geometry of a structure. The gyroids promote filling of the porous layer 12 and create a pressure gradient for vessel sprouts, such as, for example, for anastomosis or angiogenesis into hypoxic regions of the ex vivo tissue support system. The porous layer 12 is configured to increase mass transport approximately 70-fold compared to a typical track-etched membrane, which can increase further during perfusion. In some embodiments, a porosity of the porous layer 12 ranges from 40% to 90%. In some embodiments, the porosity of the porous layer 12 is 45%, as illustrated in FIG. 4B. In some embodiments, the porosity of the porous layer 12 is 60%, as illustrated in FIG. 4C. In some embodiments, at least one of the insert 10 and the porous layer 12 comprises a porosity gradient. FIG. 4D provides a top view of the porous layer 12 with a porosity gradient, according to an aspect of this disclosure. In some embodiments, the porosity gradient comprises a plurality of pores. In some embodiments, the porosity gradient comprises a lower density of pores in the center of the porous layer 12 relative to an outer boundary of the porous layer 12. In some embodiments, the porous layer 12 comprises microneedles. In some embodiments, the porous layer 12 comprises porous microneedles or hollow microneedles.

With reference to FIGS. 4E-4H, in some embodiments, the porous layer 12 comprises microneedles as illustrated in FIG. 4E. In some embodiments, the microneedles are connected by a series of microchannels as illustrated in FIG. 4F, which provide hollow microneedles that are in fluid communication with a perfusable microchannel. In some embodiments, the microneedles are hollow as illustrated in FIG. 4G. In some embodiments, the microneedles are solid as illustrated in FIG. 4H. In some embodiments, the porous layer 12 comprises a mesh structure, as illustrated in FIGS. 4I-4J.

### IV. Samples, Cells and Tissues

In various aspects, this disclosure provides insert cartridges and devices for ex vivo skin tissue systems that are adaptable to a variety of tissue and cell types, which may be arranged in ways so as to optimize tissue models for accuracy and reliability of resulting measurements. For example, tissue and cell types may be selected and curated for promoting functionality of the skin, cell-cell interaction, regenerative capacity, immunocompetency, metabolic activity, mechanical properties, and vascularization, among other critical characteristics of model tissue platforms. In some embodiments, the methods described herein comprise contacting a tissue sample onto the first side of the porous layer. In some embodiments, the tissue sample is a re-engineering skin tissue. In some embodiments, the tissue sample is a native skin tissue. In some embodiments, the tissue sample is a synthetic skin tissue. In some embodiments, a source of the tissue sample may be human, non-human primate, or non-human mammals including, without limitation: dog, cat, sheep, horse, pig, rabbit, mouse, rat, goat, llama, duck, chicken, or turkey. In some embodiments, the skin tissue is a biopsy, such as from a biopsy punch. In some embodiments, the skin tissue is reconstructed three-dimensional (3D) skin with single or multiple cell types. In some embodiments, the skin tissue comprises at least one of epidermis, dermis, and hypodermis. In some embodiments, the skin tissue comprises cells and ECM components from vasculature and hypodermis. In some embodiments, the skin tissue comprises one or more of hair follicles, nails, sweat glands, and sebaceous glands. In some embodiments, the skin tissue has a 20 mm diameter. In some embodiments, the skin tissue has a particular skin type, such as, for example, dry, oily, sensitive, and/or a combination thereof. In some embodiments, the skin tissue has a particular skin condition, such as, for example, acne, eczema, psoriasis, dermatitis, rosacea, hyperpigmentation, discoloration, sun damage, and/or a combination thereof. In some embodiments, the skin tissue comes from individuals that vary in one or more of the following: age, ethnicity, and gender. In some embodiments, the skin tissue may vary by body region.

In some embodiments, the methods described herein comprise seeding cells onto at least one of the first side of the porous layer, an internal region of the porous layer, and the second side of the porous layer. In some embodiments, the seeding is performed using bioprinting, which may include, for example, methods of 3D printing. In some embodiments, the seeding is performed using perfusion. In some embodiments, the seeding is performed manually by a user. In some embodiments, the seeded cells are endothelial cells. In some embodiments, the seeded cells are primary cells or immortalized cells. In some embodiments, the seeded cells are induced pluripotent stem cells (iPSCs). In some embodiments, the seeded cells are primary cells from a different donor than that of the tissue sample. In some embodiments, the seeded cells are isolated cells from the same donor as that of the tissue sample.

In some embodiments, the 3D printing is performed using stereolithography. In some embodiments, the bioprinting is performed using digital light processing (DLP) printing. In the case of 3D printing cells are seeded on the structure after printing. In the case of bioprinting, the cells may be incorporated into the porous structure or seeded in after. One difference between the 3D printing and bioprinting is the materials of use. For example, bioprinting allows for the manufacture of the porous structure with gel materials, such as collagen, gelatin, hyaluranic acid, PEG, etc. These materials are likely more biocompatible than the plastics produced with 3D printing.

In some embodiments, the methods described herein comprise depositing an adipose layer onto one or more of the first side of the porous layer, and internal region of the porous layer, and the second side of the porous layer. In some embodiments, the adipose layer comprises one or more of adipose-derived stem cells, adipocytes, and pre-adipocytes. In some embodiments, the adipose layer comprises one or more of fragmented tissue or isolated cells from adipose tissue. In some embodiments, the adipose layer comprises restructured adipose tissue, wherein the restructure adipose tissue is constructed via (1) one or more of bio printing and 3D culturing of adipose-derived cells then (2) merging the adipose layer with a skin tissue layer, wherein the skin tissue comprises epidermis, dermis, and hypodermis. In some embodiments, the adipose layer is obtained from a same donor as the tissue sample. The methods described herein further comprise sealing the adipose layer with a gas-permeable film. In some embodiments, the film is an adhesive film. In some embodiments, the gas-permeable film is medical grade. In some embodiments, the gas-permeable film is a polyurethane adhesive. In some embodiments, the gas-permeable film is a polyurethane acrylic adhesive. In some embodiments, the gas-permeable film is SanidermR. In some embodiments, the adipose layer may be added within the porous layer. In some embodiments, the adipose layer may be added under the porous layer. In such embodiments, the adipose layer may be held in place due to its negative buoyancy. The methods described herein further comprise culturing at least one of the tissue sample and adipose layer. In some embodiments, the culturing is for a time period of at least one week. In some embodiments, the culturing is for a time period of at least two weeks.

In some embodiments, the methods described herein further comprise seeding a cell composition within the porous layer between the first side and the second side. In some embodiments, the seeded cell composition comprises at least one of: endothelial cells, primary cells, iPSCs, keratinocytes, fibroblasts, melanocytes, resident immune cells, circulating immune cells, stem cells, pre-adipocytes and adipocytes. In some embodiments, the resident immune cells comprise at least one of Langerhans cells, dendritic cells, macrophages, mast cells, monocytes, and lymphocytes. In some embodiments, the circulating immune cells comprise at least one of neutrophils. T lymphocytes. B lymphocytes, natural killer cells, monocytes, and dendritic cells. In some embodiments, the stem cells comprise at least one of adipose-derived stem cells, epidermal stem cells, and dermal stem cells. In some embodiments, the stem cells are isolated from a tissue sample. In some embodiments, the stem cells are configured to provide regenerative capabilities to the skin sample. The regenerative capabilities of the stem cells are helpful for further extending lifetime and health of the tissue sample. In some embodiments, the seeded cell composition comprises one or more of 200,000 to about 2,000,000/mL of adipose-derived stem cells and 500,000 to about 10,000,000/mL of dermal endothelial cells. In some embodiments, the cells may be from a resident microbiome retained on the tissue or on a surface of the tissue, such as from a donor skin tissue. In some embodiments, the cells from a resident microbiome may be microbes from a skin microbiota. In some embodiments, probiotics can be used as a single-or co-treatment agent in the ex vivo skin model system. In some embodiments, the microbiome may be sourced depending on the type of cells, a donor age, a region of skin, etc. In some embodiments, the microbes from the skin microbiota may be assessed as a potential therapeutic.

In some embodiments, the culturing comprises perfusing reagents through the porous structure, wherein the reagents comprise at least one of: Williams E medium, Glutamax, non-essential amino acid solution. ITS (insulin, transferrin, selenium. BSA, and linoleic acid), hydrocortisone, Vitamin C, epinephrine, serum, blood substitute, penicillin and streptomycin.

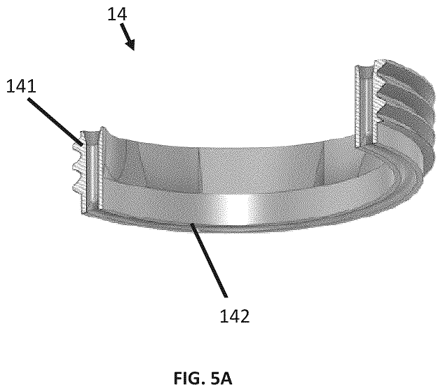

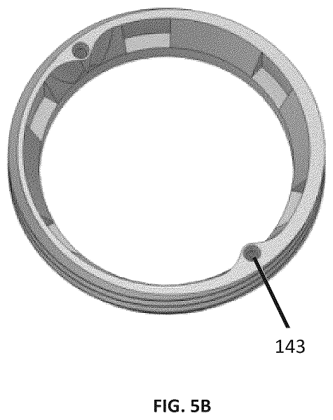

FIGS. 5A-5B provide cross-sectional and perspective views, respectively, of an exemplary embodiment of the compression unit 14. In some embodiments, the compression unit 14 comprises a microchannel 142 that interfaces the tissue, such that a user may seal the compression unit 14 to the tissue. In some embodiments, the compression unit 14 comprises an injection port 143 in fluid communication with the microchannel 142. With this embodiment, a user may tighten compression unit 14 against the skin tissue sample 11, then inject liquid medical-grade adhesive into the port 143, which enters the microchannel 142 and seals an interface between the tissue sample 11 and the compression unit 14, allowing for maintenance of an air-liquid-interface.

### V. High-Content Model Systems

As described above, an ex vivo skin tissue support system may be in the form of alternative embodiments for a variety of different purposes. In some embodiments, for example, the insert 10 is conditioned to form a high-content model (HCM) system. As described further herein, an HCM may comprise an ex vivo tissue model with a complexity and a supportive environment that more closely mimic those experienced by in vivo human tissue from a functional and immunocompetency standpoint.

An HCM may include a full thickness skin biopsy, with the incorporation of engineered vasculature and/or adipose tissue supplied from a same donor of the skin biopsy. An HCM may also include modifications of the surrounding environment to increase longevity and maintain complexity, such as, for example, by providing nutrition that supports growth and maintenance of vasculature. As a result, upon application of one or more test compound or biologic, the HCM may be more accurate in mimicking a tissue response in a living human. Thus, the HCM may also be used to detect a multitude of efficacy claims, complex biological responses, and mechanisms with numerous “high-content” readouts. The embodiments described herein may further be designed for compatibility with commercially available automation and analysis tools. Further, the HCM may function as a long lasting real human skin-based model that may be used as a correlate for product consumer studies.

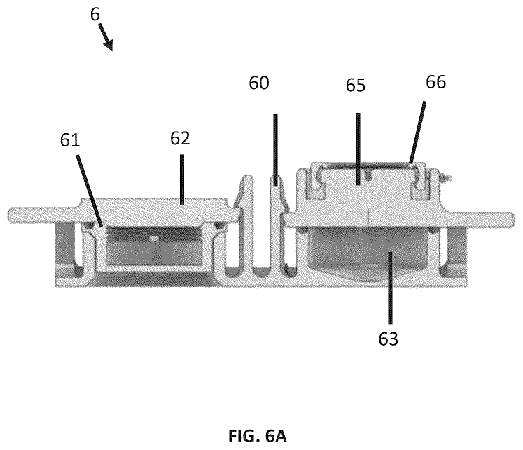

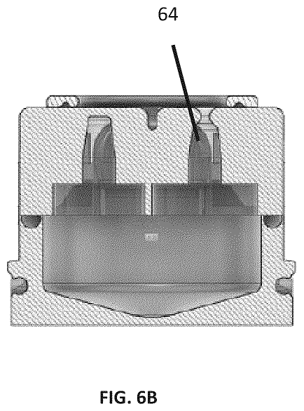

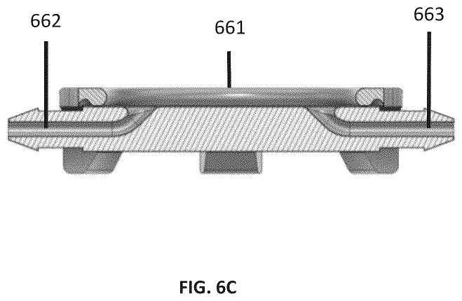

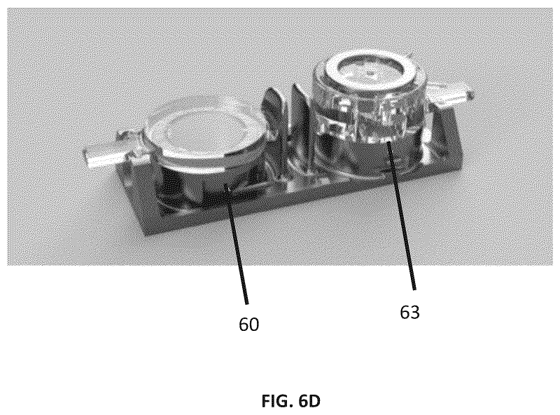

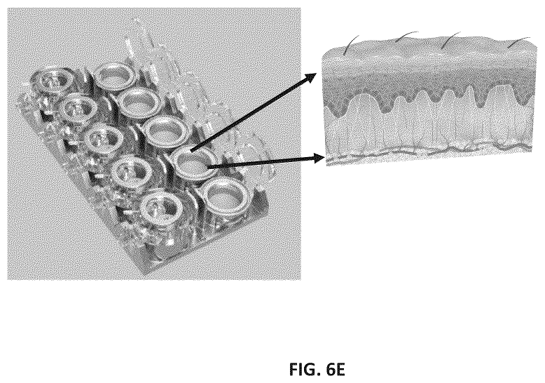

In some embodiments, an HCM described herein may comprise one or more of the following features: (1) a full thickness skin biopsy, (2) the incorporation of engineered vasculature and adipose tissue from the same donor of the skin, (3) modifications of the surrounding environment to increase longevity and maintain complexity, and (4) ability to detect various efficacy claims, complex biological responses, and mechanisms with numerous high-content readouts. In some embodiments, the HCM system comprises a recirculation system. FIGS. 6A-6D illustrate various views of a recirculation system 6 according to several embodiments of this disclosure. The HCM incorporates the ex vivo tissue model into the recirculation system 6 for continuous perfusion by adapting inlet and outlet channels to commercially available automation and analysis tools, including autosamplers. Further, the recirculation system 6 allows for continuous perfusion without the need for multiple reservoirs per samples, or large reservoirs, which may be necessary with single pass flow through the sample. FIG. 6A illustrates a cross-sectional view of a recirculation system 6. In some embodiments, the recirculation system 6 comprises a recirculation cartridge 60, at least one of an insert 61, which may be any one of the inserts described herein, for holding the tissue sample, such as tissue sample 11, an insert cap 62 to seal the insert 61, a media reservoir 63 for allowing media recirculation into the insert 61 and a plurality of check valves 64 (e.g., two check valves), a reservoir cap 65, and a fluid capacitor 66. The fluid capacitor 66 may provide hours of pressure, with a short pressure charge, and eliminates the need for a waste reservoir. The fluid capacitor 66 can be left pressurized with ethanol or water for multiple days without leaks. Air bubbles rise to the top of a membrane 661 and are forced out over time, making it an effective bubble trap as well. FIG. 6B illustrates a detailed cross-sectional view of the reservoir cap 64 comprising integrated check valves 64 to allow for fluid transfer between the media reservoir 63 and the insert 61. FIG. 6C illustrates a detailed cross-sectional view of the fluid capacitor. The fluid capacitor 66 allows one to allow for pressure-based perfusion of the system with a short pulse from a pressure generating device. One advantage is that multiple perfusion circuits can be controlled with a single air pump. When used in combination with two check valves 64, this can allow for recirculation of the fluid only using a single reservoir. In some embodiments, the fluid capacitor 66 comprises at least one of a membrane 661, a fluid inlet 662 and a fluid outlet 663. The fluid capacitor 66 is configured to provide hours of pressure, with a short pressure charge, eliminating need for a waste reservoir. Air bubbles rise to the top of the membrane 661. FIG. 6D provides a perspective view of the recirculation system 6, and FIG. 6E provides a perspective view of a cartridge with a plurality of recirculation systems 6. In some embodiments, the recirculation system 6 is configured to replicate model systems in an HCM. In some embodiments, the HCM incorporates the tissue sample comprising vasculature and adipose tissue from a same donor of the tissue sample. The benefit of the high-content model is increased longevity, maintaining complexity of the tissue sample, and is adapted for use with real human skin samples containing varying skin cells, appendages, and structures. In some embodiments, the recirculation system 6 is adapted to be compatible with commercially available automation and analysis tools.

### VI. High-Throughput Model Systems

In various aspects, this disclosure provides an alternative embodiment to the HCM, for instance, a high-throughput cartridge (HTC) system. Contrary to an HCM, an HTC may comprise an ex vivo tissue model, such that the ex vivo tissue model may comprise a full or partial thickness tissue biopsy while only being used to measure key or select toxicity and efficacy outcomes. The embodiments of the HTC described herein may be compatible with SBS microtiter plate formats, and provide at least 5× more throughput than off-the-shelf biopsy models. Further, low humidity air may be applied through a lid to the HTC, such that a stronger humidity gradient may be produced across the tissue for increased media transport.

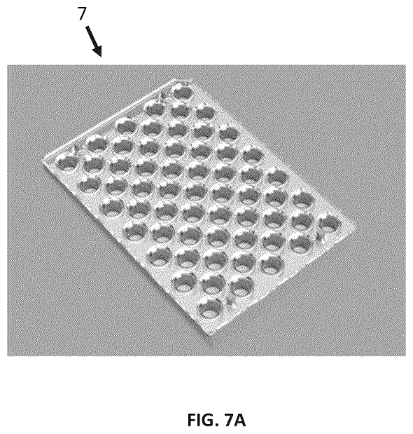

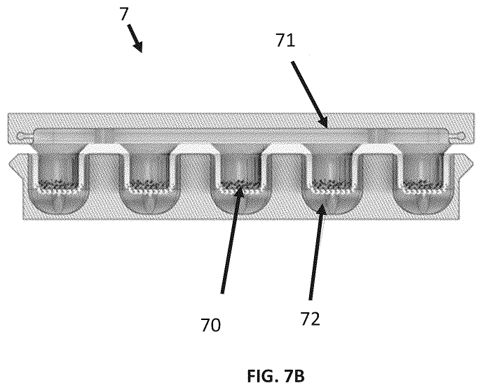

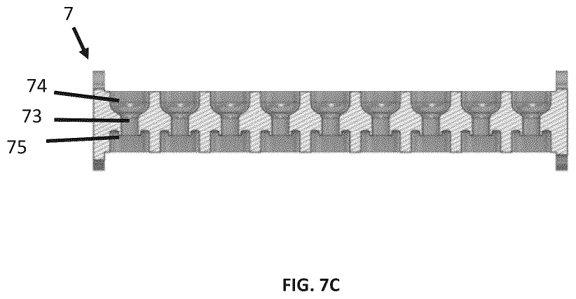

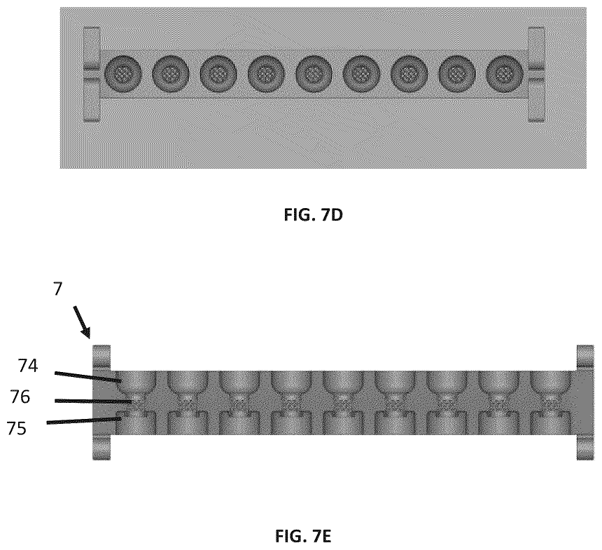

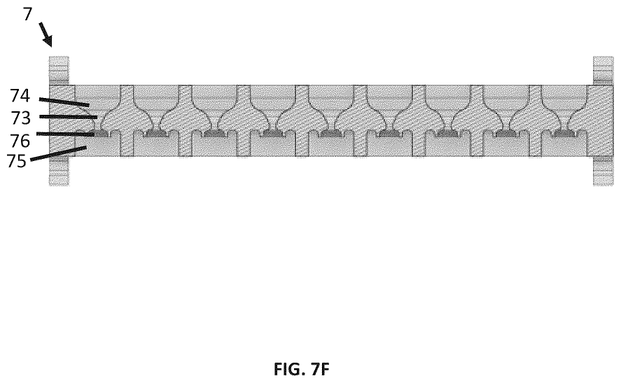

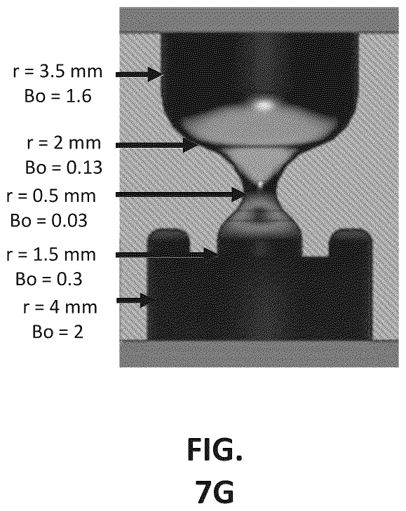

With reference to FIGS. 7A-7B, a perspective and cross-sectional view, respectively, are illustrated of a high-throughput cartridge (HTC) 7. The HTC 7 is generally for use for high-throughput screening of natural and/or synthetic ingredient libraries. In some embodiments, the HTC 7 contains at least fibroblast and keratinocytes. In some embodiments, each of a plurality of inserts 70 is inserted into a slot of a plurality of slots of the HTC 7. In some embodiments, the plurality of slots of the HTC 7 are arranged to be in a 96-well configuration. In some embodiments, the HTC 7 comprises a lid 71. In some embodiments, the lid 71 has fluid channels for perfusing air over the tissue sample. In some embodiments, each insert of the HTC comprises a basal media well 72. The HTC 7 provides the benefit of high-throughput analysis and screening of the tissue sample in a complex system.

The HTC 7 may be set up in a “hanging-drop” style that maintains an air-liquid interface and transports nutrients and waste via a basal interface, as illustrated, in FIGS. 7C-7G. In some embodiments, there is a reservoir on either side of a via 73, e.g., a drop reservoir 74 and a basal reservoir 75, to form a dual-reservoir system, as illustrated in FIG. 7C. By having reservoirs on both sides, and adding a fibroblast 3D culture, keratinocytes are cultured under submerged culture for initial proliferation. The dual-reservoir system further allows for the HTC 7 to be rotated 90 degrees so the fluid in the reservoirs does not exert a significant hydrostatic pressure on a gel droplet in the drop reservoir 74 while the epidermis is maturing. For example, a gel droplet with fibroblasts may be generated and polymerized in the drop reservoir. Keratinocytes may be seeded on the outside of the gel droplet, the system may be cultured in submerged conditions for initial proliferation, the media may be removed from the drop reservoir 74, kept on the basal reservoir 75, and an air liquid interface may be established. In some embodiments, the via 73 has a larger diameter such to increase the surface area between a drop reservoir 74 and a basal reservoir 75. In some embodiments, a mixture of media, gel, and cells is placed in the basal reservoir 75 and the mixture wicks through the via 73 to form the gel droplet on the other side. The pinning geometries of the via 73 and the basal reservoir 75 keep the droplet formed in a stable and consistent geometry. The drop reservoir 74 and basal reservoir 75 are not symmetric, such that the drop reservoir 74 has specific pinning features to form the gel droplet. Once the gel in the gel droplet has been cross linked (via temperature, pH change or UV-crosslinking), a layer of keratinocytes may be seeded on the surface of the gel droplet. In some embodiments, the via 73 comprises a porous layer 76, as illustrated in FIG. 7D-7E. In some embodiments, the via has a smaller diameter to reduce the surface area between a drop reservoir 74 and the basal reservoir 75, as illustrated in FIG. 7F. In some embodiments, the via 73 has a straight inner wall, such to improve imaging capabilities.

FIG. 7G provides a close-up view of a single insert in the HTC system, according to some aspects of this disclosure. FIG. 7G is labeled with example geometries throughout the insert, including diameters of each of the drop reservoir, the via at a proximal end, a central point, and a distal end, and the basal reservoir. The geometries are designed to function regardless of orientation to allow flipping of the system during cell seeding, differentiation and maturation. This is achieved by allowing the Eötvös number (Eo), also known as a Bond number (e.g., Bo)<1, as provided in the equation below:

$$Eo = Bo = \frac{Δρ {gL}^{2}}{γ} \qquad ( 1 )$$

The Bond number provides a ratio between the gravitational forces and surface tension forces in the system. When Bo>1, gravity dominates, and the droplet shape will be dominated by its mass. Δρ is the difference in density of each of the two phases of fluids, g is gravitational acceleration, L is the characteristic length—for example, the radii of curvature for a drop—and γ is the surface tension. When Bo<1, the stability of the droplet will be dominated by the surface tension of fluid and not the orientation of the cartridge.

### VII. Data Analysis Workflow (DAW)

Data obtained from monitoring ex vivo tissue support systems described herein is incorporated for use in a data analysis workflow (“DAW”). In some embodiments, a DAW described herein comprises trained algorithms for predicting outcomes or assay results based on input characteristics such as physiochemical structural, or taxonomical properties of a composition, compound or biologic of interest, also referred herein as a test agent. The test agents may be evaluated using skin tissue samples or skin tissue arrays incorporated within an ex vivo tissue support system described herein, also referred herein as an ex vivo tissue support system. In some aspects, the ex vivo tissue support system comprises: an insert comprising at least one opening(s) and a wall; a sealing unit along an inner wall of the insert; a tissue sample, such as, for example, a skin tissue sample positioned inside the insert via the sealing unit; and a porous layer positioned inside the insert, wherein the porous layer is configured to promote mass transport into and vascularization of the tissue sample. In some aspects, the porous layer has a first side facing the at least one opening(s) and a second side facing opposite the at least one opening(s) of the insert. In some aspects, the ex vivo skin tissue support system is capable of supporting tissue viability for longer than previously reported for such tissue support systems, such as, for example, longer than a week or longer than two weeks. More details about the ex vivo tissue support system are described herein.

In some aspects, described herein are methods for skin tissue analysis, which may include methods for selecting therapeutic agents. In some aspects, the method comprises: (a) contacting a skin tissue array with at least one test agent, (b) measuring a plurality of datasets following contacting the skin tissue array with the test agents, (c) mapping the measured plurality of datasets based on a scoring panel result, and (d) applying a machine learning algorithm to the mapped datasets to generate at least one threshold criteria for selecting the therapeutic agent from the at least one test agent based on the mapped data. In some aspects, this method forms a Extracting-Mapping-Applying workflow for streamlining experimentation of skin tissue samples or skin tissue sample arrays with test agents.

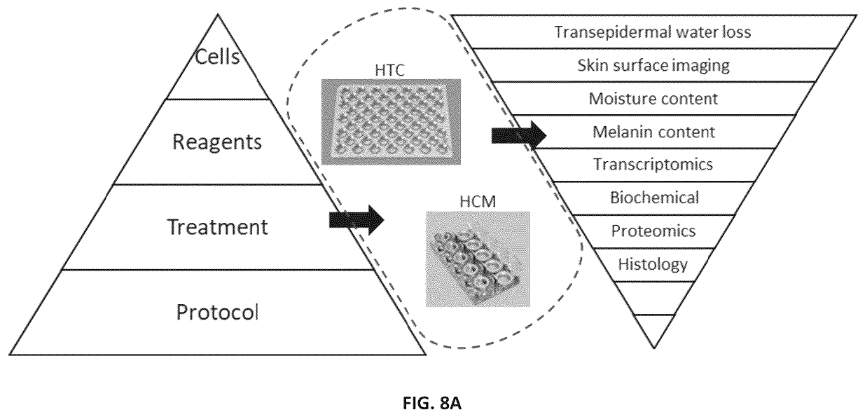

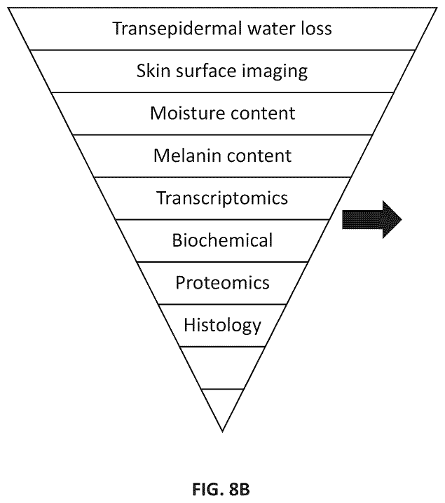

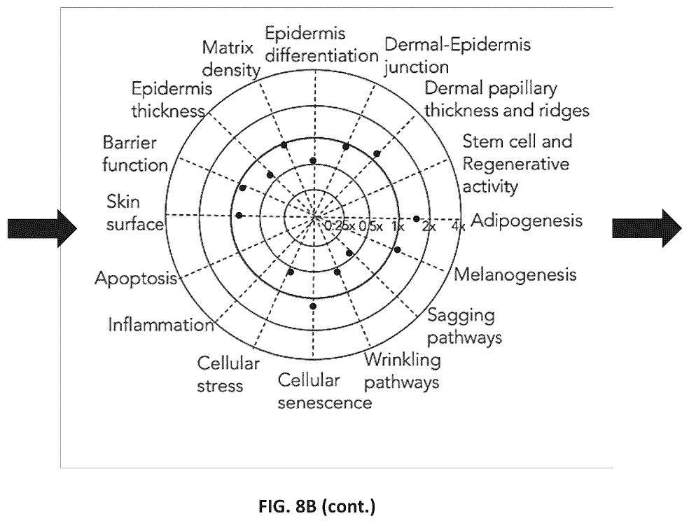

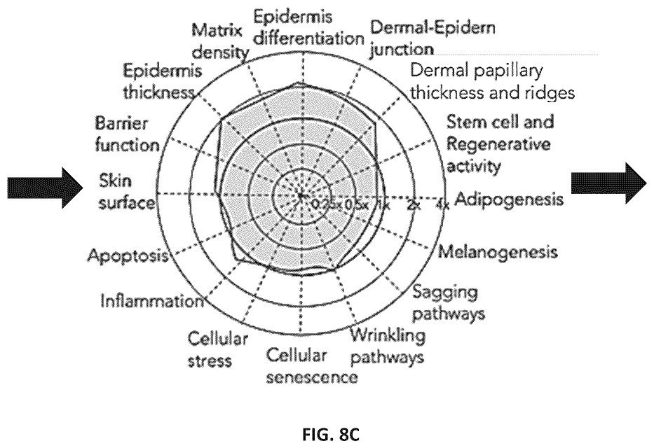

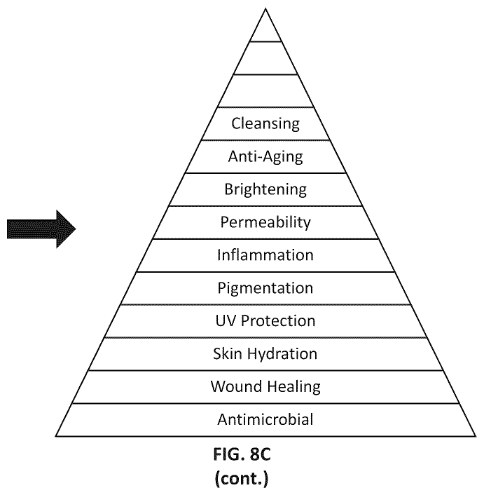

Referring to FIG. 8A, a diagram illustrating a data extraction workflow prior to implementing the Extracting-Mapping-Applying workflow is provided for visualization purposes. In some aspects, the data extraction workflow comprises applying a plurality of inputs to a skin tissue sample in an ex vivo tissue model as obtained or cultured from an ex vivo tissue support system as described herein, such as, for example, one or more of a high content model (HCM) and high throughput cartridge (HTC) model described in further detail below. In some aspects, the plurality of inputs comprise one or more of: the type of protocol used (cell-and/or tissue-dependent), the treatment (e.g., the test agent), reagents used, and the cells and/or tissue used. In some embodiments, non-limiting examples of the substance required for cell metabolism testing include transferrin, human serum albumin, putrescine, ethanolamine, carnitine, linoleic acid and linolenic acid. In some embodiments, non-limiting examples of the reducing agent include vitamin C, sodium selenate, sodium pyruvate and glutathione. In some embodiments, non-limiting examples of antioxidants comprise at least one antioxidant selected from the group consisting of: glutathione (reduced), dithiothreitol (DTT), vitamin E, vitamin K3, vitamin D2 or calciferol, niacin, niacinamide, and ascorbic acid. In some embodiments, non-limiting examples of reagents for lipid synthesis comprises one or more of: cholesterol, linoleic acid, lipoic acid, and o-phosphoryl ethanolamine. In some embodiments, one or more reagents for hormones testing comprise one or more of: progesterone, testosterone, hydrocortisone, and estrogen. In some embodiments, one or more reagents as growth factors comprise one or more of: insulin and epidermal growth factor (EGF). In some embodiments, the reagents may also contain antibiotics if desired, such as penicillin and/or streptomycin. In some embodiments, the reagents used comprise Williams E medium, Glutamax, non-essential amino acid solution, insulin, transferrin, selenous acid, BSA, linoleic acid, hydrocortisone, Vitamin C, serum, blood substitute, penicillin and/or streptomycin. In some embodiments, the reagents used comprises Williams E medium, Glutamax, EGF, transferrin, insulin, progesterone, testosterone, 17B-estrodiol, o-phosphorylethanolamine, selenious acid, linoleic acid, BSA, triiodothyronine, hydrocortisone, cholera toxin, HEPES, and other components.

In some embodiments, the data extracting comprises contacting an ex vivo tissue model with a test composition, compound or biologic. The contacting may be performed by one or more of topical application, subcutaneous application, systemic application, intradermal administration, infusion, perfusion, and injection. After contacting, the data extraction may comprise performing one or more of the in vitro assays to obtain a corresponding clinical measurement, which include but are not limited to one or more of the following: niacinamide, retinol, hyaluronic acid, glycolic acid, and ceramides. In some embodiments, the data comprises measurements from one or more of: a skin surface, histology staining, immunostaining, biochemistry assay, clinical sensors, and gene expression. As described herein, data generated from the in vitro assays are used as inputs for machine learning to iteratively generate optimal algorithms for performing one or more of: substantiating and generating product claims for skin concerns, performing broad efficacy and/or toxicity screening of chemical libraries for quantitative comparison to product benchmarks, and running virtual screens of the therapeutic agent.

In some aspects, the skin tissue array comprises compartments for skin tissue growth comprising an epidermal and/or a dermal tissue layer, a porous layer, and an adipose tissue layer. In some aspects, the porous layer comprises a structure with openings sufficient for vascularization from the adipose tissue layer into the epidermal and/or dermal tissue layer. In some aspects, the skin tissue array is viable for longer than a week. In some embodiments, the skin tissue array is viable for a time period of at least two weeks. In some aspects, the scoring panel result is calculated according to an intensity and effectiveness of each measurement of the measured plurality of datasets, as described above. In some aspects, each of the at least one threshold criteria corresponds to at least one biological pathway.

In some aspects, the Extracting-Mapping-Applying workflow may be used for identifying structure-activity correlations and relationships, for instance, quantitative structure-activity relationships (QSARs), quantitative structure-property relationships (QSPRs), structure affinity relationships (SAFIR), etc., that may be used to significantly accelerate the therapeutic agent selection process. The therapeutic agent selection process disclosed herein implements an intelligent and data-enabled discovery process to optimize the selection of therapeutic agents based on applied test agents. The therapeutic agent selection process may significantly leverage recent advances in high throughput therapeutic agent selection to rapidly search through a diverse parameter space in the Extracting-Mapping-Applying workflow cycles of experimentation for providing agent-specific and robust therapeutic agent characteristics that provide the most stable and predictable behavior. Machine learning may be used to analyze the data to automatically search and identify structure-activity correlations and relationships.

FIGS. 8B-8C provides a diagram describing an example Extracting-Mapping-Applying workflow for data extraction, data mapping, and claim quantification of a test agent according to one or more aspects of the present disclosure. In some aspects of the present disclosure, data extraction can include sources from one or more of histology staining, immunostaining, biochemistry assay, clinical sensors, skin surface analysis, and gene expression. In some aspects of the present disclosure, data mapping can include one or more of epidermis differentiation, matrix density, epidermis thickness, barrier function, skin surface, dermal-epidermis junction, dermal papillary thickness and ridges, stem cell and regenerative activity, adipogenesis, melanogenesis, sagging pathways, wrinkling pathways, cellular senescence, cellular stress, inflammation, and apoptosis. In some aspects of the present disclosure, claim quantification can include analyses of one or more of anti-aging, hydration, toxicity, and irritation.

In some embodiments, the Extracting-Mapping-Applying workflow may be validated for representative test agents, such as one or more of the following: niacinamide, retinol, hyaluronic acid, glycolic acid, and ceramides. For each test agent, a structure-activity correlation and relationship machine learning model may be developed and may be validated with each new dataset. These models may be used in subsequent new designs to create an iterative workflow (e.g., Extracting-Mapping-Applying workflow of FIGS. 8B-8C) once the accuracy of each test-agent-specific model reaches or exceeds a threshold accuracy.

In some aspects, the methods of testing compositions comprises selecting therapeutic agents. In some aspects the methods of testing compositions use big data and diverse datasets generated, for example, from the ex vivo tissue support systems, and processed using machine learning algorithms as described herein to substantiate and generate evidence-based skincare product claims for skin concerns, perform broad efficacy and/or toxicity screening of chemical libraries for quantitative comparison to product benchmarks, and train artificial intelligence (AI) models to run virtual screens. In some embodiments, methods of structural prediction as described herein can employ machine learning and computational intelligence techniques, such as deep neural networks, and combinations of supervised, semi-supervised and unsupervised learning techniques. In some embodiments, methods of therapeutic selection as described herein employ a supervised algorithm (by way of non-limiting example. linear region, random forest classification, decision tree learning, ensemble learning, bootstrap aggregating, and the like). In some embodiments, methods of therapeutic selection as described herein employ a non-supervised algorithm (by way of non-limiting example, clustering or association). Iteration using machine learning using the methods described herein allows for robust simulation beyond the capability of singular data modes alone, which allows for far more predictive (and biologically relevant) algorithms that produce valuable therapeutic agent selections.

The methods and systems described herein may leverage supervised machine learning models, for example, to develop therapeutic agent selection criteria by elucidating the structure-activity correlations and relationships. This may be accomplished by iteratively applying the Extracting-Mapping-Applying workflow by implementing a robust and intelligent high throughput process as described hereinbelow. This workflow may utilize a diverse range of test agent characteristics with a machine learning model to rank variable dependencies so as to reveal structure-function relationships that may otherwise be difficult to determine using hit or miss-type rational designs alone.

In some embodiments, the datasets used to train the machine learning algorithm comprise one or more of: (a) databases filled with datasets produced from testing with one or more of the HTC and HCM systems, and (b) existing public databases such as ChemSkin, SkinSensDB, PubChem, ClinicalTrial.gov, ChEMBL, OpenFDA, DrugBank, Binding DB, NCBI GEO, GO, Human Cell Atlas, and COCONUT. The datasets may be sources from in vivo or in vitro data, such as, for example, from artificial skin system(s), in vivo skin tissue(s), in vitro skin tissue model(s), lab grown full thickness skin system(s), and/or other ex vivo skin model(s), depending on the test agent. In some aspects, model data from such databases may be continuously validated against the mapping of the test agent's Extracting-Mapping-Applying workflow to extract structure-activity correlations and relationships.

In some embodiments, measurements generated from the skin surface comprise measurements from one or more of: dermoscopy, mexameter, cutometer, and photography. In some embodiments, measurements generated from the histology staining comprise measurements from one or more of: hematoxylin & eosin (H&E), Masson's trichrome, and Movat pentachrome.

In some embodiments, measurements generated from the gene expression comprise measurements from one or more of: dermal differentiation, dermal-epidermal junction, dermal marker, matrix metalloproteinases (MMPS), MMP inhibitors, regeneration, wrinkling, sagging, inflammation, intrinsic apoptosis, extrinsic apoptosis, anti-apoptosis, immune cell markers, skin elasticity, skin rejuvenation, and antioxidant defense. In some embodiments, measurements generated from the clinical sensors comprise measurements from one or more of: moisture, oil, pH, dermoscopy, photography, melanin content, and ultrasound.

The clinical measurements obtained during the data extracting may then be pooled together into a database for one or more of algorithm training and/or mapping. In some embodiments, the database may be structured in away such that the data may be fed into a training set for a machine learning algorithm with minimal user manipulation. In some embodiments, the mapping comprises capturing one or more characteristics such as, for example: epidermal differentiation, epidermis thickness, dermal-epidermal junction, barrier function, cellular stress, cellular senescence, dermal marker, MMPs, MMP inhibitors, regeneration, wrinkles, sagging, inflammation, intrinsic apoptosis, extrinsic apoptosis, anti-apoptotic, matrix density, dermal papillary thickness and ridges, stem cell and regenerative activity, adipogenesis, melanogenesis, immune cell markers, skin elasticity, skin rejuvenation, and antioxidant defense.

The DAW may use one or more of the following as input data obtained from one or more of the HCM and the HTC, sets for improved predictive algorithms compared to others using singular data modes; skin surface imaging, histology staining, immunostaining, biochemistry assay, transcriptomics, proteomics, biochemical data, and clinical sensor data.

In some embodiments, the monitoring comprises: (a) contacting an ex vivo tissue model with a test composition, compound or biologic; (b) generating a gene or protein expression/pathway map for the ex vivo tissue model, wherein the gene or protein expression/pathway map comprises data related to the transcription of one or more genes; (c) comparing the gene or protein expression/pathway map for the ex vivo tissue model to a control gene or protein expression/pathway map; and (d) identifying the test composition, compound or biologic as effective for providing a characteristic, benefit or function to the ex vivo tissue model when the gene or protein expression/pathway map for the ex vivo tissue model and the control gene or protein expression/pathway map are in agreement. Each of the gene or protein expression/pathway maps may be obtained through one or more of bulk RNA-seq and microarrays. Each of the gene or protein expression/pathway maps may comprise expression datasets from one or more genes or proteins selected from: TJP1, OCLN, KRT (1, 2, 5, 9, 10, 14), EGFR, TGM (1, 3, 5), DSP, LORICRIN, FLG, CASP14, CLDNI, IVL, ITGB (1, 4, 5), LAMA (4, 5), LAMC1, COL4A1, COLSA1, COLIA1, COL3A1, COLI7A1, COL7A1, COL9A1, VCAN, VIM, COL12A1, FBN1, ACTA2, THY1, IN1, FAP, HAS2, TGFB1, HNFIA-AS1, SMIM6, HPSE, EMILIN1, MMP (1, 2, 3, 7, 9-17, 19-21, 23B, 24-26, 28), TIMP (1-4), FGF (2, 7, 9), LMN81, MK167, KRT (15, 19), TP63, SOK2, MNAT1, PSMA1, PSMD2, PSMC2, XPC, PSMB (3, 5), DOB1, PMM2, POLE4, GMDS, CYBRK1, VEGFA, CMAS, RAD23B, RPTOR, TSC1, DDB1, CAB39, RPS6KA2, PIK3R1, PIK3CA, GFPT2, ERCCB, TNF, IL35G, IL12A, ILIB, S100A7, IL33, CXCLB, IL6, ATM, DFFB, MDM2, TPS3, BAD, BAX, DBC3, AJFM1, CHEK2, PMASP1, BID, CASP (3, 7-9), BCL2, BMF, TNFSF10, TRADD, TNFRSF1A, FADD, BCL2, BCL2A1, BCL2L1, MCL1, BCL2L10, BCL2L2. Each of the gene or protein pathway maps may comprise pathway datasets from one or more of epidermis development, peptide cross-linking, retinoic acid metabolism, keratinocyte differentiation, flavonoid glucuronidation, xenobiotic glucuronidation, and epidermis cell differentiation.

In some embodiments, the one or more selection criteria being either positively correlated or negatively correlated with a condition, the selection criteria comprises at least one threshold for quantifying the data. In some embodiments, the condition comprises one or more of: anti-aging, skin hydration, toxicity, brightening, discoloration, pigmentation, UV protection, cleansing, permeability, inflammation, anti-inflammation, antimicrobial, wound healing, skin barrier, epidermal thickness, dermal matrix, cellular stress, cellular senescence, sagging, and wrinkling.

In some embodiments, the input data for skin surface imaging comprises scoring results when comparing images of untreated skin surface to a treated skin surface, wherein the treated skin surface is treated with a test composition as described herein. In some embodiments, the transcriptomics data comprises gene expression data for one or more of the characteristics described herein. In some embodiments, the input data comprises pathology scoring for one or more of the following conditions: parakeratosis, hyperkeratosis, acanthosis, dyskeratotic, keratinocytes, spongiosis, ballooning, edema, and vesicles. In some embodiments, the input data may be from cells related to a microbiome, which may be useful for correlating skin data with test agents used for one or more of maintaining, restoring, and activating microbiota.

In some embodiments, the measurements of the test agents may be mapped as illustrated in FIGS. 8B-8C from any of the databases. These measurements may be compared to the mappings for each characteristic from all machine learning models to establish structure-activity correlations and relationships. The mappings produced may be highly quantitative and systematic, capturing different intensity and efficacy using the scoring systems described herein, thereby offering potential insight to a plurality of functionalities. Finally, the best top performing test agents may be further characterized by one or more of the conditions above.

### VIII. Data Mapping and Analysis

In various aspects, this disclosure provides methods of mapping data from the ex vivo tissue models to extract particular features and/or characteristics-when contacted with at least one test compound or biologic-using a variety of standard techniques and assay panels, and analyzing resulting readouts, such as through scoring, for reliable comparison and/or ranking of test compounds and biologics. The methods described herein may be incorporated for providing validation of the ex vivo tissue models with known active ingredients used in skincare testing (e.g., cosmetic(s), natural ingredient(s), haircare, plant extract(s)/oil(s), sun/light protection, fruit acid(s), vitamin(s)/protein(s), etc.), thus setting a foundation for one or more of: selecting the most sensitive measurement readout(s) and normalizing measurement scales, assessing toxicity and selecting safe therapeutic windows for active ingredients, and benchmarking data to compare novel ingredients.

In some embodiments, the methods described herein comprise monitoring at least one of cell characteristics and tissue characteristics comprising at least one of efficacy, toxicity. pharmacodynamics, and longevity of the tissue sample for contributing to the DAW. The cell and tissue characteristics provide the benefit of mass data generation for downstream analysis rather than relying merely on existing databases. In some embodiments, the monitoring is performed in response to a test composition, compound or biologic applied to an ex vivo tissue model within the ex vivo tissue support system. In some embodiments, the ex vivo tissue support system comprises the HCM or the HTC system, described in further detail below. The ex vivo tissue model may be at any stage of development or growth within the ex vivo tissue support system.

In some embodiments, the monitoring at least one of cell characteristics and tissue characteristics comprises monitoring at least one of: skin structure, anti-aging, skin hydration, brightening, discoloration, pigmentation. UV protection, cleansing, permeability, inflammation, anti-inflammation, antimicrobial, wound healing, skin barrier, epidermal thickness, dermal matrix, cellular stress, cellular senescence, sagging, wrinkling, sensitization, irritation, corrosion, and phototoxicity. TABLE 1 provides a summary of example characteristics. In some embodiments, at least one of the cell characteristics and tissue characteristics are measured when stimulated with active ingredients. TABLE 2 provides example active ingredients used for benchmarking results from the ex vivo tissue support systems described herein. In some embodiments, the monitoring comprises using at least one of using staining and imaging, gene expression, sensing, dermoscopy, and ultrasound. In some embodiments, the monitoring comprises ex vivo assays, wherein the ex vivo assays comprise at least one of hematoxylin and eosin (H&E), other histology and immunostaining (such as, for example, immunofluorescence (IF)), biochemical and immunoassays, and gene expression analysis. In some embodiments, the monitoring comprises measuring at least one of: dermal markers such as Fibrillin, Vimentin, collagen I, collagen III and elastin; dermal-epidermal junction markers such as Integrin B4, Laminin 5, and collagen VII; epidermal differentiation markers such as filaggrin, trichrome, involucrin, transglutaminase, and cytokeratins; lipid markers such as sebaceous lipids, epidermal lipids such as ceramides, cerebrosides and phospholipids; epidermal extracellular matrix components such as glycosaminoglycans (GAGs) and hyaluronic acid, proteoglycans, ECM receptors, and proteases; markers of epidermal cohesion and intercellular cell junctions such as occluding junctions and attachment proteins, such as claudin, occludin, desmogleins; dermoepidermal junction such as integrin V, collagen IV, collagen VII; gap junctions such as connexins; and molecular channels such as aquaporins. In some embodiments, the monitoring comprises measuring at least one of Pro Colla, matrix metalloproteinases (MMPs), serine proteases, reactive oxygen species (H2DCFDA), LC3, AGEs; DNA damage, miR-23a-3p (for hyaluronan synthase 2), miR126 (endothelial); free radical production, and advanced glycation end (AGE). In some embodiments, the monitoring comprises measuring Melanocyte location, Ki 67, gp-100, Fontana-Masson, loricrin, keratin-1, keratin-14, melanogenesis markers (TYR, TRP-1, Pmel17, MITF, etc.); Tyrosinase activity and expression; cyclobutanepyrimidine dimers (CPDs), active caspase 3; fluorescence and LCMS for permeability; cytokines (IL-17, 2, 4, 6, 12, 8, 10, TNF-α, IFN-γ, TGF-β); staining for Keratin 14, Vimentin, Involucrin, and Filaggrin. TABLE 1 provides information on example characteristics that are measured and the endpoints and assays used to measure the characteristics. TABLE 2 provides example active ingredients used for testing with the ex vivo model.

TABLE 1

| Characteristic | Endpoint | In vitro Assay | Clinical Measurement |
|---|---|---|---|
| Skin Structure | Staining & | H&E, IF staining, qPCR | Ultrasound, TEWL |
| | Imaging, Gene | Dermal markers: Fibrillin, Vimentin, | |
| | expression, | collagen I, collagen III and elastin; Dermal- | |
| | Ultrasound | epidermal junction markers: Integrin B4, | |
| | | Laminin 5, Collagen VII; Epidermal | |
| | | differentiation: filaggrin, involucrin, | |
| | | transglutaminase, cytokeratins; Lipid: | |
| | | sebaceous lipids, epidermal lipids such as | |
| | | ceramides, cerebrosides and phospholipids | |
| | | Epidermal extracellular matrix components: | |
| | | glycosaminoglycans and hyaluronic acid, | |
| | | proteoglycans and ECM receptors, | |
| | | proteases; Markers of epidermal cohesion | |
| | | and intercellular cell junctions: occluding | |
| | | junctions and attachment proteins, such as | |
| | | claudin, occludin, desmogleins; | |
| | | dermoepidermal junction, such as integrin | |
| | | V, collagen IV, collagen VII; gap junctions | |
| | | such as connexins; and molecular channels | |
| | | such as aquaporins. | |
| Skin hydration | Sensor | | Corneometer, TEWL |
| Anti-aging | Staining & | H&E, Pro Col1a, Collagen VII, MMPs, | Ultrasound, |
| | Imaging, Gene | serine proteases; Deposition of collagen, | Viscoelasticity and |
| | expression, | elastin, hyaluronic acid (mRNA, protein); | softness sensors, skin |
| | Sensor, | Glycosaminoglycans (GAGs) Reactive | photo |
| | Dermoscopy | oxygen species (H2DCFDA), LC3, AGEs | |
| | | DNA damage, miR-23a-3p (for hyaluronan | |
| | | synthase 2), miR126 (endo); Free radical | |
| | | production, advanced glycation end (AGE) | |
| Brightening/ | Sensor, | Melanocyte location, Ki 67, gp-100, | Melanin sensor, Color |
| Pigmentation/ | Staining & | Fontana-Masson, loricrin, keratin-1, | sensor |
| Discoloration | Imaging, | keratin-14, melanogenesis markers (TYR, | |
| | Dermoscopy | TRP-1, Pmel17, MITF, etc.); Tyrosinase | |
| | | activity and expression | |
| UV irradiation/ | Sensor, | Cyclobutanepyrimidine dimers (CPDs), | Skin photo, mexameter, |
| protection | Imaging, | active caspase 3 | skin temperature, |
| | Assays | | TEWL, pH, ultrasound |
| Permeability | Sensor | Fluorescence quantification, LCMS | TEWL |
| Inflammation | Assays, | Cytokines (IL-17, 2, 4, 6, 12, 8, 10, TNF-α, | Skin photo (rash, etc.) |
| | Imaging | IFN-γ, TGF-β) | |
| Wound healing | Staining & | H&E, Staining for Keratin 14, Vimentin, | Wound photo, size |
| | Imaging | Involucrin, Filaggrin | measurement |
| Irritation | Assays | MTT | |
| Corrosion | Assays | MTT | |
| Sensitization | Assays | MTT, IL-18 | |
| Genotoxicity | | TUNEL | |

TABLE 2

| Ingredient | Origin | Function | Claimed Benefit |
|---|---|---|---|
| Niacinamide | Vitamin B3 | Active Ingredient | Hydration via improved barrier; anti- |
| | | | inflammatory |
| Retinol | Sensor | Active Ingredient | Reduce wrinkles by increasing collagen |
| | | | production and increasing cell turnover; anti- |
| | | | oxidant |
| Hyaluronic | Natural | Active Ingredient | Moisturization by binding to water |
| Acid | component of | Moisturizer | |
| | skin | | |
| Glycolic Acid | AHA (derived | Active ingredient | Reduces hyperpigmentation by increasing cell |
| | from sugar cane) | Moisturizer | turnover |
| Ceramides | Natural | Moisturizer | Restores skin barrier |
| | component of | | |
| | skin | | |

In some embodiments, the input characteristics are scored or classified. In some embodiments, the scoring or classification is performed based on at least one of (1) a percent change in the respective characteristic from a normal condition, and (2) the method used to obtain the characteristic. In some embodiments, percent change score ranges from 0-4, wherein a score of 0 indicates no change, 1 is given when the change is <25%, 2 when the change is 26-50%, 3 when the change is 51-75%, and 4 when the change is 76-100%. In some embodiments, a combination of method classifications are applied to the characteristic. In some embodiments, tool and endpoint inputs are incorporated into the trained algorithms. Such tool and endpoint inputs comprise one or more of: ultrasound, H&E, dermoscopy, photography, melanin content, corneometer, cutometer, mexameter, TEWL, PH meter, thermometer, oil sensor, Periodic acid-Schiff (PAS), trichrome, elastin, collagen IV, collagen VII, pro-collagen I, laminin 5, hyaluronan synthases (HAS), vimentin, filaggrin, keratin 10, keratin 14 (basal), lipid (adipocytes), Ki67, keratin 15, CD34, TUNEL, p53, CPD (via UV damage), ROS, MMPs, L-2, IL-4, IL-6, IL-8, IL-10, IL-17, IL-18, IL-22, IL-27, TNF-a, IFN-g, TGF-b, MTT, RNA seq, and LC-MS. In some embodiments, the tool and endpoint inputs are multiplied by a factor.

In some embodiments, ranking may be performing by one or more of grades and numbers. TABLE 3 provides an exemplary system for a ranking system using grades to quantify effectiveness for addressing a specific endpoint.

TABLE 3
Example Ranking System

| Grade | Anti-Aging | Hydration | Sun Protection |
|---|---|---|---|
| 0 | 0-39 | 0-19 | 0-19 |
| Slightly | 40-79 | 20-39 | 20-39 |
| Mild | 80-119 | 40-59 | 40-59 |
| Effective | 120-159 | 60-79 | 60-79 |
| Very Effective | 160+ | 80+ | 80+ |

The skin care compositions may be generally prepared by conventional methods such as known in the art of making compositions and topical compositions. Such methods typically involve mixing of ingredients in or more steps to a relatively uniform state, with or without heating, cooling, application of vacuum, and the like. The compositions are preferably prepared such as to optimize stability (physical stability, chemical stability, photostability, etc.) and/or delivery of active materials.

The compositions may be in various product forms that include, but are not limited to, solutions, suspensions, lotions, creams, gels, toners, sticks, pencil, sprays, aerosols, ointments, cleansing liquid washes and solid bars, shampoos and hair conditioners, pastes, foams, powders, mousses, shaving creams, wipes, strips, patches, electrically-powered patches, wound dressing and adhesive bandages, hydrogels, film-forming products, facial and skin masks (with and without insoluble sheet), make-up such as foundations, eye liners, and eye shadows, viral and non-viral gene delivery agents, microneedle patches, and the like.

### IX. System and Computer Implementation

In various aspects, this disclosure provides a method for skin tissue analysis, which relies on handling a database associated therewith for compiling and organizing any evaluation or test data obtained from an ex vivo tissue support system described herein. Due to the scalability of ex vivo tissue support system for automation and therefore high-throughput data, it is highly desirable to have a streamlined workflow of data obtained in the laboratory setting to a populated and organized screening library for user access and understanding, whether the purpose is for training intelligent data analytics or clinical trials.

In some aspects, the method for skin tissue analysis comprises receiving a plurality of datasets, the datasets being measured following contacting a skin tissue array with a plurality of test agents; measuring a plurality of datasets following contacting the skin tissue array with the test agents; mapping the measured plurality of datasets based on a scoring panel result, wherein the scoring panel result is calculated according to an intensity and effectiveness of each measurement of the measured plurality of datasets; and applying a machine learning algorithm to the mapped datasets to generate at least one threshold criteria for selecting the therapeutic agent from the at least one test agent based on the mapped data, wherein each of the at least one threshold criteria corresponds to at least one biological pathway. Example threshold are discussed more in Example 7 below.

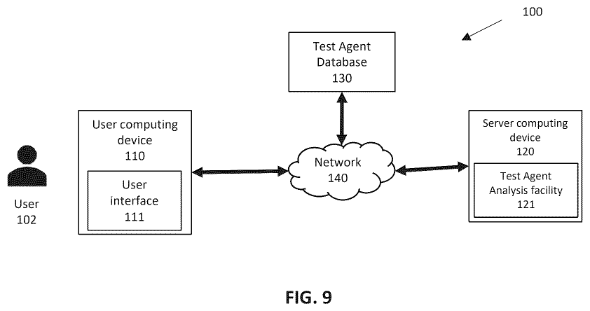

FIG. 9 illustrates a block diagram of a system 100 with which some embodiments may operate for receiving the plurality of datasets. The system 100 can analyze datasets for a plurality of test agents for selection of a therapeutic agent. The system 100 can include a user computing device 110, which may be a desktop or laptop personal computer, smart mobile phone, server, or other suitable device. The user computing device 110 may include a user interface 111 by which the user 102 may interact with the user computing device 110. For example, the user 102 can use the user interface 111 to interface with the test agent database 130 or test agent analysis facility 121 of the server computing device 120. For example, the user 102 may operate the user interface 111 to initiate analysis of a dataset from the test agent database 130 and display analysis results such as calculating a scoring panel result based on an intensity and effectiveness of each measurement in the datasets or generating at least one threshold criteria for selecting the therapeutic agent in the interface 111. The user 102 may additionally or alternatively operate the user interface 111 to input datasets obtained from the test agent database 130, such as output to the user 102 in another interface. Those values may be provided to the test agent analysis facility 121. As a further example, the user 102 may operate the user interface 111 to initiate analysis of the test agent by the test agent database 130 and provision of analysis results (e.g., scoring panel results or threshold criteria) from the test agent database 130 to the test agent analysis facility 121. Results of analysis of the results (received from the test agent database 130 or from the interface 111) by the test agent analysis facility 121 may be output to the user interface 111, such as by being received at the user interface 111 and displayed on the device 110. In some embodiments, as mentioned above, the user interface 111 may include a web interface, such as one or more web pages into which values may be output and which may display results of the analysis by the test agent analysis facility 121, but embodiments are not so limited. The user interface 111 may accept input in a variety of different formats, such as through speech recognition, text input, or other means, as embodiments are not limited in this respect.

The system 100 can include a server computing device 120, which may include a test agent analysis facility 121 configured to analyze factors (e.g., derived from the datasets, such as by the test agent database 130) for the user 102 to rank or select test agents. In some embodiments, the test agent analysis facility 121 may receive information on the factors from the test agent database 130 and/or from the user interface 111. In some embodiments, the test agent analysis facility 121 may output selected test agents that satisfy predetermined criteria as therapeutic agents.

The system 100 can include a network 140 to facilitate communications among the test agent database 130, the user computing device 110, and the server computing device 120. The network 140 can be or include any one or more wired and/or wireless, local-and/or wide-area network, including one or more enterprise networks and/or the Internet.

While the illustration of FIG. 9 includes the user interface on a device 110 separate from the sample analyzer 112, it should be appreciated that embodiments are not so limited. In other embodiments, the user interface 111 may be an interface of the test agent database 130 and may be operated by the user 102. Additionally or alternatively, while the test agent analysis facility 121 is illustrated on a different computing device from the user computing device 110 and the test agent database 130, embodiments are not so limited. In other embodiments, the analysis facility may be implemented on the client computing device or the test agent database 130. In some embodiments, the user interface 111 may not be separate from the test agent analysis facility 121, but instead may be implemented as a single program or software application. In some embodiments, a test agent database 130 may include the user interface 111 and the test agent analysis facility 121, and the interface 111 and facility 116 may be implemented within the same program or application executed on the test agent database 130.

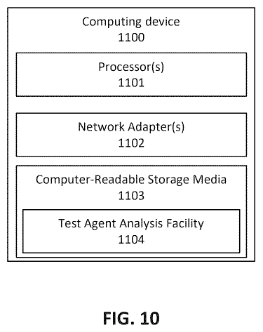

Computer-executable instructions implementing the techniques described herein (when implemented as one or more functional facilities or in any other manner) may, in some embodiments, be encoded on one or more computer-readable media to provide functionality to the media. Computer-readable media include magnetic media such as a hard disk drive, optical media such as a Compact Disk (CD) or a Digital Versatile Disk (DVD), a persistent or non-persistent solid-state memory (e.g., Flash memory. Magnetic RAM, etc.), or any other suitable storage media. Such a computer-readable medium may be implemented in any suitable manner. including as computer-readable storage media 1103 of FIG. 10 described below (i.e., as a portion of a computing device 1100) or as a stand-alone, separate storage medium. As used herein. “computer-readable media” (also called “computer-readable storage media”) refers to tangible storage media. Tangible storage media are non-transitory and have at least one physical, structural component. In a “computer-readable medium,” as used herein, at least one physical, structural component has at least one physical property that may be altered in some way during a process of creating the medium with embedded information, a process of recording information thereon, or any other process of encoding the medium with information. For example, a magnetization state of a portion of a physical structure of a computer-readable medium may be altered during a recording process.

In some, but not all, implementations in which the techniques may be embodied as computer-executable instructions, these instructions may be executed on one or more suitable computing device(s) operating in any suitable computer system, including the exemplary computer system of FIG. 9, or one or more computing devices (or one or more processors of one or more computing devices) may be programmed to execute the computer-executable instructions. A computing device or processor may be programmed to execute instructions when the instructions are stored in a manner accessible to the computing device or processor, such as in a data store (e.g., an on-chip cache or instruction register, a computer-readable storage medium accessible via a bus, a computer-readable storage medium accessible via one or more networks and accessible by the device/processor, etc.). Functional facilities comprising these computer-executable instructions may be integrated with and direct the operation of a single multi-purpose programmable digital computing device, a coordinated system of two or more multi-purpose computing device sharing processing power and jointly carrying out the techniques described herein, a single computing device or coordinated system of computing devices (co-located or geographically distributed) dedicated to executing the techniques described herein, one or more Field-Programmable Gate Arrays (FPGAs) for carrying out the techniques described herein, or any other suitable system.

FIG. 10 illustrates one exemplary implementation of a computing device in the form of a computing device 1100 that may be used in a system implementing techniques described herein, although others are possible. It should be appreciated that FIG. 10 is intended neither to be a depiction of necessary components for a computing device to execute an analysis facility in accordance with the principles described herein, nor a comprehensive depiction.

Computing device 1100 may comprise at least one processor 1101, a network adapter 1102, and computer-readable storage media 1103. Computing device 1100 may be, for example, a desktop or laptop personal computer, a personal digital assistant (PDA), a smart mobile phone, a server, a wireless access point or other networking element, or any other suitable computing device. Network adapter 1102 may be any suitable hardware and/or software to allow for the computing device 1100 to communicate wired and/or wirelessly with any other suitable computing device over any suitable computing network. The computing network may include wireless access points, switches, routers, gateways, and/or other networking equipment as well as any suitable wired and/or wireless communication medium or media for exchanging data between two or more computers, including the Internet. Computer-readable media 1103 may be adapted to store data to be processed and/or instructions to be executed by processor 1101. Processor 1101 allows for processing of data and execution of instructions. The data and instructions may be stored on the computer-readable storage media 1103.

The data and instructions stored on computer-readable storage media 1103 may comprise computer-executable instructions implementing techniques which operate according to the principles described herein. In the example of FIG. 10, computer-readable storage media 1103 stores computer-executable instructions implementing various facilities and storing various information as described above. Computer-readable storage media 1103 may store analysis facility 1104.

While not illustrated in FIG. 10, a computing device may additionally have one or more components and peripherals, including input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computing device may receive input information through speech recognition or in other audible format.

### EXAMPLES

### Example 1

### Preparation of an Ex Vivo Tissue Model

The purpose of this example is to incorporate adipose stem cells (ASC) into an insert.

Step 1. Prepare Fibricol and 10× minimum essential medium (MEM) on ice. Chill serological pipette and pipette tips. Bring NaOH solution in biological safety cabinet (BSC).

Step 2. Prep ASC cells: thaw and resuspend in Adipose nutrition media, spin down at 200 g 5 min.

Step 3. Prepare gel solution (Ratio=Fibricol (8): 10× MEM (1): ASC suspension (1))

Step 3a. Mix fibricol (4 mL) and 10× MEM (500 uL) using cold pipettes/tips in cold 15ml conical tube on ice.

Step 3b. Add 2 uL of 5N NaOH, mix well, and monitor medium color. Repeat this step until see color change to pH around 7.2˜7.4. (Total 5 uL of NaOH added)

Step 4. After centrifugation, aspirate supernatant, and resuspend the cell pellet (total 500,000 cells) in 1 mL of Adipocyte nutrition medium.

Step 5. Add 500 uL of cell suspension in the gel solution on ice and mix well.

Step 6. Flip the insert (first side down) in 6 well plate (mesh/porous part on top), add 500 uL of gel+cell solution gently on the center. Monitor if the solution spreads, spread the solution using the pipette tip on the surface of insert.

Step 7. Incubate the well plate in the CO2 incubator for 40 min

Step 8. Flip back the insert (first side up) in 6 well plate and add 3 mL of Mixture media.

Step 9. After 3 hours, place the tissue in the insert.

### Example 2

### Histology Analysis of Tissue

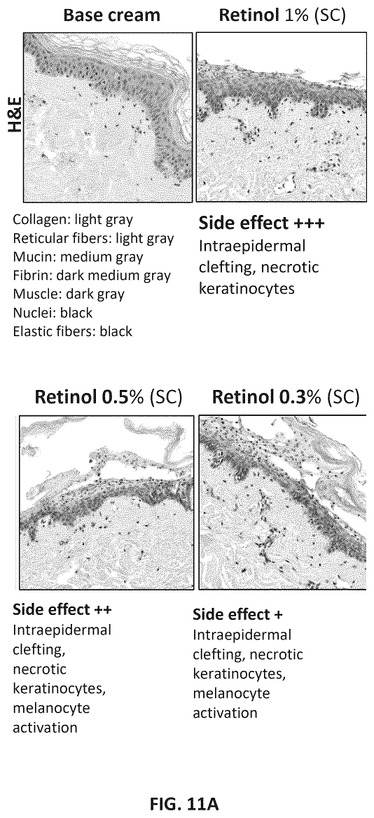

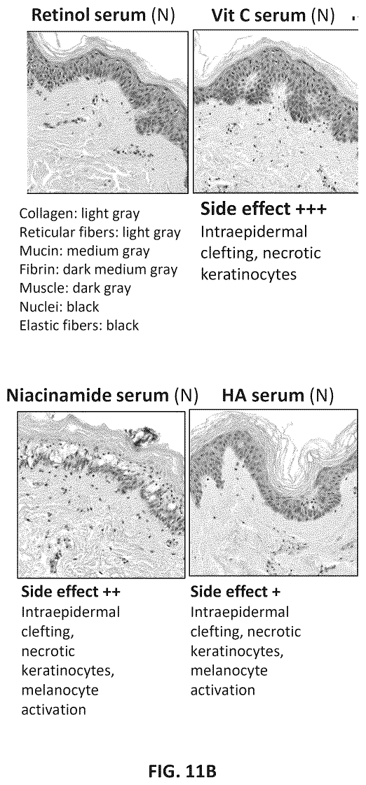

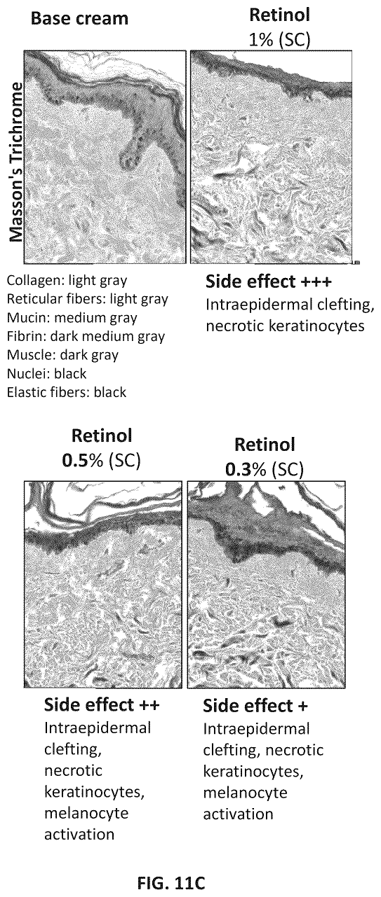

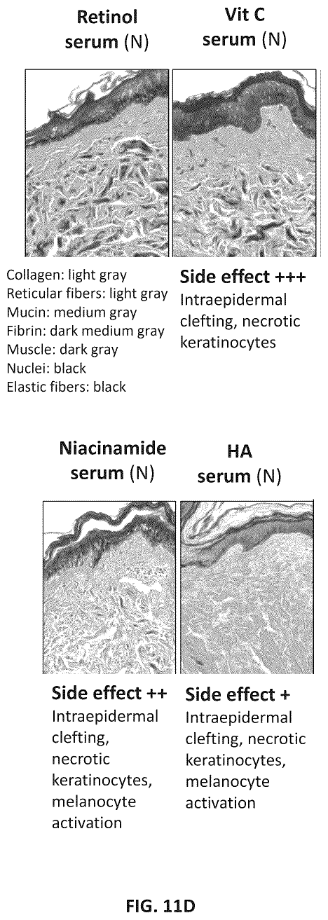

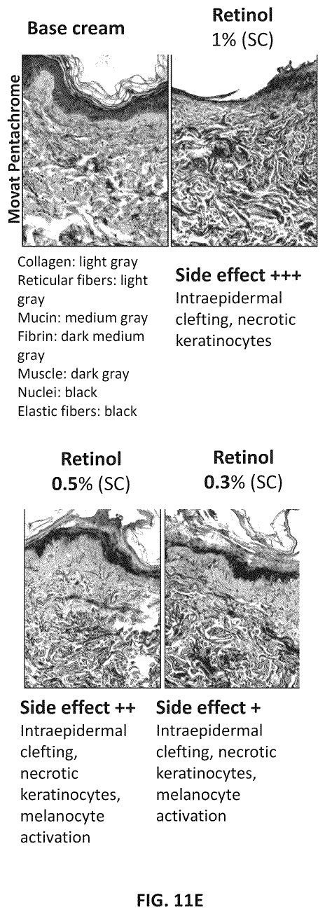

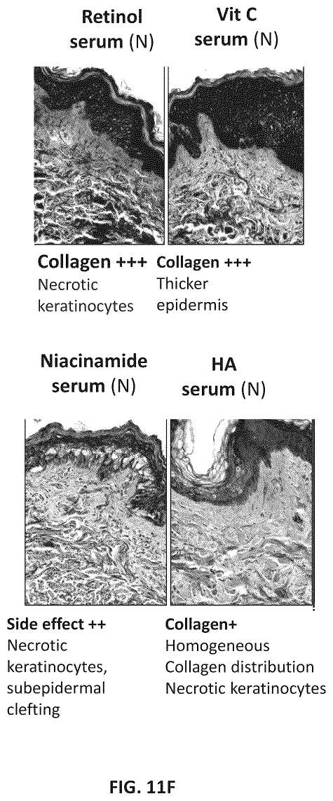

The purpose of this example is to indicate a histological analysis of tissues achieved using an HCM and HTC according to an embodiment of this disclosure. Skin tissues were grown and analyzed for various molecular markers to illustrate a resulting tissue complexity and the impact of various test agents, compounds or biologics of interest on the tissues from a histological perspective. These results provided example benchmarks for skin product testing, thereby evaluating potential efficacy or toxicology of the skin products. FIGS. 11A-11F provide a series of micrographs comprising histology samples from ex vivo tissue model samples subjected to test conditions with different stains applied (SC=skin cream: N=). FIGS. 11A-11B provide micrographs with H&E stain, where samples were treated with base cream. or retinol 1%, or retinol 0.5%, or retinol 0.3%, or retinol serum, or vitamin C serum, or niacinamide serum, or hyaluronic acid serum. In some embodiments, the retinol serum comprised bukachiol. In some embodiments, the retinol serum comprised a retinol alternative. FIGS. 11C-11D provide micrographs with Masson's Trichrome stain, where samples were treated with base cream, or retinol 1%, or retinol 0.5%, or retinol 0.3%, or retinol serum, or vitamin C serum, or niacinamide serum, or hyaluronic acid serum. FIGS. 11E-11F provide micrographs with Movat Pentachrome stain, where samples were treated with base cream, or retinol 1%, or retinol 0.5%, or retinol 0).3%, or retinol serum, or vitamin C serum, or niacinamide serum, or hyaluronic acid (HA) serum.

The results in FIGS. 11A-11F illustrate tissue complexity in both structure and viability. For example, the staining indicated features such as: elastic fibers, nuclei, collagen, reticular fibers, mucin, fibrin, muscle, cytoplasm, and keratin. For example, the retinol 1% (SC) resulted in higher magnitude side effects of intraepidermal clefting and necrotic keratinocytes; the retinol 0.5% (SC) resulted in medium magnitude side effects of intraepidermal clefting, necrotic keratinocytes, and melanocyte activation; the retinol 0.3% (SC) resulted in lower side effects of intraepidermal clefting, necrotic keratinocytes, and melanocyte activation; retinol serum (N) resulted in higher levels of collagen production and necrotic keratinocytes; the vitamin C serum (N) resulted in higher levels of collagen production and a thicker epidermis; the niacinamide serum (N) resulted in medium side effects of necrotic keratinocytes and subepidermal clefting, and the HA serum resulted in a lower level of collagen production, homogeneous collagen distribution, and necrotic keratinocytes.

### Example 3

### Gene or Protein Expression Maps

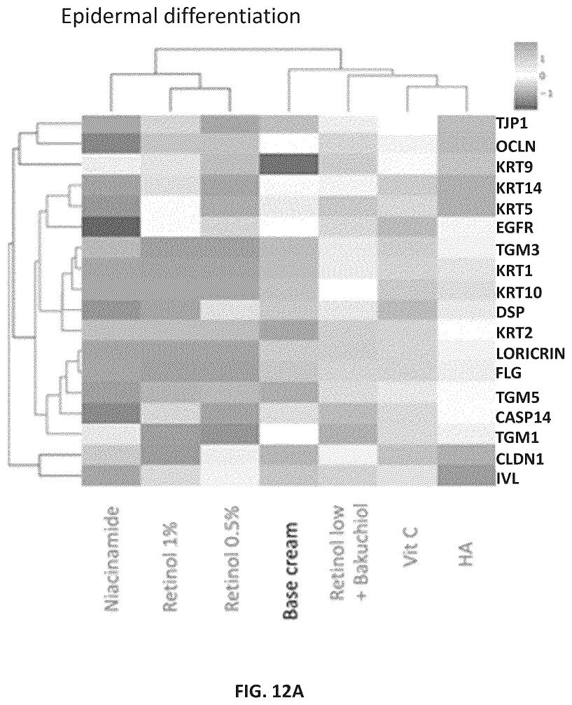

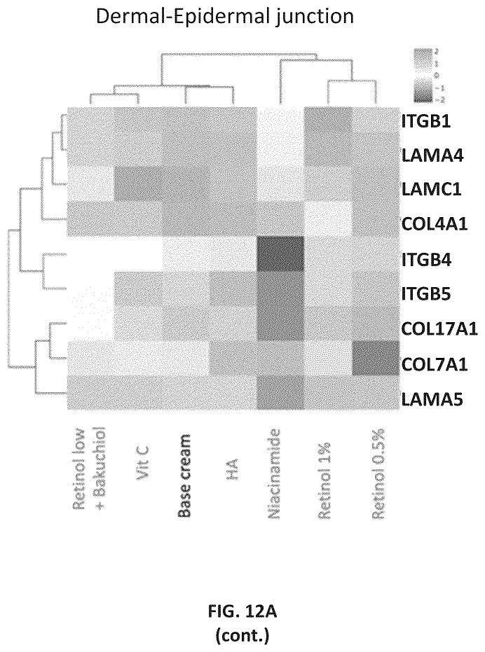

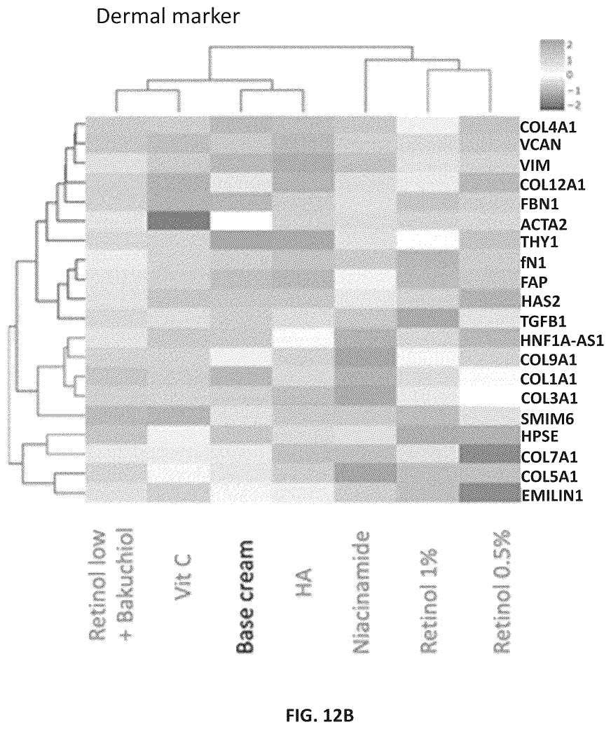

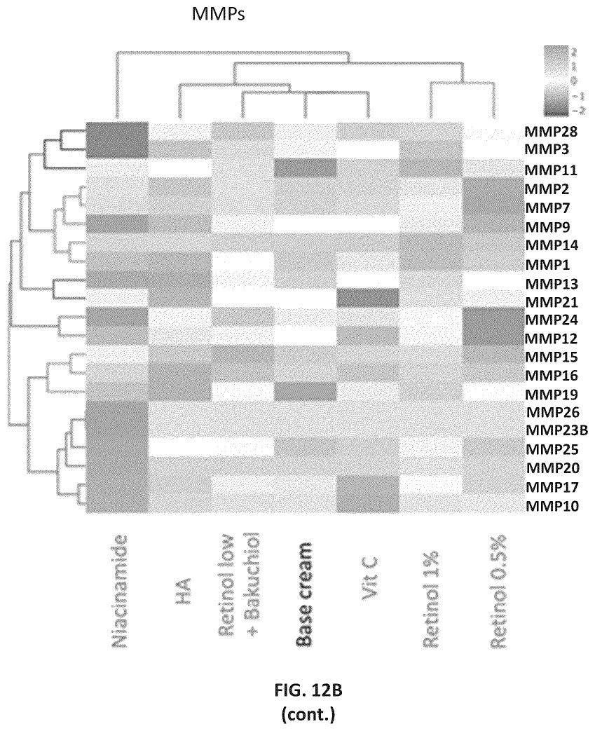

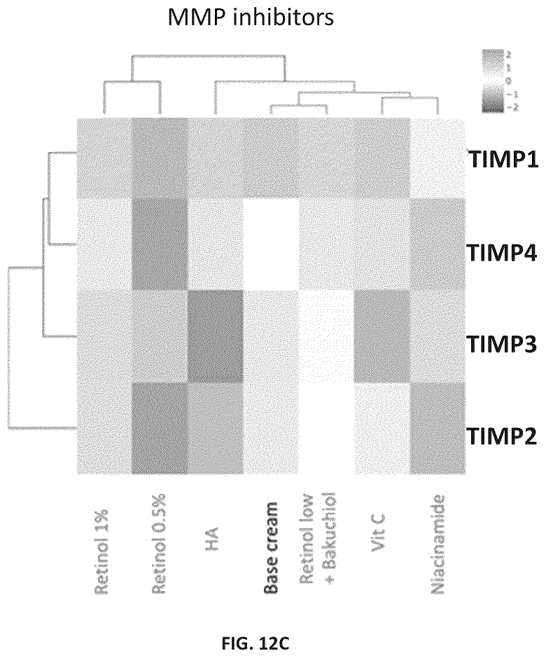

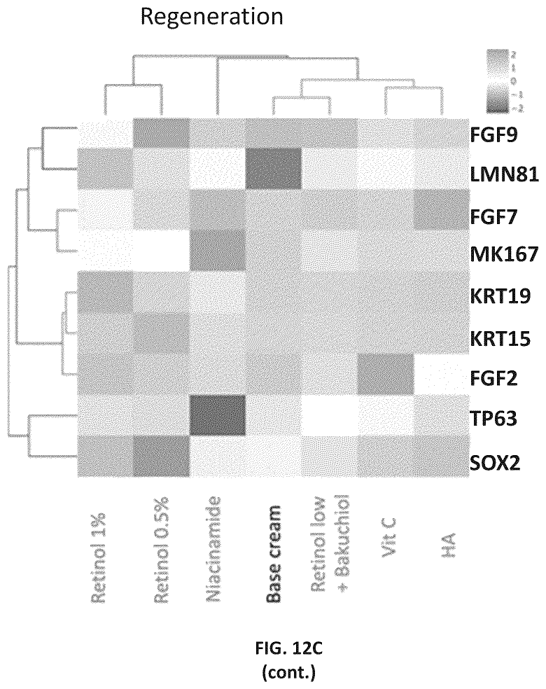

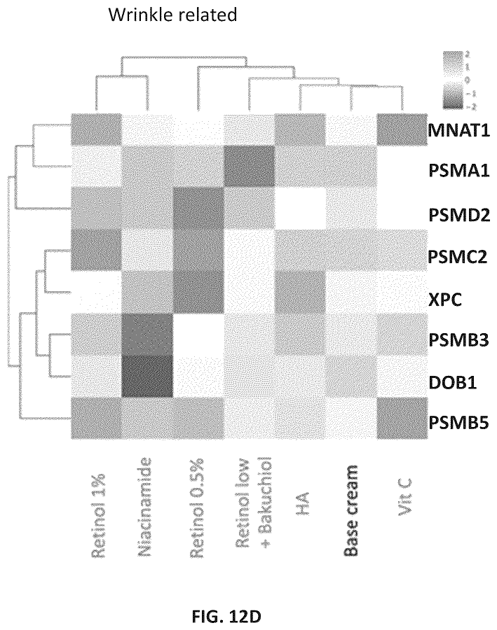

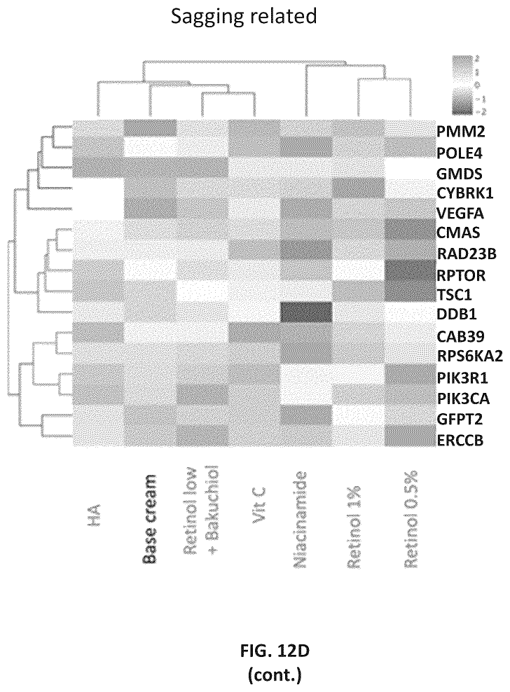

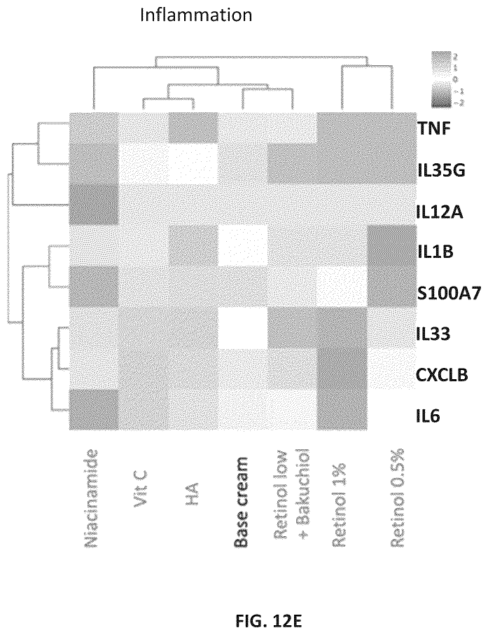

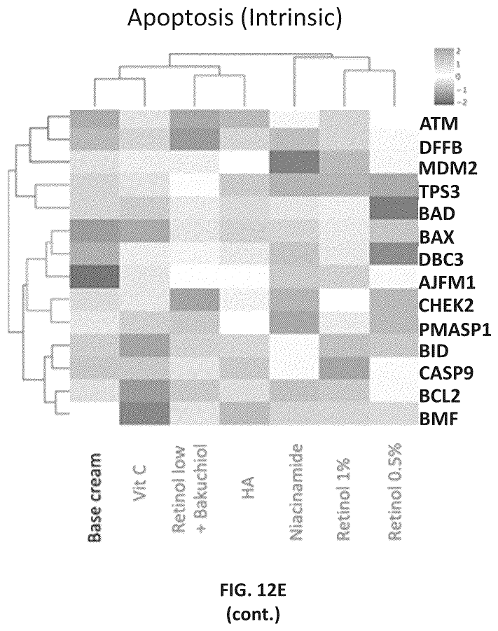

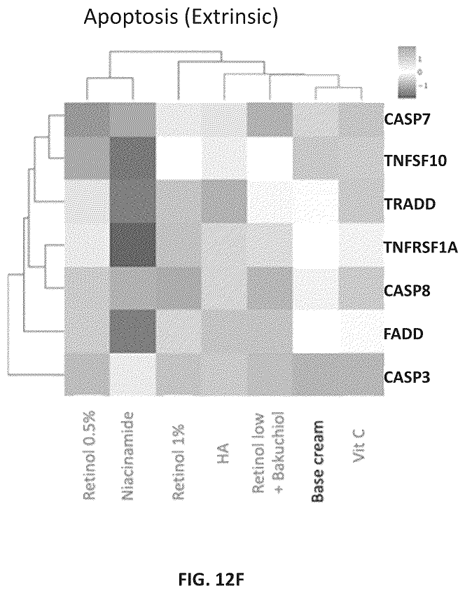

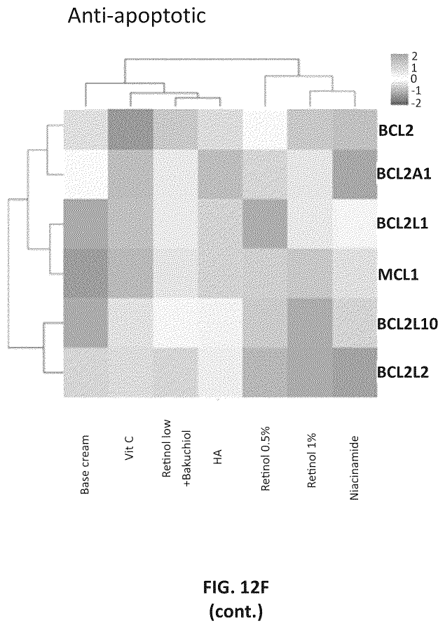

The purpose of this example is to provide a genomics analysis of tissues achieved using an HCM and HTC according to an embodiment of this disclosure. Skin tissues were grown and analyzed for various genetic markers to illustrate a resulting genetic profile of the tissue sample and the impact of various test agents, compounds or biologics of interest on the tissues based on gene mapping configuration. In particular, each set of genes expression levels indicated in the mapping illustrates resulting expression changes upon exposure to the test agents. These results provided additional example benchmarks for skin product testing, thereby evaluating potential efficacy or toxicology of the skin products. FIGS. 12A-12F are gene expression heatmaps for several different study conditions according to one or more aspects of the present disclosure. FIG. 12A provides gene expression maps for epidermal differentiation and dermal-epidermal junction, where branching on the left of each heat map indicates clustering of samples based on similarity of gene expression. FIG. 12B provides gene expression maps for dermal markers and MMPs. FIG. 12C provides gene expression maps for MMP inhibitors and regeneration. FIG. 12D provides gene expression maps for wrinkle-related genes and sagging-related genes. FIG. 12E provides gene expression maps for and inflammation and apoptosis (intrinsic). FIG. 12F provides gene expression maps for apoptosis (extrinsic) and anti-apoptotic genes. Each of the gene expression maps is quantified for each of the treatments of base cream, or retinol 1%, or retinol 0.5%, or retinol 0.3%, or retinol serum, or vitamin C serum, or niacinamide serum, or HA serum.

### Example 4

### Experimental Plan for Pathway Analysis

The purpose of this example is to provide an experimental plan for a gene pathway analysis. A cell and model source for this experimental plan is a human skin biopsy. The applications for testing are for anti-aging, vehicle testing, UV, and “improvement”.

A treatment plan for the anti-aging comprises application of retinol cream, a plurality of retinoic acid (pure) formulations (such as, for example, three retinoic acid formulations labeled RA, F1; RA, F2; and RA, F3), and a vehicle to the human skin biopsy.

A treatment plan for the UV comprises application of UV to the human skin biopsy. The ingredient conditions to be tested comprise sunscreen (prior to the treatment with UV) labeled for UV protection, a no treatment option labeled for UV damage, and a plurality of post-treatment options (such as, for example, two post-treatment options) labeled for UV damage recovery.

A treatment plan for the UV comprises no treatment, but with an application of a vehicle ingredient and a retinol ingredient.

The endpoints to be measured during each treatment plan are one or more of the following: Ultrasound, Dermoscopy, Photography Corneometer, Cutometer, TEWL, Mexameter, pH IHC staining: H&E, PAS, Trichrome, Elastin, Filaggrin, Keratin 10, Keratin 14, Ki67, Transglutaminase 1, Collagen IV, Collagen VII, Laminin 5, Fibrillin, Pro-collagen 1, HAS, Vimentin, Lipid For UV groups: Active caspase 3, TUNEL, Cyclobutanepyrimidine dimers Effluent assays: MMPs, Serine proteases, ROS, For UV groups: IL-2, 4, 6, 8, 10, 17, 18, 22, 27, TNF-α, IFN-gamma, TGH-beta, MTT Bulk RNA seq for pathway analysis, and LCMS for absorption and metabolism.

### Example 5

### Tissue Longevity Analysis

The purpose of this example is to provide an analysis of skin tissue culture longevity according to aspects of the present disclosure.

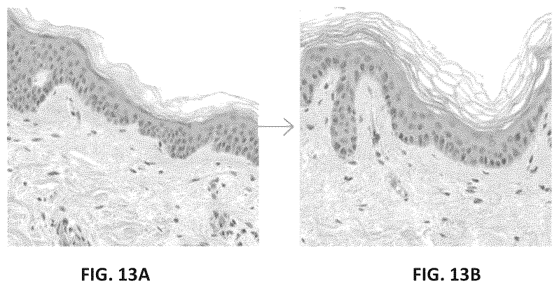

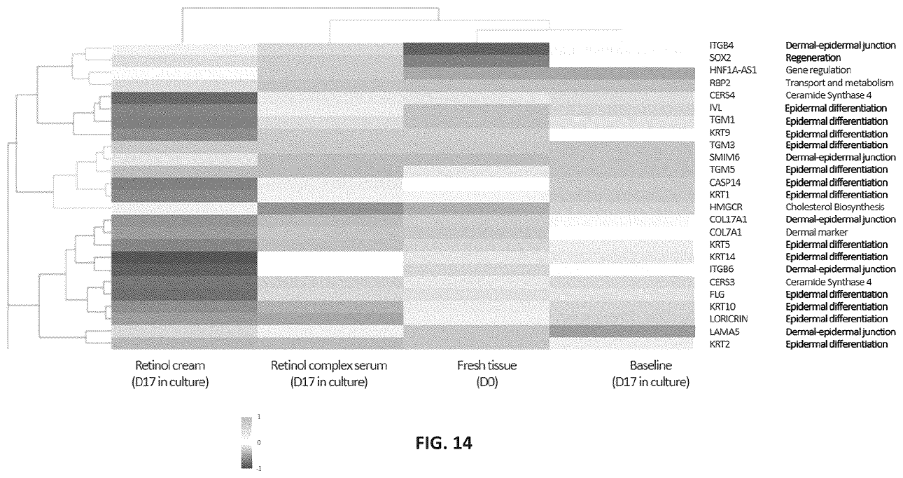

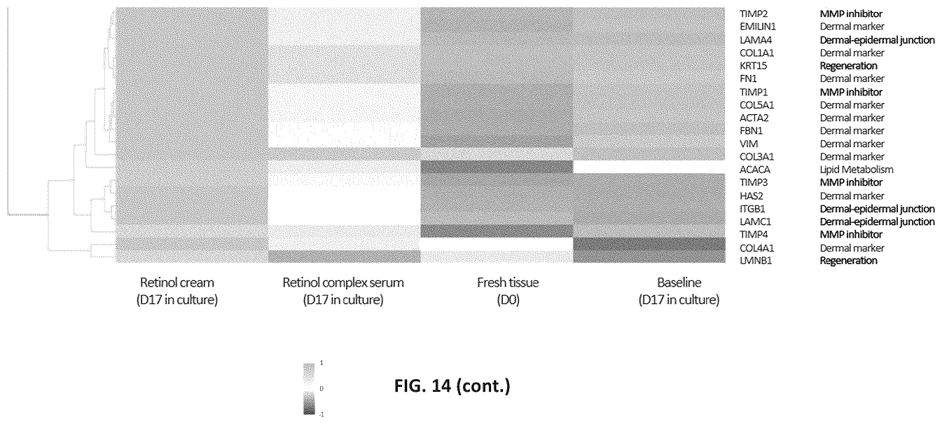

FIGS. 13A-13B provide a histology result comparison for fresh human skin tissue between day 0 and at about 3 weeks in culture, respectively, in an insert using media comprising the components in Table 4, showing proof-of-concept of tissue viability within the insert during that time interval. FIG. 14 further provides an analysis of selected genes involved in skin development and biological functions that was conducted via comparison of z-scores in an expression heatmap. As shown, skin tissue cultures of the present example had comparable gene expression levels to fresh tissue (day 0) after 17 days in culture. Retinol complex serum and retinol cream applied to the skin tissues showed distinct gene expression patterns from the baseline groups.

TABLE 4
In-house media 1 composition.
Media Component

50% CELLnTEC Airlift
Medium
16.67% PromoCell
Keratinocyte Growth Medium
2
16.67% PromoCell Fibroblast
Growth Medium 2
16.67% PromoCell Adipocyte
Nutrition Medium
50 μg/mL Ascorbic acid
1% Pen-Strep

Table 5 provides a plurality of media mixtures applied to skin samples to assess the effects of various testing conditions on the skin tissue. By analyzing histologically stained tissue sections via microscopy, the best media conditions were determined, as provided in Table 6, using the experimental criteria of epidermis and dermis structure, as well as examining the consistency between skin tissue sample groups, with two samples per group.

TABLE 5
Media mixtures

| | Break- | | | |
|---|---|---|---|---|
| Media | down | | | |
| Mix | (%) | Contents | Supplements | |
| 1 | 100 | CELLnTEC Airlift | | 1% Pen- |
| | | Media | | Strep |
| | | | | Ampho-B |
| 2 | 100 | Phenion ALI | | Ampho-B |
| 3 | 50 | Promocell Keratinocyte | | 1% Pen- |
| | | Growth Media 2 | | Strep |
| | 50 | Promocell Fibroblast | | Ampho-B |
| | | Growth Media 2 | | |
| 4 | 50 | Promocell Keratinocyte | Vit-C | 1% Pen- |
| | | Growth Media 2 | | Strep |
| | 50 | Promocell Fibroblast | | Ampho-B |
| | | Growth Media 2 | | |
| 5 | 50 | Promocell Keratinocyte | Vit-C | 1% Pen- |
| | | Growth Media 2 | Blood | Strep |
| | 50 | Promocell Fibroblast | Substitute | Ampho-B |
| | | Growth Media 2 | | |
| 6 | 100 | Williams E Culture | 1% Glutamax | 1% Pen- |
| | | Medium | 1% non- | Strep |
| | | | essential AA | Ampho-B |
| | | | solution | |
| | | | 1% ITS | |
| | | | 10 ng/mL | |
| | | | hydrocortisone | |
| 7 | 100 | Williams E Culture | 1% Glutamax | 1% Pen- |
| | | Medium | 1% non- | Strep |
| | | | essential AA | Ampho-B |
| | | | solution | |
| | | | 1% ITS | |
| | | | 10 ng/mL | |
| | | | hydrocortisone | |
| | | | Vit-C | |
| 8 | 100 | Williams E Culture | 1% Glutamax | 1% Pen- |
| | | Medium | 1% non- | Strep |
| | | | essential | Ampho-B |
| | | | AA solution | |
| | | | 1% ITS | |
| | | | 10 ng/mL | |
| | | | hydrocortisone | |
| | | | Vit-C | |
| | | | 1% Human | |
| | | | Serum | |
| 9 | 100 | Williams E Culture | 1% Glutamax | 1% Pen- |
| | | Medium | 1% non- | Strep |
| | | | essential | Ampho-B |
| | | | AA solution | |
| | | | 1% ITS+ | |
| | | | 10 ng/mL | |
| | | | hydrocortisone | |
| | | | Vit-C | |
| 10 | 100 | Williams E Culture | 1% Glutamax | 1% Pen- |
| | | Medium | 1% non- | Strep |
| | | | essential | Ampho-B |
| | | | AA solution | |
| | | | 1% ITS+ | |
| | | | 10 ng/mL | |
| | | | hydrocortisone | |
| | | | Vit-C | |
| | | | Blood | |
| | | | Substitute | |
| 11 | 100 | Williams E Culture | 1% Glutamax | 1% Pen- |
| | | Medium | 10% FBS | Strep |
| | | | | Ampho-B |
| 12 | 50 | CELLnTEC Airlift | Vit-C | 1% Pen- |
| | | Media | | Strep |
| | 16.67 | Promocell Keratinocyte | | Ampho-B |
| | | Growth Media | | |
| | 16.67 | Promocell Fibroblast | | |
| | | Growth Media | | |
| | 16.67 | Promocell Adipocyte | | |
| | | Nutrition Media | | |

TABLE 6
Media mixtures

| Media mix 1 | Media mix 3 | Media mix 6 | Media mix 8 |
|---|---|---|---|
| 100% CELLnTECH | 50% PromoCell | Base: Williams E | Base: Williams E |
| Airlift medium | Keratinocyte | Culture Medium | Culture Medium |
| | Growth Medium 2 | | |
| 1% Pen-Strep | 50% PromoCell | 1% Glutamax | 1% Glutamax |
| | Fibroblast Growth | | |
| | Medium 2 | | |
| | 1% Pen-Strep | 1% Non-essential AA | 1% Non-essential AA |
| | | solution | solution |
| | | 1% ITS+ | 1% ITS+ |
| | | 10 ng/mL | 10 ng/mL |
| | | Hydrocortisone | Hydrocortisone |
| | | 1% Pen-Strep | 50 μg/mL Ascorbic |
| | | | acid |
| | | | 1% Human Serum |
| | | | 1% Pen-Strep |

| Media mix 9 | Media mix 11 | Media mix 12 |
|---|---|---|
| Base: Williams E | Base: Williams E | 50% CELLnTEC Airlift |
| Culture Medium | Culture Medium | Medium |
| 1% Glutamax | 1% Glutamax | 16.67% PromoCell |
| | | Keratinocyte Growth |
| | | Medium 2 |
| 1% Non-essential AA solution | 10% FBS | 16.67% PromoCell |
| | | Fibroblast Growth Medium 2 |
| 1% ITS+ | 1% Pen-Strep | 16.67% PromoCell |
| | | Adipocyte Nutrition Medium |
| 10 ng/mL Hydrocortisone | | 50 μg/mL Ascorbic acid |
| 50 μg/mL Ascorbic acid | | 1% Pen-Strep |
| 1% Pen-Strep | | |

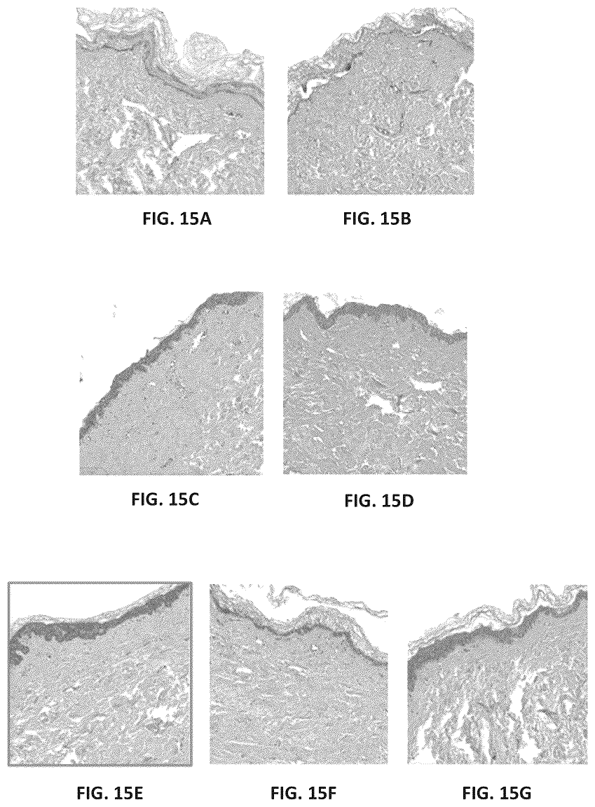

FIGS. 15A-15G illustrate histology results in using each of the media mixes of Table 6. The best media condition is shown in FIG. 15E, which was determined based on epidermis and dermis structure, and consistency between 2 replicates. Further testing conditions comprised first treating 0.1 mM of calcium chloride on the surface of skin samples, and applying higher concentrations (0.5 mM and 1 mM) of calcium chloride in media. However, neither testing condition was observed to have any beneficial effect.

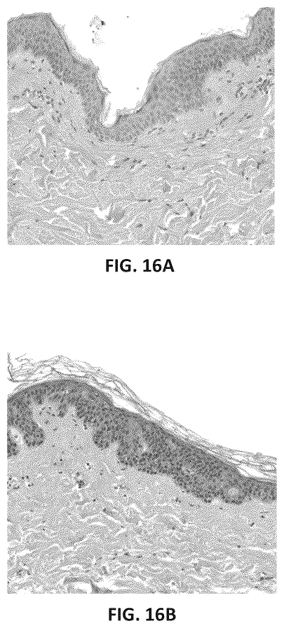

FIGS. 16A-16B illustrate histology results using media mix 12, as provided in Table 7, between day 0 and at 4 weeks in culture, respectively. Based on the epidermis and dermis structure, media mix 12 was shown to result in the longest culture duration among all of the commercial models.

The testing using the above conditions resulted in healthy, living skin morphology for up to four weeks in culture, which represents a fourfold increase over existing commercial ex vivo models. By allowing for long term treatment studies beyond conventional approaches, complex biological interactions and clinically relevant phenomena can be better understood. Studies with greater tissue culture longevity are likely to result from further experimentation.

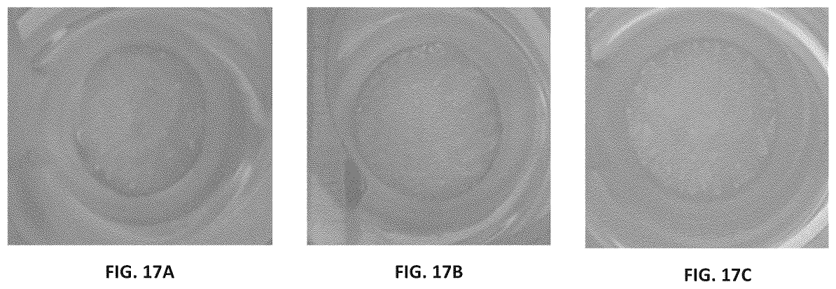

Skin tissue cultures were further observed to survive up to six weeks after seeding. FIGS. 17A-17C depict photographed images of culture samples surviving at six weeks including samples subjected to Media Mix 9, Media Mix 9 with 5% human platelet lysate, and Media Mix 9 with 5% human platelet lysate and 10ng/mL EGF and 10 ng/ml bFGF, respectively.

### Example 6

### Biopsy Sealing and Transepidermal Water Loss (TEWL) Analysis

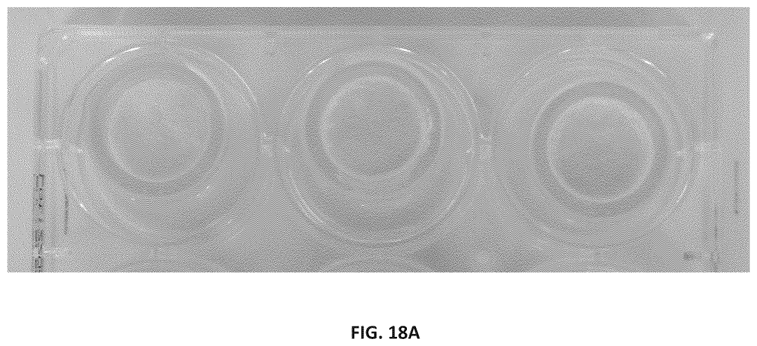

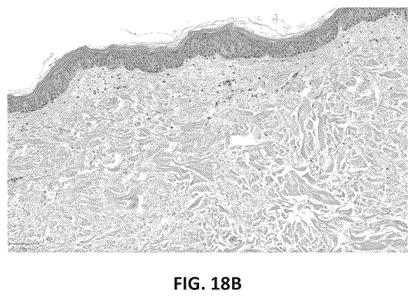

The purpose of this example is to incorporate sealed sensors into an insert. Sealing the biopsy edge using Loctite super glue, as shown in the photograph in FIG. 18A and the histology stain image in FIG. 18B, allowed the measurement of changes in barrier function with sensitivity, as well as preventing leakage of topically-applied substances into the dermis through the edge of the biopsy as well as allowing for the precise measurement of changes in the barrier function of the skin using clinical sensors. Table 7 provides adhesives tested and a corresponding result of their use.

TABLE 7
Adhesives tested for sealing tissue to insert

| Glue | Outcome |
|---|---|
| 3M Vetbond Tissue | Did not cure fast enough |
| Adhesive | |
| Eakin Cohesive | Swelled during culture |
| Loctite Superglue | Adequate curing observed |
| Pros-Aide Glue | Did not cure fast enough |
| Gorilla Glue | Did not cure sufficiently |

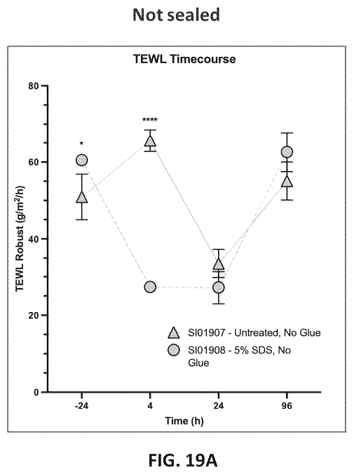

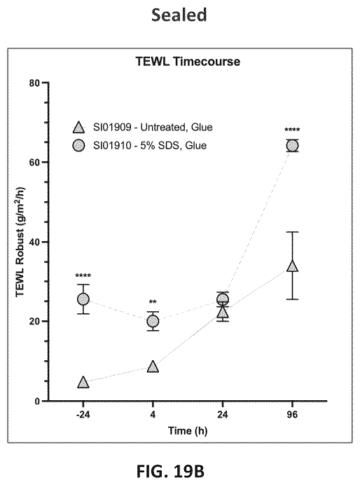

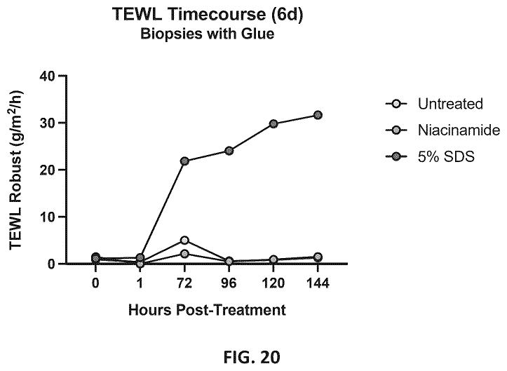

FIGS. 19A-19B illustrate measurements of transepidermal water loss (TEWL) in model skin that were either not sealed or sealed using Loctite super glue, respectively. TEWL measurements were taken using a Courage+Khazaka Multi Probe Adapter MPA 10. Accurate measurement of TEWL is facilitated by sealing the edge of the biopsy to the insert; this sealing was observed to enhance the sensitivity of detecting changes in the barrier function of the skin. For example, FIGS. 19A-19B show that the expected reduction in the barrier function caused by SDS treatment was only observable via TEWL sensing after sealing of biopsy edges. FIG. 20 show a comparison in TEWL measurements of the effects of a barrier enhancer (Niacinamide) and a barrier disrupter (SDS) on skin sealed with Loctite super glue over a six-day period.

### Example 7

### Exemplary Data Mapping Analysis

The purpose of this example is to provide a visualization of data maps using data from exemplary test agents. FIG. 21A illustrates exemplary data types that contribute to data maps, while FIGS. 21B-21C illustrate a comparison between data maps resulting retinol and a retinol alternative, respectively. The data maps of FIG. 21A resulted from multi-modal data using particular thresholds for each data type. The data comprised, for example, gene set enrichment analysis (GSEA) based on a Net Enrichment Score (NES) threshold of 1.2 or more, differential gene expression data (DGE) based on a Log Fold Change (logFC) threshold of 5 or above, a pathology score based on an improvement threshold from a vehicle of 20% or more, and lactate dehydrogenase (LDH) release data based on a percent release threshold relative to a lysed control. Table 9 provides a list of exemplary histopathological parameters used for scoring the compiled experimental data from the test agents for their respective evaluation, where the scores are used as pathology score inputs into the data maps. Table 9 may also be used to assess viability of the tissues and/or cells. In some embodiments, skin tissue samples may be considered viable when each of epidermal damage, eczematous dermatitis, hyperpigmentation, thickness of epidermis, solar elastosis, and collagen deposition each score 1 when compared to a day 0 control skin tissue sample. In some embodiments, skin tissue samples may be considered viable when, in comparison to the day 0 control skin tissue sample, each of an epidermal damage score does not exceed 2, an eczematous dermatitis score does not exceed 1, and scores for hyperpigmentation, solar elastosis, and collagen deposition do not exceed 1.

The resulting data maps illustrate a 4-fold increase in skin barrier function and a two-fold decrease in irritation when using the retinol alternative as compared to the retinol.

TABLE 9
Histopathological parameters for data mapping

| | Epidermal damage | Eczematous | |
|---|---|---|---|
| Score | (chemically irritated) | dermatitis (atopic) | Hyperpigmentation |
| 1 | Normal | Absence | Few melanin pigments |
| 2 | Focal dyskeratotic | Minimal | Focal melanin pigments identified |
| | cells | | in high power view |
| 3 | Multifocal | Mild | Focal melanin pigments identified |
| | dyskeratotic cells | | in low power view |
| 4 | Diffuse dyskeratotic | Moderate | Continuous melanin pigments |
| | cells | | identified in high power view |
| 5 | Multifocal | Severe | Continuous melanin pigments |
| | dyskeratotic cells | | identified in low power view or |
| | and/or epidermal | | dermal melanophges |
| | blister | | |
| 6 | Diffuse dyskeratotic | | Continuous melanin pigments |
| | cells and epidermal | | identified in low power view and |
| | blister | | dermal melanophges |
| 7 | | | |
| | Thickness of epidermis | | |
| | (atrophy, acanthosis, | | |
| Score | physically irritated) | Solar elastosis | Collagen deposition |
| 1 | <25 um and/or loss of rete | Absence | Diffuse sparse |
| | ridge (severe) | | |
| 2 | 25-50 um and shortened rete | Minimal degenerated | Multifocal sparse |
| | ridge (<epidermis) (mild) | collagen: <10% | |
| 3 | 25-50 um or shortened rete | Mild degenerated | Focal sparse |
| | ridge (<epidermis) (mild) | collagen: 10-30% | |
| 4 | 50-99 um and/or shortened | Moderate degenerated | Minimal sparse |
| | rete ridge (<epidermis) | collagen: 30-60% | |
| | (minimal) | | |
| 5 | 100-150 um (normal) | Severe degenerated | Focal deposition |
| | | collagen: 60-90% | |
| 6 | 150-200 um | Nearly total degenerated | Multifocal deposition |
| | | collagen: >90% | |
| 7 | >200 um | | Diffuse deposition |

### Example 8

### Exemplary Endothelial Cell Growth Analysis in Porous Layer

The purpose of this example is to evaluate the use of a 3D-printed gyroid support structure in an experimental plan for the incorporation of endothelial cells. HDMECs were obtained from PromoCell and seeded at a density of 5.383e+4 cells/insert or 7.69e+5 cells/mL on Transwell inserts with 0.4 um pore size. The inserts were coated with different ECM compositions as indicated in Table 10, then imaged with a CD31 stain after a 14-day culture.

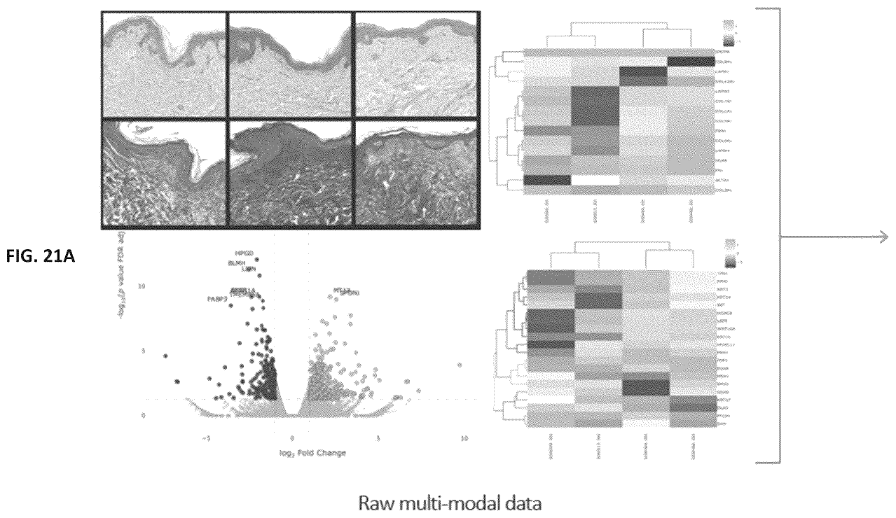

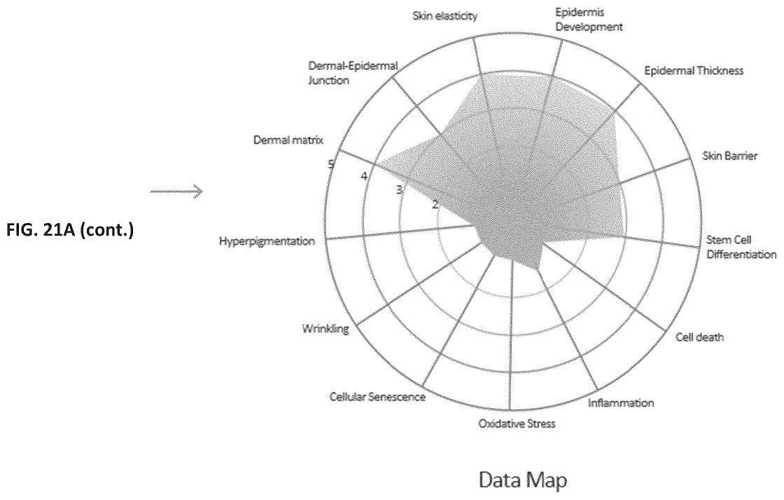

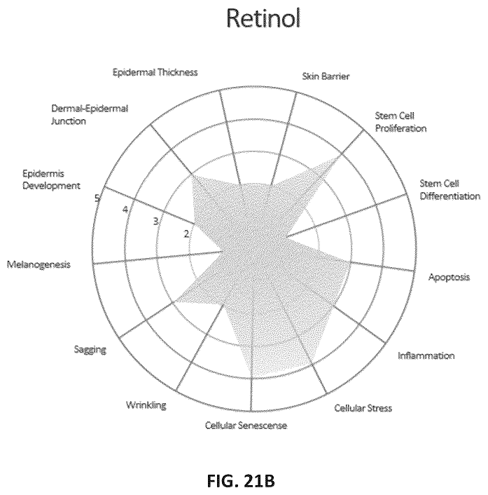

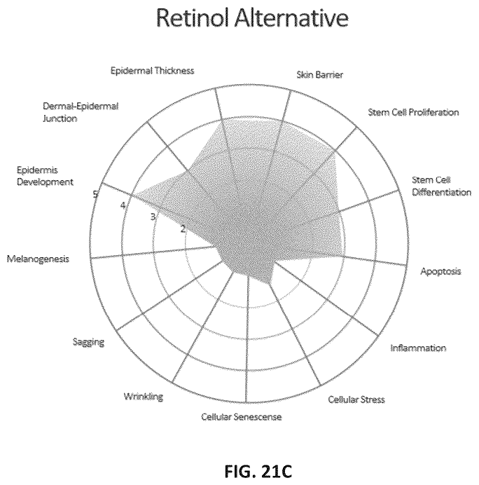

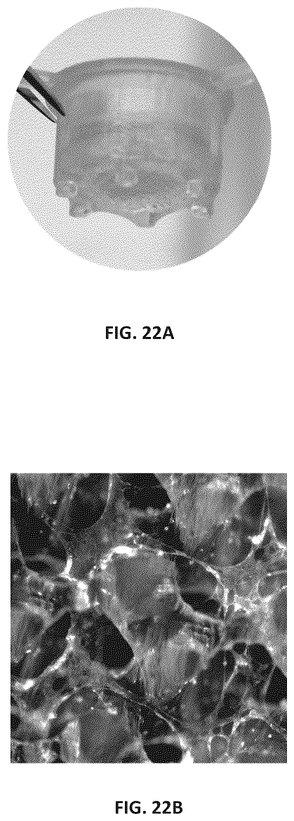

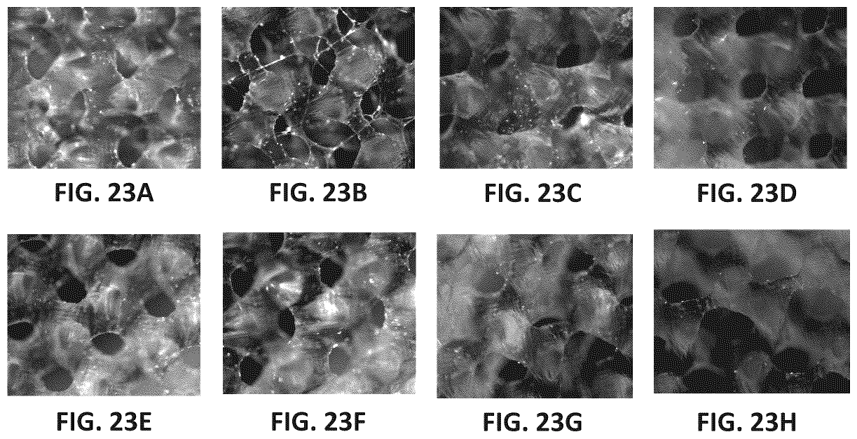

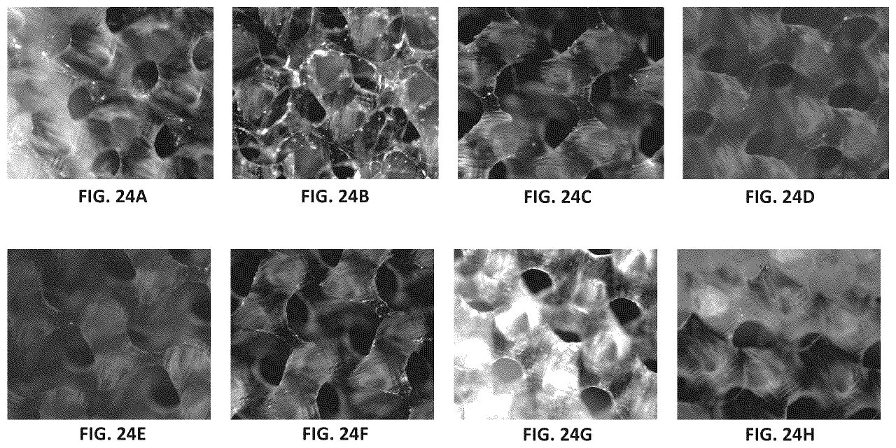

FIGS. 22A-22B provide images of cells coated on a 3D-printed support structure. FIGS. 23A-23H provide images of inserts coated with conditions 1-8 of Table 10, respectively, after a 14-day culture in CELLnTEC-Promocell media mix (Media mix 12 as described in Table 7) according to the manufacturer's instructions. FIGS. 24A-24H provide images of inserts coated with conditions 1-8 of Table 10, respectively, after a 14-day total culture, where cells were pre-treated with Endothelial Growth Media for 8 days and then switched to CELLnTEC-Promocell Media Mix for 6 days. No significant changes were observed between the two media groups.

TABLE 10
Conditions for endothelial cell network on support structure

| # | Endothelial Growth Media |
|---|---|
| 1 | 3D: ObaGel ECM from Obatala |
| 2 | 3D: ObaGel/ObaVate configuration (24 hours for gelation) from |
| | Obatala |
| 3 | 3D: Fibrinogen (2 mg/mL) + Aprotinin (1 ug/mL) + Geltrex (40 |
| | ug/ml) + Thrombin* (0.5 U/mL) |
| 4 | 3D: Fibrinogen (2 mg/mL) + Aprotinin (5 ug/mL) + Geltrex (40 |
| | ug/ml) + Thrombin* (0.5 U/mL) |
| 5 | 2D coating: Collagen IV 400 ug/mL, FN 100 ug/mL, Geltrex 50 |
| | ug/mL |
| 6 | 2D coating: Collagen IV 200 ug/mL, FN 50 ug/mL, Geltrex 50 |
| | ug/mL |
| 7 | 2D coating: Collagen I 100 ug/mL, Collagen IV 200 ug/mL, FN |
| | 50 ug/mL, Geltrex 50 ug/mL |
| 8 | Control (Uncoated) |

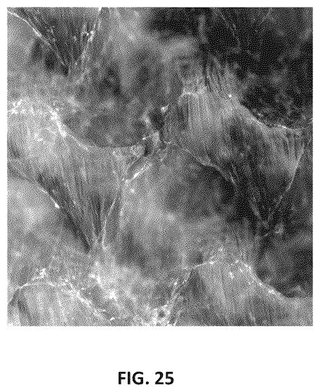

FIG. 25 provides an image of an F-actin stained insert comprising the incorporation of adipose-derived stem cells in a 3D-printed gyroid support structure, wherein human adipose-derived stem cells (HASCs) from Obatala were seeded at a density of 10⁴ cells per 100 μL and cultured for 18 days. Cells were kept in CELLnTEC and Promocell media mix (Media mix 12), and ObaGel was used as a culturing matrix. ObaGel was found to increase endothelial vascular network formation compared to other tested ECM conditions.

### Example 11

### Exemplary Skin Inflammation Analysis

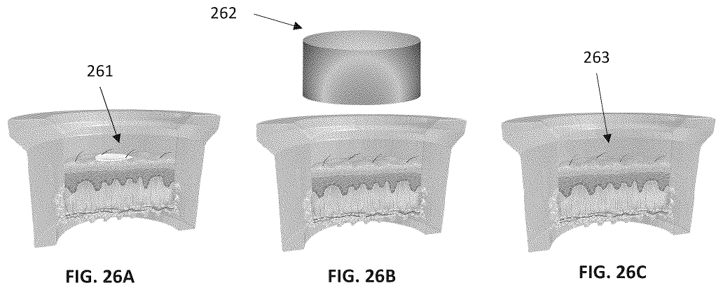

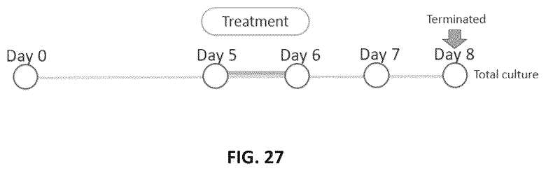

The purpose of this example is to test and replicate dynamic changes in skin across various types of clinically relevant stimuli, including skin irritations, intradermal injection. cosmetic product treatment, and antioxidant defense. Approximations of skin irritation evaluated in this manner include topical chemical treatment 261, burn treatment 262, and UV treatment 263, as illustrated in FIGS. 26A-26C, respectively. Chemical irritation was approximated through application of 2% SDS for 24 hours, burns were approximated through 100° C, contact with a heated metal rod for 45 seconds, and UV irritation was approximated via UVA and UV application for 30 minutes. FIG. 27 provides an exemplary protocol indicating that each stimulus was applied between days 5 and 6 of an 8-day culture using normal skin tissue in an insert. Skin tissues were evaluated and scored using the parameters in Table 9 as provided above. Include protocol, histological parameters, histology staining and scoring results, immune and inflammatory responses, antioxidant defense assessment, etc.

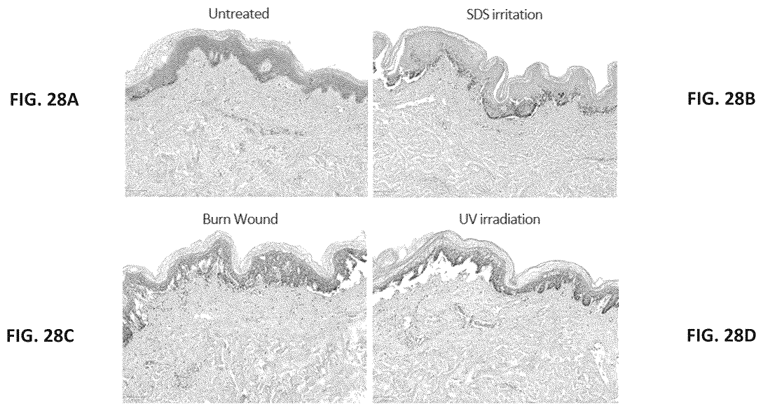

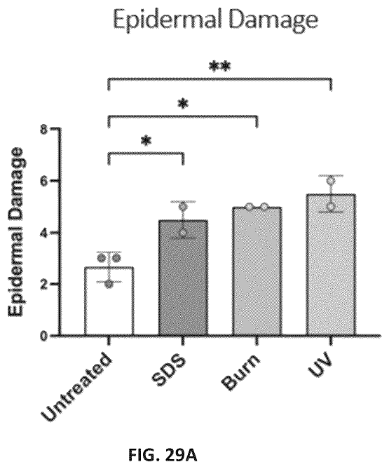

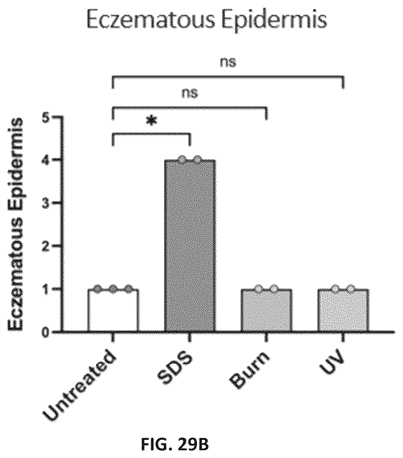

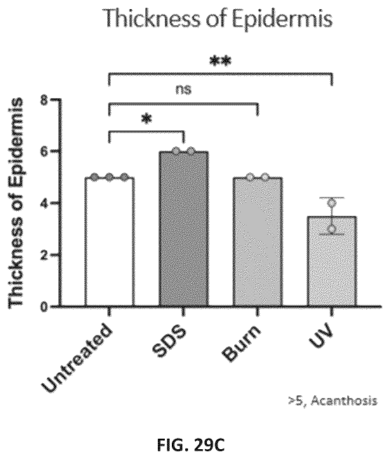

FIGS. 28A-28D provide H&E stained tissues for a first selection of untreated. chemical-treated, burn-treated, and UV-treated skin tissue samples, respectively. A pathological analysis of the extent of damage was undertaken and is shown in FIGS. 29A-29C to evaluate epidermal damage, eczematous epidermis, and epidermis thickness, respectively, after application of damage stimuli. The application of 2% SDS resulted in significant epidermal as evidenced by widespread dyskeratotic cells, moderate eczematous (atopic) epidermis, and pathological epidermal thickening (acanthosis) compared to the control group. Both the burn and UV irradiation groups exhibited significant epidermal damage. UV irradiation caused epidermal thinning compared to the control group.

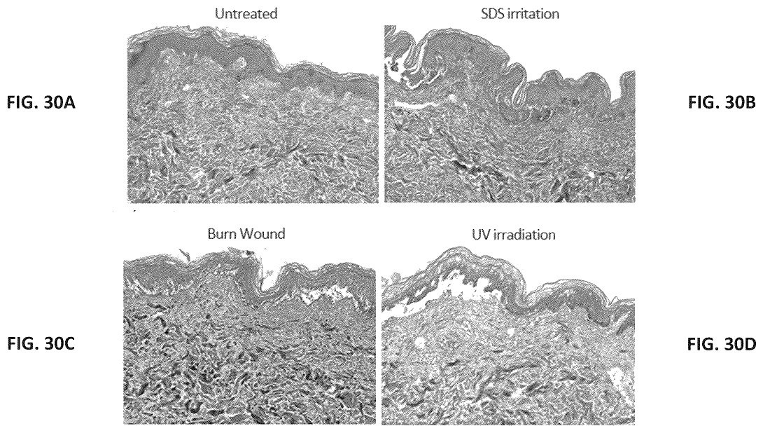

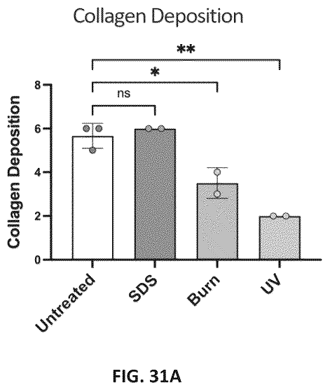

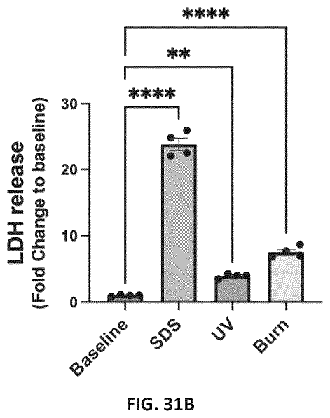

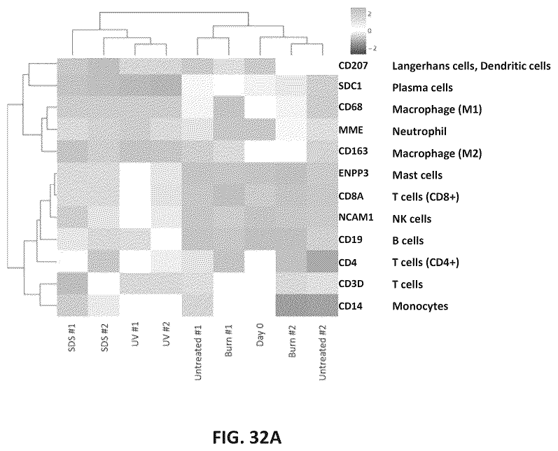

A second selection of samples were cultured and subjected to irritating stimuli, whereby a chemical stimulus group was subjected to 2% SDS for 24 hours, a burn group was subjected to 100° C, contact for 90 seconds, and a UV irritation group was subjected to UV-B for 30 minutes. FIGS. 30A-30D illustrate insert images after staining with Masson's Trichrome for untreated, chemical-treated, burn-treated, and UV-treated skin tissues, respectively. FIG. 31A provides an analysis of collagen deposition using histopathological scoring to determine the effects of damage stimuli on each group. As a result of this analysis, both burn and UV irradiation groups were discovered to have reduced collagen deposition compared to the untreated group. No significant change in collagen deposition was observed in the SDS-treated group. FIG. 31B provides an analysis of lactate dehydrogenase (LDH) release, which is widely used as a marker for assessing cell death and toxicity. The analysis indicated a significant increase of LDH release from all treatment conditions compared to untreated samples. The highest LDH release resulted from the SDS-treated group.

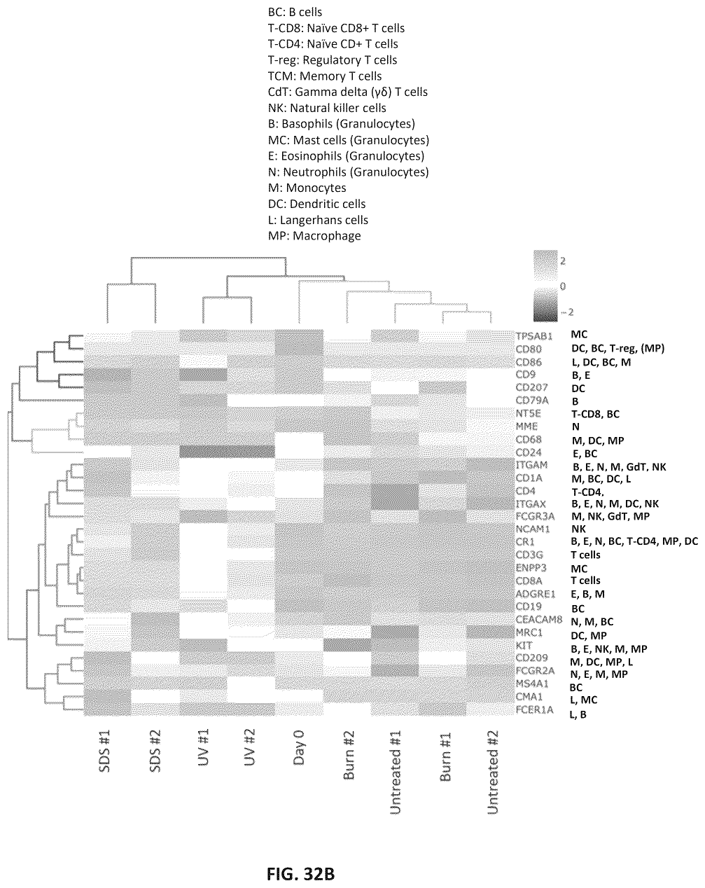

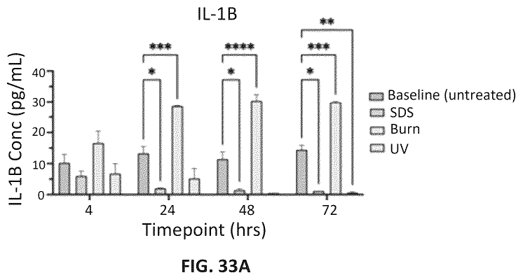

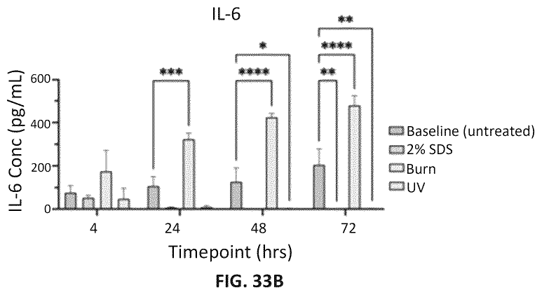

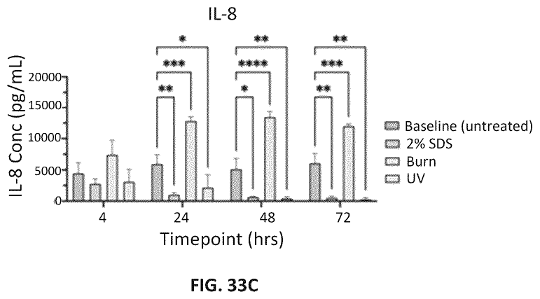

Inflammatory responses were performed and recorded for each of the first and second selection of samples for the different treatment groups. FIGS. 32A-32B provides gene expression heat maps for each of the first and second selection of samples for the different treatment groups. The heat map of FIG. 32A includes genes indicative of Langerhans cells, dendritic cells, plasma cells, MI and M2 macrophages, neutrophils, Mast cells, T cells (CD8+, CD4+, regulatory), natural killer (NK) cells, B cells, and monocytes. The heat map of FIG. 32B includes further genes indicative of memory T cells, Gamma delta T cells (GdT), and eosinophils. FIGS. 33A-33C further provide an immune cell profile of IL-1β, IL-6, and IL-8, respectively, at different time points for each of the treatment groups. From this study. increased pro-inflammatory cytokines were found to release after samples were subjected to burns relative to other treatment groups.

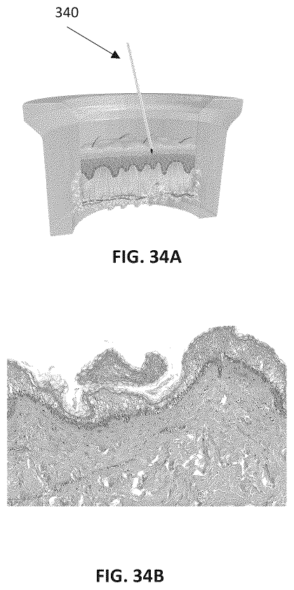

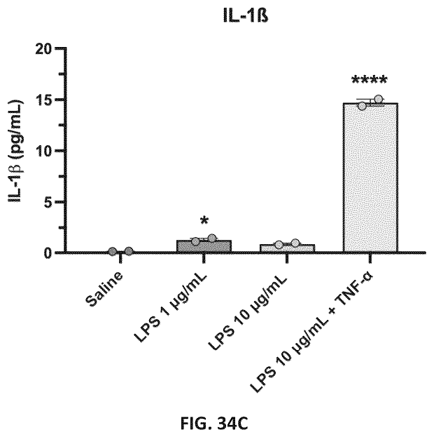

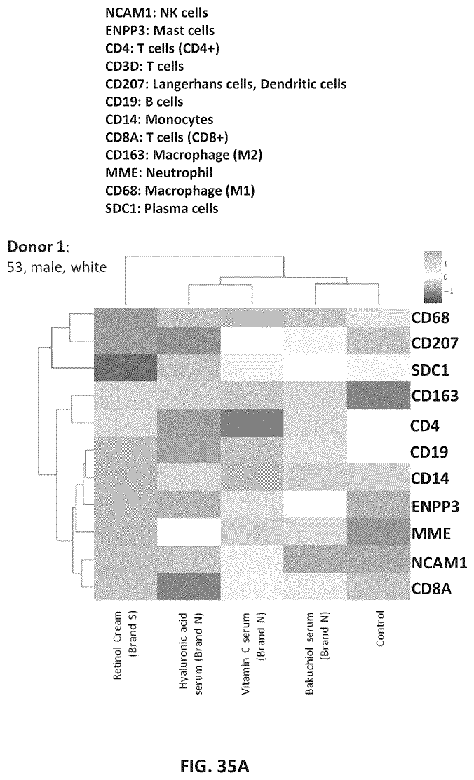

Immune cell profiling was further performed on skin tissue samples treated, using the protocol of FIG. 27, with intradermal injection 340 of lipopolysaccharide (LPS) treatments as illustrated in FIG. 34A. FIG. 34B provides an exemplary H&E stain of intradermal injection of 1 μg/mL LPS and 100 ng/ml of TNF-α. FIG. 34C provides an immune cell profile of IL-1β resulting from 1 μg/mL LPS, 10 μg/mL LPS, and 1 μg/mL LPS+TNF-α. IL-1β is a proinflammatory cytokine that is central for host responses to infection. To optimize acute and localized inflammatory conditions, various concentrations of LPS and TNF-alpha were tested. The observed 14.7 fold increase in IL-1β level in the LPS+TNF-α treated group compared to saline control treatment group indicated the onset of inflammatory response.

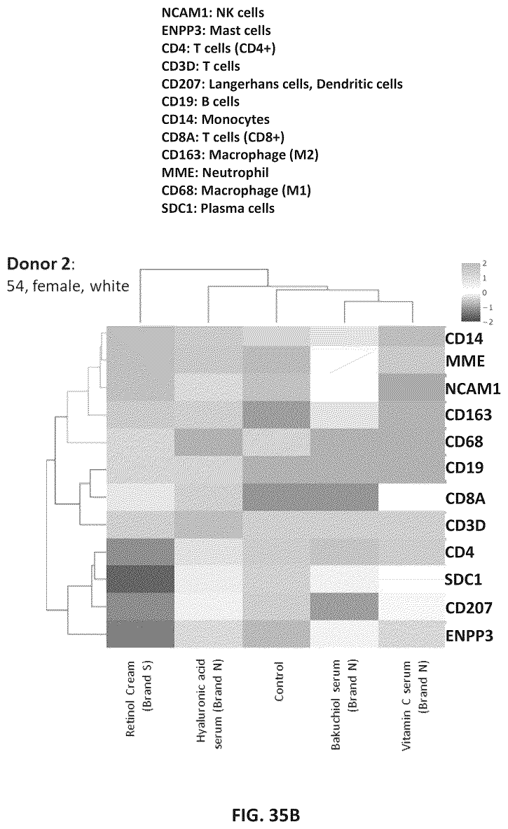

Immune cell profiling was further performed on skin tissue samples treated, using the protocol of FIG. 27, with various cosmetic products. Exemplary heat maps of the profiling are shown in FIGS. 35A-35B for two different donors, i.e., for a 53 year-old white male and a 54 year-old white female, respectively. Examples of such cosmetic products include retinol cream (0.3% Skinceutical), Hyaluronic acid serum (Natrium), and Vitamin C serum (Natrium) as compared to untreated and day 0 controls. The expression patterns of various immune cell markers were evaluated following the treatments of 4 different cosmetic products on skin samples from the donors. Both donors showed a pronounced increase in markers of T cells, monocytes, M2 macrophage, B cells, NK cells, and neutrophils compared to other treatments and control. This is an example of expression pattern analysis based on the various treatments and specific donors.

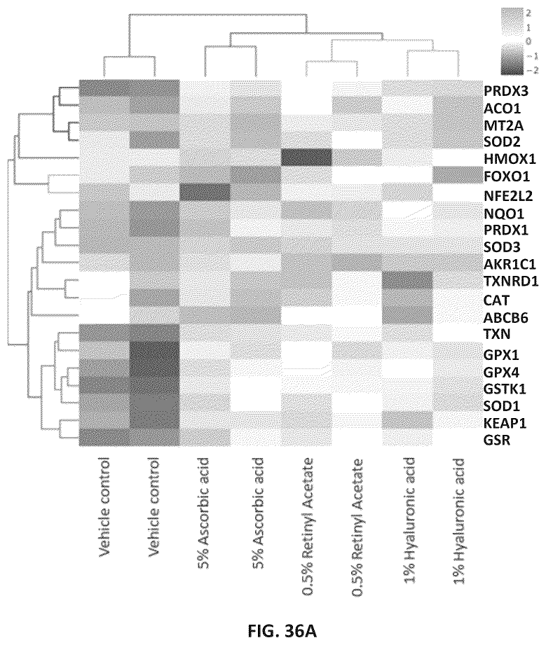

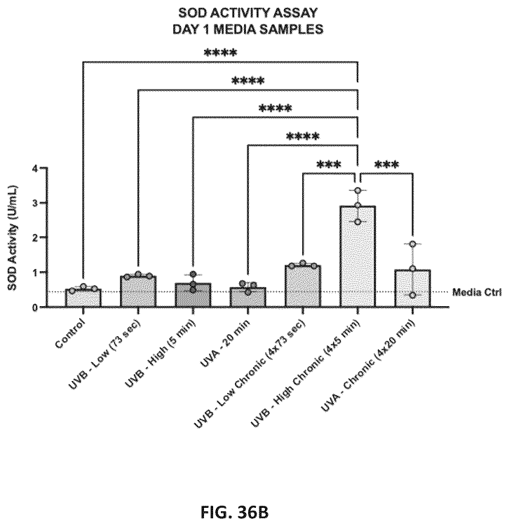

Immune cell profiling was further performed on skin tissue samples treated, using the protocol of FIG. 27, with various skincare ingredients or UV-treatments for assessing antioxidant defense in the skin. FIG. 36A provides a heat map of the assessed skincare ingredients, which include 5% Ascorbic Acid, 0.5% Retinyl Acetate, and 1% Hyaluronic Acid. FIG. 36B provides a profile of superoxide dismutase (SOD) activity, from each of various UV treatments, including UVB for 73 seconds, UVB for 5 minutes, and UVA for 20 minutes, as well as chronic treatment conditions such as UVB for 4× 73 second intervals, UVB for 4× 5 minute intervals, and UVA for 4× 20 minute intervals. The profiling in FIG. 36B illustrates a significant effect of chronic UVB exposure for longer periods of time on SOD activity, which implies a greater need for catalyzing the dismutation of superoxide radicals into molecular oxygen and hydrogen peroxide.

### Example 12

### Exemplary Protocol for Perfusion Controller

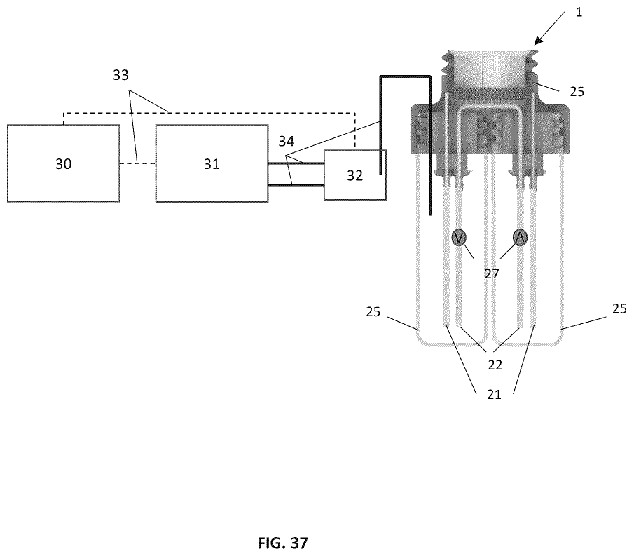

The purpose of this example is to provide an exemplary configuration and protocol for operating the perfusion controller in FIG. 1G in connection with the ex vivo skin tissue system in FIGS. 1C-1D. FIG. 37 illustrates a diagram of a configuration of the perfusion controller 30 relative to the ex vivo skin tissue system 1, the configuration further comprising an air pump 31 with a closed-loop pressure feedback system, a 3-way valve 32, and connections (e.g., electrical wire 33, pneumatic tube 34) therebetween. The pneumatic tube 34 may either be for applying positive pressure or vacuum.

Based on UI inputs, the perfusion controller 30 sends pressure setpoint and time duration to air pump 31. The air pump 31 supplies vacuum or positive pressure to the conical tube and pulls fluid through insert 10. Exemplary protocol for operation: vacuum pressure 30 seconds on, 30 seconds off, repeat for 6 hours, positive pressure for 2 minutes (to send collected effluent back to input tube), repeat entire sequence.

### DEFINITIONS

As used herein, “about” and its grammatical equivalents in relation to a reference numerical value and its grammatical equivalents as used herein can include a range of values plus or minus 10% from that value. For example, the amount “about 10” encompasses amounts from 9 to 11. The term “about” in relation to a reference numerical value can also include a range of values plus or minus 10%, 9%, 8%, 7%, 6%, 5%, 4%, 3%, 2%, or 1% from that value.

As used herein, a “cell” refers to a biological cell. Some non-limiting examples include: a prokaryotic cell, eukaryotic cell, a bacterial cell, an archaea cell, a cell of a single-cell eukaryotic organism, a protozoa cell, a cell from a plant, an algal cell, a fungal cell, a fungal protoplast cell, an animal cell, and the like. Sometimes a cell is not originating from a natural organism, e.g., a cell can be a synthetically made, sometimes termed an artificial cell.

Although various features of the disclosure may be described in the context of a single embodiment, the features can also be provided separately or in any suitable combination. Conversely, although the disclosure may be described herein in the context of separate embodiments for clarity, various aspects and embodiments can be implemented in a single embodiment.

While exemplary embodiments of the present disclosure have been shown and described herein, it will be obvious to those skilled in the art that such embodiments are provided by way of example only. Numerous variations, changes, and substitutions will now occur to those skilled in the art without departing from the present disclosure. It should be understood that various alternatives to the embodiments described herein, or combinations of one or more of these embodiments or aspects described therein may be employed in practicing the present disclosure. It is intended that the following claims define the scope of the present disclosure and that methods and structures within the scope of these claims and their equivalents be covered thereby.

1. An ex vivo skin tissue system, comprising:
an insert comprising at least one opening and a wall:
a skin tissue sample sealed inside the insert; and
a porous layer positioned inside the insert, wherein the porous layer has a first side facing the at least one opening and a second side facing opposite the at least one opening of the insert, wherein at least one pore of the porous layer comprises a gyroid, and wherein the porous layer comprises an arrangement relative to the skin tissue sample so as to promote mass transport of reagents and/or fluid into the skin tissue sample for supporting viability of the skin tissue sample for at least one week.

2. The ex vivo skin tissue system of claim 1, further comprising an adipose layer positioned inside the insert, wherein the porous layer is in contact with at least one of the adipose layer and the skin tissue sample.

3. The ex vivo skin tissue system of claim 2, wherein the adipose layer comprises human tissue from a same source as the skin tissue sample.

4. The ex vivo skin tissue system of claim 2, wherein the adipose layer comprises human adipose-derived stem cells, adipocytes, or pre-adipocytes.

5. The ex vivo skin tissue system of claim 1, wherein the gyroid comprises a gyroid infill.

6. The ex vivo skin tissue system of claim 1, further comprising at least one of an inlet channel and outlet channel positioned lateral to the porous layer.

7. The ex vivo skin tissue system of claim 1, further comprising a gel layer in contact with the second side, wherein the gel layer is configured to receive one or more of adipose-derived cells, dermal cells, and endothelial cells.

8. The ex vivo skin tissue system of claim 1, further comprising a cell composition seeded within the porous layer between the first side and the second side.

9. The ex vivo skin tissue system of claim 8, wherein the seeded cell composition comprises one or more of endothelial cells, primary cells, iPSCs, keratinocytes, fibroblasts, melanocytes, resident immune cells, circulating immune cells, stem cells, adipocytes, microbes, or a combination thereof.

10. The ex vivo skin tissue system of claim 1, wherein the skin tissue sample comprises at least one of an epidermis layer, a dermis layer, and a hypodermis layer.

11. The ex vivo skin tissue system of claim 1, wherein the porous layer comprises a porosity gradient of at least one gyroid, and wherein the porosity gradient comprises a lower density of pores in a center of the porous layer relative to an outer boundary of the porous layer.

12. The ex vivo skin tissue system of claim 1, wherein an interior surface of the wall perpendicular to the first side of the porous layer comprises a plurality of pores.

13. The ex vivo skin tissue system of claim 12, wherein the plurality of pores penetrate a desired thickness of the wall.

14. The ex vivo skin tissue system of claim 1, wherein the skin tissue sample is cultured, thereby forming an ex vivo skin model.

15. An ex vivo skin tissue system, comprising:
an insert comprising at least one opening and a wall;
a skin tissue sample and an adipose layer positioned inside the insert;
an adhesive film configured to at least contact or seal the adipose layer within the insert; and
a porous layer positioned inside the insert, wherein the porous layer has a first side facing the at least one opening and a second side facing opposite the at least one opening of the insert, and wherein the porous layer comprises a structure and arrangement relative to the skin tissue sample so as to promote mass transport of reagents and/or fluid into the skin tissue sample for supporting viability of the skin tissue sample for at least one week.

16. The ex vivo skin tissue system of claim 15, wherein the adipose layer comprises human tissue from a same source as the skin tissue sample.

17. The ex vivo skin tissue system of claim 15, wherein the adipose layer comprises one or more of human adipose-derived stem cells, adipocytes, or pre-adipocytes.

18. The ex vivo skin tissue system of claim 15, wherein the porous layer comprises a plurality of pores.

19. The ex vivo skin tissue system of claim 15, further comprising at least one of an inlet channel and outlet channel positioned lateral to the porous layer.

20. The ex vivo skin tissue system of claim 15, further comprising a gel layer in contact with the second side, wherein the gel layer is configured to receive one or more of adipose-derived cells, dermal cells, and endothelial cells.

21. The ex vivo skin tissue system of claim 15, further comprising a cell composition seeded within the porous layer between the first side and the second side.

22. The ex vivo skin tissue system of claim 21, wherein the seeded cell composition comprises one or more of endothelial cells, primary cells, iPSCs, keratinocytes, fibroblasts, melanocytes, resident immune cells, circulating immune cells, stem cells, adipocytes, microbes, or a combination thereof.

23. The ex vivo skin tissue system of claim 15, wherein the skin tissue sample comprises at least one of an epidermis layer, a dermis layer, and a hypodermis layer.

24. The ex vivo skin tissue system of claim 15, wherein the porous layer comprises a porosity gradient, and wherein the porosity gradient comprises a lower density of pores in a center of the porous layer relative to an outer boundary of the porous layer.

25. The ex vivo skin tissue system of claim 15, wherein an interior surface of the wall perpendicular to the first side of the porous layer comprises a plurality of pores.

26. The ex vivo skin tissue system of claim 25, wherein the plurality of pores penetrate a desired thickness of the wall.

27. The ex vivo skin tissue system of claim 15, wherein the skin tissue sample is cultured, thereby forming an ex vivo skin model.
